(19) United States
(12) Patent Application Publication
(10) Pub. No.: US 2024/0336680 A1
(43) Pub. Date: Oct. 10, 2024

(54) NOVEL CLDN 18.2-SPECIFIC MONOCLONAL ANTIBODIES AND METHODS OF USE THEREOF

(71) Applicant: ACCURUS BIOSCIENCES, INC., Richmond, CA (US)

(72) Inventors: Haishan Lin, Richmond, CA (US); Richard Zhang, Richmond, CA (US)

(73) Assignee: ACCURUS BIOSCIENCES, INC., Richmond, CA (US)

(21) Appl. No.: 18/753,205

(22) Filed: Jun. 25, 2024

Related U.S. Application Data
(62) Division of application No. 17/262,351, filed on Jan. 22, 2021, now Pat. No. 12,054,543.
(60) Provisional application No. 62/703,036, filed on Jul. 25, 2018.

(51) Int. Cl. C07K 16/28; A61K 47/68; C07K 14/705; C07K 14/725; G01N 33/68

(52) U.S. Cl. CPC C07K 16/28; A61K 47/6849; C07K 14/7051; C07K 14/70517; C07K 14/70578; G01N 33/6854; C07K 2317/24; C07K 2317/31; C07K 2317/33; C07K 2317/565; C07K 2317/77; C07K 2317/92; C07K 2319/02; C07K 2319/03; C07K 2319/30; C07K 2319/33

(57) ABSTRACT

Provided herein are novel anti-CLDN 18.2 antibodies and chimeric antigen receptors (CAR), cells or compositions comprising the same, vector or plasmid encoding anti-CLDN 18.2 CAR, anti-CLDN18.2 antibody-drug conjugates (ADCs), bispecific antibodies containing anti-CLDN 18.2 antibody, and methods for producing the same, or using the same for detecting or treating ovarian cancer or prostate cancer. Also provided herein are anti-CLDN 18.2 antibody, compositions comprising the same, nucleic acid sequence encoding the same, and a kit for detecting CLDN 18.2.

### CROSS-REFERENCE TO RELATED APPLICATION

This application is a divisional of U.S. patent application Ser. No. 17/262,351, filed Jan. 22, 2021, which is a national stage entry under U.S.C. § 371 of International Application No. PCT/US2019/043316, filed Jul. 24, 2019, which claims priority under 35 U.S.C. § 119 (e) of U.S. Provisional Application No. 62/703,036, filed Jul. 25, 2018, the content of each of which is hereby incorporated by reference into the present disclosure in their entireties.

### SEQUENCE LISTING

The instant application contains a Sequence Listing which has been submitted electronically in XML format and is hereby incorporated by reference in its entirety. Said XML copy, created on May 17, 2024, is named 119163-0124_SL.xml and is 138,191 bytes in size.

### TECHNICAL FIELD

This disclosure relates to novel CLDN 18.2 antibodies and chimeric antigen receptors (CAR) derived therefrom, cells or compositions comprising the same, and methods for using the same for therapy including the treatment of solid tumors.

### BACKGROUND

Isoform 2 of claudin-18 (CLDN 18.2), a tight junction molecule, is known to be a selective lineage marker. It has been observed to be expressed in a significant proportion of primary gastric cancers and metastases. Extopic activation has also been seen in pancreatic, esophageal, ovarian, and lung tumors. While it has a highly restricted expression in normal tissues, CLDN 18.2 is ectopically activated in a diversity of cancers. See Sahin et al., Claudin-18 splice variant 2 is a pan-cancer target suitable for therapeutic antibody development, Clin. Cancer Res. 2008; 14) 23 (7624-34. Accordingly, antibodies against this isoform are useful in the treatment of treatment of gastrointestinal (GI) tumors, such as gastric, pancreatic, esophageal, ovarian, and lung tumors (e.g., IMAB362 (claudiximab, developed by Ganymed Pharmaceuticals AG)).

### SUMMARY OF THE DISCLOSURE

This disclosure provides a novel antibody to CLDN 18.2 and compositions and methods of use thereof. More specifically, this disclosure provides new anti-CLDN 18.2 antibodies for the treatment of tumors expressing CLDN 18.2, such as for example, GI tumors that include, but are not limited to, gastric, pancreatic, esophageal, biliary tract, colorectal, colon, rectal, ovarian, and lung carcinomas. The target, CLDN 18.2, is highly expressed on the majority of these tumors but has restricted off-target positivity and therefore a desirable safety profile. Thus, in one aspect, the compositions are particularly useful in the treatment of tumors or cancerous cell that express or overexpress CLDN 18.2.

In one aspect, the present disclosure provides isolated antibodies, the antibodies comprising a heavy chain (HC) immunoglobulin variable domain sequence and a light chain (LC) immunoglobulin variable domain sequence, wherein the antibodies bind to an epitope of CLDN 18.2. In one aspect, the antibodies bind to an epitope of CLDN 18.2, but not CLDN 18.1. In one aspect, the antibody is a humanized antibody comprising one or more, two or more, three or more, four or more, five or more, six or more, seven or more or all eight of the CDRs identified in Table 3, with the antibodies disclosed in Table 4. In another aspect, the antibodies bind with high specificity, that is they bind to CLDN 18.2 with an affinity of more than 80%, or alternatively more than 85%, or alternatively more than 90%, or alternatively more than 95%, binding for CLDN18.2-expressing cells versus less than 20%, or alternatively less than 15%, or alternatively less than 10%, or alternatively less than 5% binding to CLDN 18.1. Examples of such are shown in FIGS. 1a, 1b and 9. There, the antibodies are more specific for the CLDN18.2 expressing tumor cells and would have lower toxicity since they do not bind to the CLDN18.1-expressing normal cells.

In some embodiments, the antibody is a bispecific antibody has a binding specificity to a second target protein. The second target protein, in some embodiments, is selected from the group consisting of IL-1, CD3, CD16, CD19, CD28, CD47, CD64, PD-1, PD-L1, CTLA-4, LAG-3, CD28, CD122, 4-1BB, TIM3, OX-40, OX40L, CD40, CD40L, LIGHT, ICOS, ICOSL, GITR, GITRL, TIGIT, CD27, VISTA, B7H3, B7H4, HEVM, BTLA and KIR. In some embodiments, the antibody is comprised in an antibody drug conjugates (ADC) formed between the antibody and another agent, such as toxin or drug. In some embodiments, the antibodies may be conjugated to therapeutic agents, prodrugs, peptides, proteins, enzymes, viruses, lipids, biological response modifiers, pharmaceutical agents, and/or polyethylene glycol (PEG).

In a further aspect, this disclosure provides isolated anti-CLDN 18.2 antibodies or fragments thereof as disclosed herein and a detectable or purification label, alone or in combination with an CLDN 18.2 antigen or fragment (e.g., VH1, VH2, VH3, VL1, VL2, VL3, heavy and light chain CDR 1, CDR 2, CDR3) thereof, that in one aspect are bound to each other to form a antigen/antibody complex. Further provided herein is an ex vivo cell comprising this antigen/antibody complex.

Aspects of the disclosure relate to a chimeric antigen receptor (CAR) comprising: (a) an antigen binding domain of a CLDN 18.2 antibody (e.g., VH1, VH2, VH3, VL1, VL2, VL3, heavy and light chain CDR 1, CDR 2, CDR3); (b) a hinge domain; (c) a transmembrane domain; and (d) an intracellular domain. Further aspects of the disclosure relate to a chimeric antigen receptor (CAR) comprising: (a) an antigen binding domain of a CLDN 18.2 antibody (e.g., VH1, VH2, VH3, VL1, VL2, VL3, heavy and light chain CDR 1, CDR 2, CDR3); (b) a hinge domain; (c) a CD28 transmembrane domain; (d) one or more costimulatory regions selected from a CD28 costimulatory signaling region, a 4-1BB costimulatory signaling region, an ICOS costimulatory signaling region, and an OX40 costimulatory region; and (e) a CD3 zeta signaling domain and alternatives thereof.

In a further aspect, the present disclosure provides a chimeric antigen receptor (CAR) comprising: (a) an antigen binding domain of an anti-CLDN 18.2 antibody (e.g., VH1, VH2, VH3, VL1, VL2, VL3, heavy and light chain CDR 1, CDR 2, CDR3), (b) a CD8α hinge domain; (c) a CD8α transmembrane domain; (d) a CD28 and/or a 4-1BB costimulatory signaling region; and (e) a CD3 zeta signaling domain and alternatives thereof.

In another aspect, the present disclosure provides an isolated nucleic acid sequence encoding the anti-CLDN 18.2 antibody, chimeric antibody, humanized antibody, bispecific antibody, or the anti-CLDN 18.2 CAR.

In another aspect, the present disclosure provides a vector comprising the isolated nucleic acid sequence encoding the anti-CLDN 18.2 antibody, chimeric antibody, bispecific antibody, humanized antibody, or the anti-CLDN 18.2 CAR.

In another aspect, the present disclosure provides a vector comprising the isolated nucleic acid sequence encoding the anti-CLDN 18.2 antibody, chimeric antibody, bispecific antibody, humanized antibody, or the anti-CLDN 18.2 CAR.

In another aspect, the present disclosure provides a composition comprising a carrier and one or more of: the anti-CLDN 18.2 antibody; and/or the bispecific antibody; and/or the chimeric antibody; and/or the humanized antibody; and/or the anti-CLDN 18.2 CAR; and/or the isolated nucleic acid encoding the anti-CLDN 18.2 antibody bispecific antibody, chimeric antibody, humanized antibody, or the anti-CLDN 18.2 CAR; and/or the vector comprising the isolated nucleic acid sequence encoding the anti-CLDN 18.2 antibody, bispecific antibody, chimeric antibody, humanized antibody, or the anti-CLDN 18.2 CAR; and/or an isolated cell comprising the anti-CLDN 18.2 CAR.

In one aspect, the disclosure provides a composition comprising, or alternatively consisting essentially of, or yet further consisting of a carrier and one or more of: an antibody or fragment (e.g., VH1, VH2, VH3, VL1, VL2, VL3, heavy and light chain CDR 1, CDR 2, CDR3) thereof, a nucleic acid encoding the antibody or fragment (e.g., VH1, VH2, VH3, VL1, VL2, VL3, heavy and light chain CDR 1, CDR 2, CDR3) thereof, a vector comprising the nucleic acid encoding the antibody or fragment (e.g., VH1, VH2, VH3, VL1, VL2, VL3, heavy and light chain CDR 1, CDR 2, CDR3) thereof, an isolated cell comprising an anti-CLDN 18.2 CAR and/or a polynucleotide encoding the antibody or fragment (e.g., VH1, VH2, VH3, VL1, VL2, VL3, heavy and light chain CDR 1, CDR 2, CDR3) thereof; and/or the isolated nucleic acid encoding the CAR; and/or the vector comprising the nucleic acid encoding the CAR; and/or the isolated cell expressing an anti-CLDN 18.2 CAR; and/or the anti-CLDN 18.2 antibody; and/or bispecific antibody; and/or humanized antibody.

Compositions are also provided. In one embodiment, the composition comprises an antibody or fragment (e.g., VH1, VH2, VH3, VL1, VL2, VL3, heavy and light chain CDR 1, CDR 2, CDR3) thereof, a fusion protein, or a CAR and/or car expressing-cell of the present disclosure and a pharmaceutically acceptable carrier. Also provided is an isolated cell comprising one or more polynucleotide encoding the antibody or fragment (e.g., VH1, VH2, VH3, VL1, VL2, VL3, heavy and light chain CDR 1, CDR 2, CDR3) thereof or the fusion protein or bispecific antibody of the present disclosure.

The present disclosure further provides a method of treating tumor or growth expressing CLND 18.2 antigen, such as for example cancer in a patient in need thereof, comprising administering to the patient a antibody or fragment (e.g., VH1, VH2, VH3, VL1, VL2, VL3, heavy and light chain CDR 1, CDR 2, CDR3) thereof, a fusion protein, or a CAR and/or car expressing-cell of the present disclosure. Non-limiting examples of cancers include GI cancers, bladder cancer, liver cancer, colon cancer, rectal cancer, colorectal cancer, endometrial cancer, biliary cancer, leukemia, lymphoma, pancreatic cancer, small cell lung cancer, non-small cell lung cancer, breast cancer, urethral cancer, head and neck cancer, gastrointestinal cancer, stomach cancer, oesophageal cancer, ovarian cancer, renal cancer, melanoma, prostate cancer and thyroid cancer.

### BRIEF DESCRIPTION OF THE DRAWINGS

FIG. 1A-1B show the binding specificity of anti-CLDN18.2 hybridoma antibodies. CHO stable cell lines expressing either human CLDN18.1 or human CLDN18.2 were used in the FACS analysis. All anti-CLDN18.2 hybridoma antibodies were used at 10 μg/ml.

FIG. 2 shows that anti-CLDN18.2 hybridoma antibody clone T29S9 binds to both CLDN18.1 and CLDN18.2 in the FACS analysis. T29S9 was used at 10 μg/ml in the analysis.

FIG. 3 shows binding of anti-CLDN18.2 hybridoma antibodies with titration to CHO-CLDN18.2 cells.

FIG. 4 shows the CLDN18.2 hybridoma antibody binding to a plate coated with CLDN18.2 EC1 Protein. Human CLDN18.2EC1-huFc protein was coated onto ELISA wells at 1 μg/ml.

FIG. 5 shows the binding of Anti-CLDN18.2 hybridoma antibodies to KATO III Cells. Anti-CLDN18.2 hybridoma antibodies were used at 20 μg/ml in the FACS analysis.

FIG. 6 shows binding of anti-CLDN18.2 chimeric antibodies with titration to CHO-CLDN18.2 cells in the FACS analysis.

FIG. 7 shows binding of anti-CLDN18.2 chimeric antibodies to KATO III cells. Anti-CLDN18.2 chimeric antibodies were used at 20 μg/ml in the FACS analysis.

FIG. 8A-8E show anti-human CLDN18.2 antibody-induced receptor internalization in CHO-CLDN18.2 cells.

FIG. 9 shows humanized anti-CLDN18.2 antibodies, HuT252S4, bind to cell surface human CLDN18.2 on CHO cells stably expressing CLDN18.2 at similar or higher affinity as the chimeric T252S4 antibody.

### DETAILED DESCRIPTION

It is to be understood that the present disclosure is not limited to particular aspects described, as such may, of course, vary. It is also to be understood that the terminology used herein is for the purpose of describing particular aspects only, and is not intended to be limiting, since the scope of the present disclosure will be limited only by the appended claims.

Unless defined otherwise, all technical and scientific terms used herein have the same meanings as commonly understood by one of ordinary skill in the art to which this technology belongs. Although any methods and materials similar or equivalent to those described herein can be used in the practice or testing of the present technology, the preferred methods, devices and materials are now described. All technical and patent publications cited herein are incorporated herein by reference in their entirety. Nothing herein is to be construed as an admission that the present technology is not entitled to antedate such disclosure by virtue of prior invention.

The practice of the present technology will employ, unless otherwise indicated, conventional techniques of tissue culture, immunology, molecular biology, microbiology, cell biology, and recombinant DNA, which are within the skill of the art. See, e.g., Green and Sambrook eds. (2012) Molecular Cloning: A Laboratory Manual, 4^{th} edition; the series Ausubel et al. eds. (2015) Current Protocols in Molecular Biology; the series Methods in Enzymology (Academic Press, Inc., N.Y.); MacPherson et al. (2015) PCR 1: A Practical Approach (IRL Press at Oxford University Press); MacPherson et al. (1995) PCR 2: A Practical Approach; McPherson et al. (2006) PCR: The Basics (Garland Science); Harlow and Lane eds. (1999) Antibodies, A Laboratory Manual; Greenfield ed. (2014) Antibodies, A Laboratory Manual; Freshney (2010) Culture of Animal Cells: A Manual of Basic Technique, 6^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Herdewijn ed. (2005) Oligonucleotide Synthesis: Methods and Applications; Hames and Higgins eds. (1984) Transcription and Translation; Buzdin and Lukyanov ed. (2007) Nucleic Acids Hybridization: Modern Applications; Immobilized Cells and Enzymes (IRL Press (1986)); Grandi ed. (2007) In Vitro Transcription and Translation Protocols, 2^{nd} edition; Guisan ed. (2006) Immobilization of Enzymes and Cells; Perbal (1988) A Practical Guide to Molecular Cloning, 2^{nd} edition; Miller and Calos eds, (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Lundblad and Macdonald eds. (2010) Handbook of Biochemistry and Molecular Biology, 4^{th} edition; and Herzenberg et al. eds (1996) Weir's Handbook of Experimental Immunology, 5^{th} edition; and the more recent editions each thereof available at the time of filing.

All numerical designations, fus e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about.” It also is to be understood, although not always explicitly stated, that the reagents described herein are merely exemplary and that equivalents of such are known in the art.

It is to be inferred without explicit recitation and unless otherwise intended, that when the present technology relates to a polypeptide, protein, polynucleotide or antibody, an equivalent or a biologically equivalent of such is intended within the scope of the present technology.

### Definitions

As used in the specification and claims, the singular form “a”, “an”, and “the” include plural references unless the context clearly dictates otherwise. For example, the term “a cell” includes a plurality of cells, including mixtures thereof.

As used herein, the term “animal” refers to living multi-cellular vertebrate organisms, a category that includes, for example, mammals and birds. The term “mammal” includes both human and non-human mammals.

The terms “subject,” “host,” “individual,” and “patient” are as used interchangeably herein to refer to human and veterinary subjects, for example, humans, animals, non-human primates, dogs, cats, sheep, mice, horses, and cows. In some embodiments, the subject is a human.

As used herein, the term “antibody” collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits and mice, as well as non-mammalian species, such as shark immunoglobulins. Unless specifically noted otherwise, the term “antibody” includes intact immunoglobulins and “antibody fragments” or “antigen binding fragments” that specifically bind to a molecule of interest (or a group of highly similar molecules of interest) to the substantial exclusion of binding to other molecules (for example, antibodies and antibody fragments that have a binding constant for the molecule of interest that is at least 10³ M⁻¹ greater, at least 10⁴ M⁻¹ greater or at least 10⁵ M⁻¹ greater than a binding constant for other molecules in a biological sample). The term “antibody” also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Owen et al., Kuby Immunology, 7^{th} Ed., W.H. Freeman & Co., 2013; Murphy, Janeway's Immunobiology, 8^{th} Ed., Garland Science, 2014; Male et al., Immunology (Roitt), 8^{th} Ed., Saunders, 2012; Parham, The Immune System, 4^{th} Ed., Garland Science, 2014.

In terms of antibody structure, an immunoglobulin has heavy (H) chains and light (L) chains interconnected by disulfide bonds. There are two types of light chain, lambda (2) and kappa (κ). There are five main heavy chain classes (or isotypes) which determine the functional activity of an antibody molecule: IgM, IgD, IgG, IgA and IgE. Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, largely adopts a β-sheet conformation and the CDRs form loops which connect, and in some cases form part of, the β-sheet structure. Thus, framework regions act to form a scaffold that provides for positioning the CDRs in correct orientation by inter-chain, non-covalent interactions.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. An antibody that binds CLDN 18.2 will have a specific V_{H} region and the V_{L} region sequence, and thus specific CDR sequences. Antibodies with different specificities (i.e. different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

As used herein, the term “antigen” refers to a compound, composition, or substance that may be specifically bound by the products of specific humoral or cellular immunity, such as an antibody molecule or T-cell receptor. Antigens can be any type of molecule including, for example, haptens, simple intermediary metabolites, sugars (e.g., oligosaccharides), lipids, and hormones as well as macromolecules such as complex carbohydrates (e.g., polysaccharides), phospholipids, and proteins. Common categories of antigens include, but are not limited to, viral antigens, bacterial antigens, fungal antigens, protozoa and other parasitic antigens, tumor antigens, antigens involved in autoimmune disease, allergy and graft rejection, toxins, and other miscellaneous antigens.

As used herein, the term “antigen binding domain” refers to any protein or polypeptide domain that can specifically bind to an antigen target.

As used herein, the term “autologous,” in reference to cells refers to cells that are isolated and infused back into the same subject (recipient or host). “Allogeneic” refers to non-autologous cells.

As used herein, the term “B cell,” refers to a type of lymphocyte in the humoral immunity of the adaptive immune system. B cells principally function to make antibodies, serve as antigen presenting cells, release cytokines, and develop memory B cells after activation by antigen interaction. B cells are distinguished from other lymphocytes, such as T cells, by the presence of a B-cell receptor on the cell surface. B cells may either be isolated or obtained from a commercially available source. Non-limiting examples of commercially available B cell lines include lines AHH-1 (ATCC® CRL-8146™ (BC-1 (ATCC® CRL-2230™ (BC-2 (ATCC® CRL-2231™ (BC-3 (ATCC® CRL-2277™ (CA46 (ATCC® CRL-1648™ (DG-75 [D.G.-75] (ATCC® CRL-2625™ (DS-1 (ATCC® CRL-11102™ (EB-3 [EB3] (ATCC® CCL-85™ (Z-138 (ATCC #CRL-3001), DB (ATCC CRL-2289), Toledo (ATCC CRL-2631), Pfiffer (ATCC CRL-2632), SR (ATCC CRL-2262), JM-1 (ATCC CRL-10421), NFS-5 C-1 (ATCC CRL-1693); NFS-70 C10 (ATCC CRL-1694), NFS-25 C-3 (ATCC CRL-1695), AND SUP-B15 (ATCC CRL-1929). Further examples include but are not limited to cell lines derived from anaplastic and large cell lymphomas, e.g., DEL, DL-40, FE-PD, JB6, Karpas 299, Ki-JK, Mac-2A Ply1, SR-786, SU-DHL-1, -2, -4, -5, -6, -7, -8, -9, -10, and -16, DOHH-2, NU-DHL-1, U-937, Granda 519, USC-DHL-1, RL; Hodgkin's lymphomas, e.g., DEV, HD-70, HDLM-2, HD-MyZ, HKB-1, KM-H2, L 428, L 540, L1236, SBH-1, SUP-HD1, SU/RH-HD-1. Non-limiting exemplary sources for such commercially available cell lines include the American Type Culture Collection, or ATCC, (atcc.org/) and the German Collection of Microorganisms and Cell Cultures (dsmz.de/).

As used herein, a “cancer” is a disease state characterized by the presence in a subject of cells demonstrating abnormal uncontrolled replication and in some aspects, the term may be used interchangeably with the term “tumor.” The term “cancer or tumor antigen” referees to an antigen known to be associated and expressed on the surface with a cancer cell or tumor cell or tissue, and the term “cancer or tumor targeting antibody” refers to an antibody that targets such an antigen.

The term “chimeric antigen receptor” (CAR), as used herein, refers to a fused protein comprising an extracellular domain capable of binding to an antigen, a transmembrane domain derived from a polypeptide different from a polypeptide from which the extracellular domain is derived, and at least one intracellular domain. The “chimeric antigen receptor (CAR)” is sometimes called a “chimeric receptor”, a “T-body”, or a “chimeric immune receptor (CIR).” The “extracellular domain capable of binding to an antigen” means any oligopeptide or polypeptide that can bind to a certain antigen. The “intracellular domain” or “intracellular signaling domain” means any oligopeptide or polypeptide known to function as a domain that transmits a signal to cause activation or inhibition of a biological process in a cell. In certain embodiments, the intracellular domain may comprise, alternatively consist essentially of, or yet further comprise one or more costimulatory signaling domains in addition to the primary signaling domain. The “transmembrane domain” means any oligopeptide or polypeptide known to span the cell membrane and that can function to link the extracellular and signaling domains. A chimeric antigen receptor may optionally comprise a “hinge domain” which serves as a linker between the extracellular and transmembrane domains. Non limiting examples of polynucleotides encoding such domains are provided herein, e.g.:

Hinge domain: IgG1 heavy chain hinge sequence:

(SEQ ID NO: 75(
CTCGAGCCCAAATCTTGTGACAAAACTCACACATGCCCACCGTGCCCG

Transmembrane domain: CD28 transmembrane region:

(SEQ ID NO: 76(
TTTTGGGTGCTGGTGGTGGTTGGTGGAGTCCTGGCTTGCTATAGCTTG
CTAGTAACAGTGGCCTTTATTATTTTCTGGGTG

Intracellular domain: 4-1BB co-stimulatory signaling region:

(SEQ ID NO: 77(
AAACGGGGCAGAAAGAAACTCCTGTATATATTCAAACAACCATTTATG
AGACCAGTACAAACTACTCAAGAGGAAGATGGCTGTAGCTGCCGATTT
CCAGAAGAAGAAGAAGGAGGATGTGAACTG

Intracellular domain: CD28 co-stimulatory signaling region:

(SEQ ID NO: 78(
AGGAGTAAGAGGAGCAGGCTCCTGCACAGTGACTACATGAACATGACT
CCCCGCCGCCCCGGGCCCACCCGCAAGCATTACCAGCCCTATGCCCCA
CCACGCGACTTCGCAGCCTATCGCTCC

Intracellular domain: CD3 zeta signaling region:

(SEQ ID NO: 79(
AGAGTGAAGTTCAGCAGGAGCGCAGACGCCCCCGCGTACCAGCAGGGCCA
GAACCAGCTCTATAACGAGCTCAATCTAGGACGAAGAGAGGAGTACGATG
TTTTGGACAAGAGACGTGGCCGGGACCCTGAGATGGGGGGAAAGCCGAGA
AGGAAGAACCCTCAGGAAGGCCTGTACAATGAACTGCAGAAAGATAAGAT
GGCGGAGGCCTACAGTGAGATTGGGATGAAAGGCGAGCGCCGGAGGGGCA
AGGGGCACGATGGCCTTTACCAGGGTCTCAGTACAGCCACCAAGGACACC
TACGACGCCCTTCACATGCAGGCCCTGCCCCCTCGCTAA,

or equivalents of each thereof.

Further embodiments of each exemplary domain component include other proteins that have analogous biological function that share at least 70%, or alternatively at least 80% amino acid sequence identity, preferably 90% sequence identity, more preferably at least 95% sequence identity with the proteins encoded by the above disclosed nucleic acid sequences. Further, non limiting examples of such domains are provided herein.

As used herein, the term “CD8α hinge domain” refers to a specific protein fragment associated with this name and any other molecules that have analogous biological function that share at least 70%, or alternatively at least 80% amino acid sequence identity, preferably 90% sequence identity, more preferably at least 95% sequence identity with the CD8α hinge domain sequence as shown herein. The example sequences of CD8α hinge domain for human, mouse, and other species are provided in Pinto, R. D. et al. (2006) Vet. Immunol. Immunopathol. 110:169-177. The sequences associated with the CD8α hinge domain are provided in Pinto, R. D. et al. (2006) Vet. Immunol. Immunopathol. 110:169-177.

### Non-Limiting Examples of Such Include:

### Human CD8 Alpha Hinge Domain:

(SEQ ID NO: 80(
PAKPTTTPAPRPPTPAPTIASQPLSLRPEACRPAAGGAVHTRGLDFACDI
Y

### Mouse CD8 Alpha Hinge Domain:

(SEQ ID NO: 81(
KVNSTTTKPVLRTPSPVHPTGTSQPQRPEDCRPRGSVKGTGLDFACDIY

### Cat CD8 Alpha Hinge Domain:

(SEQ ID NO: 82(
PVKPTTTPAPRPPTQAPITTSQRVSLRPGTCQPSAGSTVEASGLDLSCDI
Y,

and equivalents of each thereof.

As used herein, the term “CD8α transmembrane domain” refers to a specific protein fragment associated with this name and any other molecules that have analogous biological function that share at least 70%, or alternatively at least 80% amino acid sequence identity, preferably 90% sequence identity, more preferably at least 95% sequence identity with the CD8α transmembrane domain sequence as shown herein. The fragment sequences associated with the amino acid positions 183 to 203 of the human T-cell surface glycoprotein CD8 alpha chain (GenBank Accession No: NP_001759.3), or the amino acid positions 197 to 217 of the mouse T-cell surface glycoprotein CD8 alpha chain (GenBank Accession No: NP_001074579.1), and the amino acid positions 190 to 210 of the rat T-cell surface glycoprotein CD8 alpha chain (GenBank Accession No: NP_113726.1) provide additional example sequences of the CD8α transmembrane domain. The sequences associated with each of the listed accession numbers are provided as follows:

Human CD8 alpha transmembrane domain:
(SEQ ID NO: 83(
IYIWAPLAGTCGVLLLSLVIT
Mouse CD8 alpha transmembrane domain:
(SEQ ID NO: 84(
IWAPLAGICVALLLSLIITLI
Rat CD8 alpha transmembrane domain:
(SEQ ID NO: 85(
IWAPLAGICAVLLLSLVITLI,
and equivalents of each thereof.

As used herein, the term “CD28 transmembrane domain” refers to a specific protein fragment associated with this name and any other molecules that have analogous biological function that share at least 70%, or alternatively at least 80% amino acid sequence identity, at least 90% sequence identity, or alternatively at least 95% sequence identity with the CD28 transmembrane domain sequence as shown herein. The fragment sequences associated with the GenBank Accession Nos: XM_006712862.2 and XM_009444056.1 provide additional, non-limiting, example sequences of the CD28 transmembrane domain. The sequences associated with each of the listed accession numbers are provided herein.

As used herein, the term “4-1BB costimulatory signaling region” refers to a specific protein fragment associated with this name and any other molecules that have analogous biological function that share at least 70%, or alternatively at least 80% amino acid sequence identity, preferably 90% sequence identity, more preferably at least 95% sequence identity with the 4-1BB costimulatory signaling region sequence as shown herein. Non-limiting example sequences of the 4-1BB costimulatory signaling region are provided in U.S. Publication 20130266551A1 (filed as U.S. application Ser. No. 13/826,258), such as the exemplary sequence provided below:

### 4-1BB Costimulatory Signaling Region:

(SEQ ID NO: 86(
KRGRKKLLYIFKQPFMRPVQTTQEEDGCSCRFPEEEEGGCEL,
and an equivalent thereof.

As used herein, the term “CD28 costimulatory signaling region” refers to a specific protein fragment associated with this name and any other molecules that have analogous biological function that share at least 70%, or alternatively at least 80% amino acid sequence identity, preferably 90% sequence identity, more preferably at least 95% sequence identity with the CD28 costimulatory signaling region sequence shown herein. The example sequences CD28 costimulatory signaling domain are provided in U.S. Pat. No. 5,686,281; Geiger, T. L. et al., Blood 98:2364-2371 (2001); Hombach, A. et al., J Immunol 167:6123-6131 (2001); Maher, J. et al. Nat Biotechnol 20:70-75 (2002); Haynes, N. M. et al., J Immunol 169:5780-5786 (2002); Haynes, N. M. et al., Blood 100:3155-3163 (2002). Non-limiting examples include residues 114-220 of the below CD28 Sequence:

(SEQ ID NO: 87(
MLRLLLALNL FPSIQVTGNK ILVKQSPMLV AYDNAVNLSC
KYSYNLFSRE FRASLHKGLDSAVEVCVVYG NYSQQLQVYS
KTGFNCDGKL GNESVTFYLQ NLYVNQTDIY
FCKIEVMYPPPYLDNEKSNG TIIHVKGKHL CPSPLFPGPS
KPFWVLVVVG GVLACYSLLVTVAFIIFWVR SKRSRLLHSD
YMNMTPRRPG PTRKHYQPYA PPRDFAAYRS,
and equivalents thereof.

As used herein, the term “ICOS costimulatory signaling region” refers to a specific protein fragment associated with this name and any other molecules that have analogous biological function that share at least 70%, or alternatively at least 80% amino acid sequence identity, preferably 90% sequence identity, more preferably at least 95% sequence identity with the ICOS costimulatory signaling region sequence as shown herein. Non-limiting example sequences of the ICOS costimulatory signaling region are provided in U.S. Publication 2015/0017141A1 the exemplary polynucleotide encoding this sequence provided below.

### ICOS Costimulatory Signaling Region Encoded by Polynucleotide:

(SEQ ID NO: 88(,
ACAAAAAAGA AGTATTCATC CAGTGTGCAC GACCCTAACG
GTGAATACAT GTTCATGAGA GCAGTGAACA CAGCCAAAAA
ATCCAGACTC ACAGATGTGA CCCTA
and equivalents thereof.

As used herein, the term “OX40 costimulatory signaling region” refers to a specific protein fragment associated with this name and any other molecules that have analogous biological function that share at least 70%, or alternatively at least 80% amino acid sequence identity, or alternatively 90% sequence identity, or alternatively at least 95% sequence identity with the OX40 costimulatory signaling region sequence as shown herein. Non-limiting example sequences of the OX40 costimulatory signaling region are disclosed in U.S. Publication 2012/20148552A1, and include an exemplary sequence encoding such is provided below.

### OX40 Costimulatory Signaling Region Encoded by Polynucleotide:

(SEQ ID NO: 89(
AGGGACCAG AGGCTGCCCC CCGATGCCCA CAAGCCCCCT
GGGGGAGGCA GTTTCCGGAC CCCCATCCAA GAGGAGCAGG
CCGACGCCCA CTCCACCCTG GCCAAGATC,
and equivalents thereof.

As used herein, the term “CD28 costimulatory signaling region” refers to a specific protein fragment associated with this name and any other molecules that have analogous biological function that share at least 70%, or alternatively at least 80% amino acid sequence identity, or alternatively 90% sequence identity, or alternatively at least 95% sequence identity with the CD28 costimulatory signaling region sequence shown herein. The example sequences CD28 costimulatory signaling domain are provided in U.S. Pat. No. 5,686,281; Geiger, T. L. et al. (2001) Blood 98:2364-2371; Hombach, A. et al. (2001) J Immunol 167:6123-6131; Maher, J. et al. (2002) Nat Biotechnol 20:70-75; Haynes, N. M. et al. (2002) J Immunol 169:5780-5786 (2002); Haynes, N. M. et al. (2002) Blood 100:3155-3163. Non-limiting examples include residues 114-220 of the below and the sequence encoded:

CD28 Sequence:
(SEQ ID NO: 87(
MLRLLLALNL FPSIQVTGNK ILVKQSPMLV AYDNAVNLSC
KYSYNLFSRE FRASLHKGLDSAVEVCVVYG NYSQQLQVYS
KTGFNCDGKL GNESVTFYLQ NLYVNQTDIY
FCKIEVMYPPPYLDNEKSNG TIIHVKGKHL CPSPLFPGPS
KPFWVLVVVG GVLACYSLLVTVAFIIFWVR SKRSRLLHSD
YMNMTPRRPG PTRKHYQPYA PPRDFAAYRS,
and equivalents thereof.

As used herein, the term “CD3 zeta signaling domain” refers to a specific protein fragment associated with this name and any other molecules that have analogous biological function that share at least 70%, or alternatively at least 80% amino acid sequence identity, preferably 90% sequence identity, more preferably at least 95% sequence identity with the CD3 zeta signaling domain sequence as shown herein. Non-limiting example sequences of the CD3 zeta signaling domain are provided in U.S. application Ser. No. 13/826,258, e.g.:

(SEQ ID NO: 90(
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR,
and equivalents thereof.

A “composition” typically intends a combination of the active agent, e.g., compound or composition, and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri-, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. For example, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions disclosed herein. Aspects defined by each of these transition terms are within the scope of the present disclosure.

The term “consensus sequence” as used herein refers to an amino acid or nucleic acid sequence that is determined by aligning a series of multiple sequences and that defines an idealized sequence that represents the predominant choice of amino acid or base at each corresponding position of the multiple sequences. Depending on the sequences of the series of multiple sequences, the consensus sequence for the series can differ from each of the sequences by zero, one, a few, or more substitutions. Also, depending on the sequences of the series of multiple sequences, more than one consensus sequence may be determined for the series. The generation of consensus sequences has been subjected to intensive mathematical analysis. Various software programs can be used to determine a consensus sequence.

As used herein, the term “CRISPR” refers to a technique of sequence specific genetic manipulation relying on the clustered regularly interspaced short palindromic repeats pathway. CRISPR can be used to perform gene editing and/or gene regulation, as well as to simply target proteins to a specific genomic location. Gene editing refers to a type of genetic engineering in which the nucleotide sequence of a target polynucleotide is changed through introduction of deletions, insertions, or base substitutions to the polynucleotide sequence. In some aspects, CRISPR-mediated gene editing utilizes the pathways of nonhomologous end-joining (NHEJ) or homologous recombination to perform the edits. Gene regulation refers to increasing or decreasing the production of specific gene products such as protein or RNA.

The term “gRNA” or “guide RNA” as used herein refers to the guide RNA sequences used to target specific genes for correction employing the CRISPR technique. Techniques of designing gRNAs and donor therapeutic polynucleotides for target specificity are well known in the art. For example, Doench, J., et al. Nature biotechnology 2014; 32 (12): 1262-7, Mohr, S. et al. (2016) FEBS Journal 283:3232-38, and Graham, D., et al. Genome Biol. 2015; 16:260. gRNA comprises or alternatively consists essentially of, or yet further consists of a fusion polynucleotide comprising CRISPR RNA (crRNA) and trans-activating CRIPSPR RNA (tracrRNA); or a polynucleotide comprising CRISPR RNA (crRNA) and trans-activating CRIPSPR RNA (tracrRNA). In some aspects, a gRNA is synthetic (Kelley, M. et al. (2016) J of Biotechnology 233 (2016) 74-83). As used herein, a biological equivalent of a gRNA includes but is not limited to polynucleotides or targeting molecules that can guide a Cas9 or equivalent thereof to a specific nucleotide sequence such as a specific region of a cell's genome.

“Cytoreductive therapy,” as used herein, includes but is not limited to chemotherapy, cryotherapy, and radiation therapy. Agents that act to reduce cellular proliferation are known in the art and widely used. Chemotherapy drugs that kill cancer cells only when they are dividing are termed cell-cycle specific. These drugs include agents that act in S-phase, including topoisomerase inhibitors and anti-metabolites.

Topoisomerase inhibitors are drugs that interfere with the action of topoisomerase enzymes (topoisomerase I and II). During the process of chemo treatments, topoisomerase enzymes control the manipulation of the structure of DNA necessary for replication, and are thus cell cycle specific. Examples of topoisomerase I inhibitors include the camptothecin analogs listed above, irinotecan and topotecan. Examples of topoisomerase II inhibitors include amsacrine, etoposide, etoposide phosphate, and teniposide.

Antimetabolites are usually analogs of normal metabolic substrates, often interfering with processes involved in chromosomal replication. They attack cells at very specific phases in the cycle. Antimetabolites include folic acid antagonists, e.g., methotrexate; pyrimidine antagonist, e.g., 5-fluorouracil, floxuridine, cytarabine, capecitabine, and gemcitabine; purine antagonist, e.g., 6-mercaptopurine and 6-thioguanine; adenosine deaminase inhibitor, e.g., cladribine, fludarabine, nelarabine and pentostatin; and the like.

Plant alkaloids are derived from certain types of plants. The vinca alkaloids are made from the periwinkle plant (Catharanthus rosea). The taxanes are made from the bark of the Pacific Yew tree (taxus). The vinca alkaloids and taxanes are also known as antimicrotubule agents. The podophyllotoxins are derived from the May apple plant. Camptothecan analogs are derived from the Asian “Happy Tree” (Camptotheca acuminata). Podophyllotoxins and camptothecan analogs are also classified as topoisomerase inhibitors. The plant alkaloids are generally cell-cycle specific.

Examples of these agents include vinca alkaloids, e.g., vincristine, vinblastine and vinorelbine; taxanes, e.g., paclitaxel and docetaxel; podophyllotoxins, e.g., etoposide and teniposide; and camptothecan analogs, e.g., irinotecan and topotecan.

Cryotherapy includes, but is not limited to, therapies involving decreasing the temperature, for example, hypothermic therapy.

Radiation therapy includes, but is not limited to, exposure to radiation, e.g., ionizing radiation, UV radiation, as known in the art. Exemplary dosages include, but are not limited to, a dose of ionizing radiation at a range from at least about 2 Gy to not more than about 10 Gy and/or a dose of ultraviolet radiation at a range from at least about 5 J/m² to not more than about 50 J/m², usually about 10 J/m².

As used herein, the term “detectable marker” refers to at least one marker capable of directly or indirectly, producing a detectable signal. A non-exhaustive list of this marker includes enzymes which produce a detectable signal, for example by colorimetry, fluorescence, luminescence, such as horseradish peroxidase, alkaline phosphatase, β-galactosidase, glucose-6-phosphate dehydrogenase, chromophores such as fluorescent, luminescent dyes, groups with electron density detected by electron microscopy or by their electrical property such as conductivity, amperometry, voltammetry, impedance, detectable groups, for example whose molecules are of sufficient size to induce detectable modifications in their physical and/or chemical properties, such detection may be accomplished by optical methods such as diffraction, surface plasmon resonance, surface variation, the contact angle change or physical methods such as atomic force spectroscopy, tunnel effect, or radioactive molecules such as ³²P, ³⁵S or ¹²⁵I.

An “effective amount” or “efficacious amount” refers to the amount of an agent, or combined amounts of two or more agents, that, when administered for the treatment of a mammal or other subject, is sufficient to effect such treatment for the disease. The “effective amount” will vary depending on the agent(s), the disease and its severity and the age, weight, etc., of the subject to be treated.

The term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

As used herein, the term “enhancer”, as used herein, denotes sequence elements that augment, improve or ameliorate transcription of a nucleic acid sequence irrespective of its location and orientation in relation to the nucleic acid sequence to be expressed. An enhancer may enhance transcription from a single promoter or simultaneously from more than one promoter. As long as this functionality of improving transcription is retained or substantially retained (e.g., at least 70%, at least 80%, at least 90% or at least 95% of wild-type activity, that is, activity of a full-length sequence), any truncated, mutated or otherwise modified variants of a wild-type enhancer sequence are also within the above definition.

In one aspect, the term “equivalent” or “biological equivalent” of an antibody means the ability of the antibody to selectively bind its epitope protein or fragment thereof as measured by ELISA or other suitable methods. Biologically equivalent antibodies include, but are not limited to, those antibodies, peptides, antibody fragments, antibody variant, antibody derivative and antibody mimetics that bind to the same epitope as the reference antibody.

It is to be inferred without explicit recitation and unless otherwise intended, that when the present disclosure relates to a polypeptide, protein, polynucleotide or antibody, an equivalent or a biologically equivalent of such is intended within the scope of this disclosure. As used herein, the term “biological equivalent thereof” is intended to be synonymous with “equivalent thereof” when referring to a reference protein, antibody, polypeptide or nucleic acid, intends those having minimal homology while still maintaining desired structure or functionality. Unless specifically recited herein, it is contemplated that any polynucleotide, polypeptide or protein mentioned herein also includes equivalents thereof. For example, an equivalent intends at least about 70% homology or identity, or at least 80% homology or identity and alternatively, or at least about 85%, or alternatively at least about 90%, or alternatively at least about 95%, or alternatively 98% percent homology or identity and exhibits substantially equivalent biological activity to the reference protein, polypeptide or nucleic acid. Alternatively, when referring to polynucleotides, an equivalent thereof is a polynucleotide that hybridizes under stringent conditions to the reference polynucleotide or its complement.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample. In one aspect, the expression level of a gene from one sample may be directly compared to the expression level of that gene from a control or reference sample. In another aspect, the expression level of a gene from one sample may be directly compared to the expression level of that gene from the same sample following administration of a compound.

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as “primary therapy and primary treatment.” See National Cancer Institute website at cancer.gov, last visited on May 1, 2008. Typically, a patient is given a subsequent chemotherapy regimen because the patient did not show a positive clinical or sub-clinical response to the first line therapy or the first line therapy has stopped.

As used herein, “homology” or “identical”, percent “identity” or “similarity”, when used in the context of two or more nucleic acids or polypeptide sequences, refers to two or more sequences or subsequences that are the same or have a specified percentage of nucleotides or amino acid residues that are the same, e.g., at least 60% identity, preferably at least 65%, 70%, 75%, 80%, 85%, 90%, 91%, 92%, 93%, 94%, 95%, 96%, 97%, 98%, 99%, or higher identity over a specified region (e.g., nucleotide sequence encoding an antibody described herein or amino acid sequence of an antibody described herein). Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. The terms “homology” or “identical”, percent “identity” or “similarity” also refer to, or can be applied to, the complement of a test sequence. The terms also include sequences that have deletions and/or additions, as well as those that have substitutions. As described herein, the preferred algorithms can account for gaps and the like. Preferably, identity exists over a region that is at least about 25 amino acids or nucleotides in length, or more preferably over a region that is at least 50-100 amino acids or nucleotides in length. An “unrelated” or “non-homologous” sequence shares less than 40% identity, or alternatively less than 25% identity, with one of the sequences disclosed herein.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

The term “isolated” as used herein refers to molecules or biologicals or cellular materials being substantially free from other materials. In one aspect, the term “isolated” refers to nucleic acid, such as DNA or RNA, or protein or polypeptide (e.g., an antibody or derivative thereof), or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

As used herein, the term “isolated cell” generally refers to a cell that is substantially separated from other cells of a tissue.

“Immune cells” includes, e.g., white blood cells (leukocytes) which are derived from hematopoietic stem cells (HSC) produced in the bone marrow, lymphocytes (T cells, B cells, natural killer (NK) cells) and myeloid-derived cells (neutrophil, eosinophil, basophil, monocyte, macrophage, dendritic cells).

As used herein the term “linker sequence” relates to any amino acid sequence comprising from 1 to 10, or alternatively, 8 amino acids, or alternatively 6 amino acids, or alternatively 5 amino acids that may be repeated from 1 to 10, or alternatively to about 8, or alternatively to about 6, or alternatively about 5, or 4 or alternatively 3, or alternatively 2 times. For example, the linker may comprise up to 15 amino acid residues consisting of a pentapeptide repeated three times. In one aspect, the linker sequence is a (Glycine4Serine)3 (SEQ ID NO: 91 (flexible polypeptide linker comprising three copies of gly-gly-gly-gly-ser (SEQ ID NO: 92).

A “normal cell corresponding to the tumor tissue type” refers to a normal cell from a same tissue type as the tumor tissue. A non-limiting example is a normal lung cell from a patient having lung tumor, or a normal colon cell from a patient having colon tumor.

As used herein, the term “T cell,” refers to a type of lymphocyte that matures in the thymus. T cells play an important role in cell-mediated immunity and are distinguished from other lymphocytes, such as B cells, by the presence of a T-cell receptor on the cell surface. T-cells may either be isolated or obtained from a commercially available source. “T cell” includes all types of immune cells expressing CD3 including T-helper cells (CD4+ cells), cytotoxic T-cells (CD8+ cells), natural killer T-cells, T-regulatory cells (Treg) and gamma-delta T cells. A “cytotoxic cell” includes CD8+ T cells, natural-killer (NK) cells, and neutrophils, which cells are capable of mediating cytotoxicity responses. Non-limiting examples of commercially available T-cell lines include lines BCL2 (AAA) Jurkat (ATCC® CRL-2902™), BCL2 (S70A) Jurkat (ATCC® CRL-2900™), BCL2 (S87A) Jurkat (ATCC® CRL-2901™), BCL2 Jurkat (ATCC® CRL-2899™), Neo Jurkat (ATCC® CRL-2898™), TALL-104 cytotoxic human T cell line (ATCC #CRL-11386). Further examples include but are not limited to mature T-cell lines, e.g., such as Deglis, EBT-8, HPB-MLp-W, HUT 78, HUT 102, Karpas 384, Ki 225, My-La, Se-Ax, SKW-3, SMZ-1 and T34; and immature T-cell lines, e.g., ALL-SIL, Be13, CCRF-CEM, CML-T1, DND-41, DU.528, EU-9, HD-Mar, HPB-ALL, H-SB2, HT-1, JK-T1, Jurkat, Karpas 45, KE-37, KOPT-K1, K-T1, L-KAW, Loucy, MAT, MOLT-1, MOLT 3, MOLT-4, MOLT 13, MOLT-16, MT-1, MT-ALL, P12/Ichikawa, Peer, PER0117, PER-255, PF-382, PFI-285, RPMI-8402, ST-4, SUP-T1 to T14, TALL-1, TALL-101, TALL-103/2, TALL-104, TALL-105, TALL-106, TALL-107, TALL-197, TK-6, TLBR-1, -2, -3, and -4, CCRF-HSB-2 (CCL-120.1), J.RT3-T3.5 (ATCC TIB-153), J45.01 (ATCC CRL-1990), J.CaM1.6 (ATCC CRL-2063), RS4; 11 (ATCC CRL-1873), CCRF-CEM (ATCC CRM-CCL-119); and cutaneous T-cell lymphoma lines, e.g., HuT78 (ATCC CRM-TIB-161), MJ [G11] (ATCC CRL-8294), HuT102 (ATCC TIB-162). Null leukemia cell lines, including but not limited to REH, NALL-1, KM-3, L92-221, are a another commercially available source of immune cells, as are cell lines derived from other leukemias and lymphomas, such as K562 erythroleukemia, THP-1 monocytic leukemia, U937 lymphoma, HEL erythroleukemia, HL60 leukemia, HMC-1 leukemia, KG-1 leukemia, U266 myeloma. Non-limiting exemplary sources for such commercially available cell lines include the American Type Culture Collection, or ATCC, (atcc.org/) and the German Collection of Microorganisms and Cell Cultures (dsmz.de/).

As used herein, the term “NK cell,” also known as natural killer cell, refers to a type of lymphocyte that originates in the bone marrow and play a critical role in the innate immune system. NK cells provide rapid immune responses against viral-infected cells, tumor cells or other stressed cell, even in the absence of antibodies and major histocompatibility complex on the cell surfaces. NK cells may either be isolated or obtained from a commercially available source. Non-limiting examples of commercial NK cell lines include lines NK-92 (ATCC® CRL-2407™), NK-92 MI (ATCC® CRL-2408™). Further examples include but are not limited to NK lines HANK1, KHYG-1, NKL, NK-YS, NOI-90, and YT. Non-limiting exemplary sources for such commercially available cell lines include the American Type Culture Collection, or ATCC, (atcc.org/) and the German Collection of Microorganisms and Cell Cultures (dsmz.de/).

As used herein in reference to a regulatory polynucleotide, the term “operatively linked” refers to an association between the regulatory polynucleotide and the polynucleotide sequence to which it is linked such that, when a specific protein binds to the regulatory polynucleotide, the linked polynucleotide is transcribed.

As used herein, the term “overexpress” with respect to a cell, a tissue, or an organ expresses a protein to an amount that is greater than the amount that is produced in a control cell, a control issue, or an organ. A protein that is overexpressed may be endogenous to the host cell or exogenous to the host cell.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any aspect of this technology that is a polynucleotide encompasses both the double-stranded form and each of two complementary single-stranded forms known or predicted to make up the double-stranded form.

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The term “promoter” as used herein refers to any sequence that regulates the expression of a coding sequence, such as a gene. Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors.

The term “protein”, “peptide” and “polypeptide” are used interchangeably and in their broadest sense to refer to a compound of two or more subunit amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

As used herein, the term “purified” does not require absolute purity; rather, it is intended as a relative term. Thus, for example, a purified nucleic acid, peptide, protein, biological complexes or other active compound is one that is isolated in whole or in part from proteins or other contaminants. Generally, substantially purified peptides, proteins, biological complexes, or other active compounds for use within the disclosure comprise more than 80% of all macromolecular species present in a preparation prior to admixture or formulation of the peptide, protein, biological complex or other active compound with a pharmaceutical carrier, excipient, buffer, absorption enhancing agent, stabilizer, preservative, adjuvant or other co-ingredient in a complete pharmaceutical formulation for therapeutic administration. More typically, the peptide, protein, biological complex or other active compound is purified to represent greater than 90%, often greater than 95% of all macromolecular species present in a purified preparation prior to admixture with other formulation ingredients. In other cases, the purified preparation may be essentially homogeneous, wherein other macromolecular species are not detectable by conventional techniques.

As used herein, the term “purification marker” refers to at least one marker useful for purification or identification. A non-exhaustive list of this marker includes His, lacZ, GST, maltose-binding protein, NusA, BCCP, c-myc, CaM, FLAG, GFP, YFP, cherry, thioredoxin, poly (NANP), V5, Snap, HA, chitin-binding protein, Softag 1, Softag 3, Strep, or S-protein. Suitable direct or indirect fluorescence marker comprise FLAG, GFP, YFP, RFP, dTomato, cherry, Cy3, Cy 5, Cy 5.5, Cy 7, DNP, AMCA, Biotin, Digoxigenin, Tamra, Texas Red, rhodamine, Alexa fluors, FITC, TRITC or any other fluorescent dye or hapten.

As used herein, the term “recombinant protein” refers to a polypeptide which is produced by recombinant DNA techniques, wherein generally, DNA encoding the polypeptide is inserted into a suitable expression vector which is in turn used to transform a host cell to produce the heterologous protein.

As used herein, the term “specific binding” means the contact between an antibody and an antigen with a binding affinity of at least 10⁶ M. In certain aspects, antibodies bind with affinities of at least about 10⁻⁷ M, and preferably 10⁸ M, 10⁹ M, 10¹⁰ M, 10¹¹ M, or 10¹² M.

As used herein, the term “fusion protein” or “fusion polypeptide” intends any drug (small molecule, polypeptide or protein) fused or joined to an antibody or fragment thereof as disclosed herein. Non-limiting examples of such includes, for example, bispecific and antibody-drug conjugates

A “solid tumor” is an abnormal mass of tissue that usually does not contain cysts or liquid areas. Solid tumors can be benign or malignant, metastatic or non-metastatic. Different types of solid tumors are named for the type of cells that form them. Examples of solid tumors include sarcomas, carcinomas, and lymphomas.

As used herein, the term “suicide gene” is a gene capable of inducing cell apoptosis; non-limiting examples include HSV-TK (Herpes simplex virus thymidine kinase), cytosine deaminase, nitroreductase, carboxylesterase, cytochrome P450 or PNP (Purine nucleoside phosphorylase), truncated EGFR, or inducible caspase (“iCasp”). Suicide genes may function along a variety of pathways, and, in some cases, may be inducible by an inducing agent such as a small molecule. For example, the iCasp suicide gene comprises portion of a caspase protein operatively linked to a protein optimized to bind to an inducing agent; introduction of the inducing agent into a cell comprising the suicide gene results in the activation of caspase and the subsequent apoptosis of said cell.

The term “transduce” or “transduction” as it is applied to the production of chimeric antigen receptor cells refers to the process whereby a foreign nucleotide sequence is introduced into a cell. In some embodiments, this transduction is done via a vector.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. Treatments containing the disclosed compositions and methods can be first line, second line, third line, fourth line, fifth line therapy and are intended to be used as a sole therapy or in combination with other appropriate therapies. In one aspect, the term “treatment” excludes prevention.

As used herein, the term “vector” refers to a nucleic acid construct deigned for transfer between different hosts, including but not limited to a plasmid, a virus, a cosmid, a phage, a BAC, a YAC, etc. In some embodiments, plasmid vectors may be prepared from commercially available vectors. In other embodiments, viral vectors may be produced from baculoviruses, retroviruses, adenoviruses, AAVs, etc. according to techniques known in the art. In one embodiment, the viral vector is a lentiviral vector.

As used herein the term “CLDN 18.2” refers to isoform 2 of claudin-18. Sec Sahin U, Koslowski M, Dhaene K, Usener D, Brandenburg G, Seitz G, Huber C, Tureci O. Claudin-18 splice variant 2 is a pan-cancer target suitable for therapeutic antibody development, Clin. Cancer Res. 2008; 14 (23): 7624-34. Non-limiting exemplary sequences of claudin 18 or the underlying gene may be found under Gene Cards ID: GC03P137998, HGNC: 2039, Entrez Gene: 51208, Ensembl: ENSG00000066405, OMIM: 609210, and UniProtKB: P56856, which are incorporated by reference herein.

The sequences associated with each of the above listed GenBank Accession Nos. and references are herein incorporated by reference.

### Modes for Carrying Out the Disclosure

This disclosure provides a novel antibody to CLDN 18.2 and compositions and methods of use thereof. More specifically, this disclosure provides new anti-CLDN 18.2 antibodies for the treatment of tumors expressing CLDN 18.2, such as for example, GI tumors that include, but are not limited to, gastric, pancreatic, esophageal, biliary tract, colorectal, ovarian, and lung carcinomas. The target, CLDN 18.2, is highly expressed on the majority of these tumors but has restricted off-target positivity and therefore a desirable safety profile. Thus, in one aspect, the compositions are particularly useful in the treatment of tumors or cancerous cell that express or overexpress CLDN 18.2.

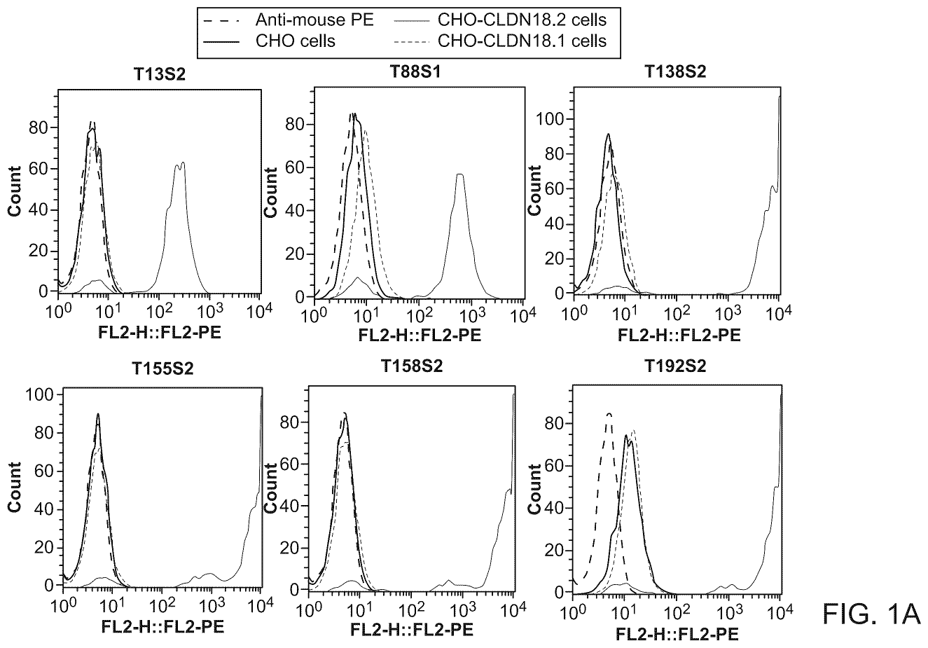

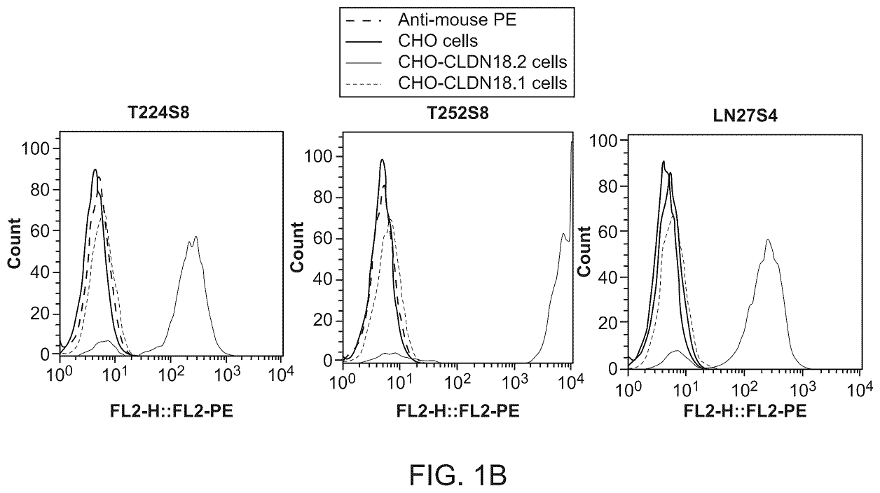

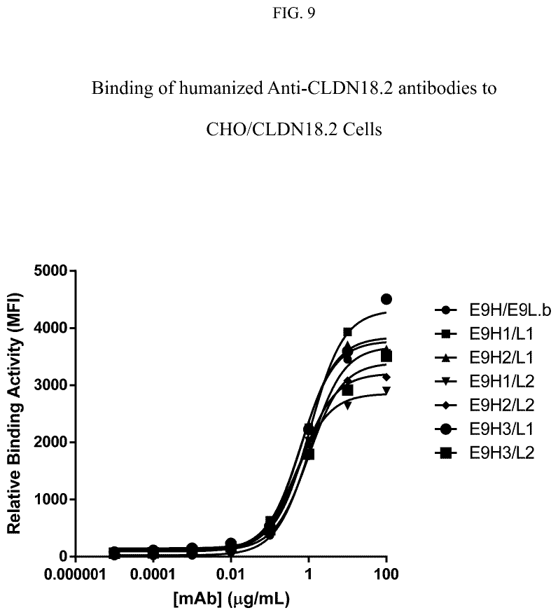

In one aspect, the present disclosure provides isolated antibodies, the antibodies comprising a heavy chain (HC) immunoglobulin variable domain sequence and a light chain (LC) immunoglobulin variable domain sequence, wherein the antibodies bind to an epitope of CLDN 18.2. In one aspect, the antibodies bind to an epitope of CLDN 18.2, but not CLDN 18.1. In one aspect, the antibody is a humanized antibody comprising one or more, two or more, three or more, four or more, five or more, six or more, seven or more or all eight of the CDRs identified in Table 3. In another aspect, the antibodies bind with high specificity, that is they bind to CLDN 18.2 with an affinity of more than 80%, or alternatively more than 85%, or alternatively more than 90%, or alternatively more than 95%, binding for CLDN18.2-expressing cells versus less than 20%, or alternatively less than 15%, or alternatively less than 10%, or alternatively less than 5% binding to CLDN 18.1. Example of such are shown in FIGS. 1a, 1b and 9. There, the antibodies are more specific for the CLDN18.2 expressing tumor cells and would have lower toxicity since they do not bind to the CLDN18.1-expressing normal cells.

In some embodiments, the antibody is a bispecific antibody has a binding specificity to a second target protein. The second target protein, in some embodiments, is selected from the group consisting of IL-1, CD3, CD16, CD19, CD28, CD47, CD64, PD-1, PD-L1, CTLA-4, LAG-3, CD28, CD122, 4-1BB, TIM3, OX-40, OX40L, CD40, CD40L, LIGHT, ICOS, ICOSL, GITR, GITRL, TIGIT, CD27, VISTA, B7H3, B7H4, HEVM, BTLA and KIR. In some embodiments, the antibody is comprised in an antibody drug conjugates (ADC) formed between the antibody and another agent, such as toxin or drug. In some embodiments, the antibodies may be conjugated to therapeutic agents, prodrugs, peptides, proteins, enzymes, viruses, lipids, biological response modifiers, pharmaceutical agents, and/or polyethylene glycol (PEG).

In a further aspect, this disclosure provides isolated anti-CLDN 18.2 antibodies or fragments thereof as disclosed herein and a detectable or purification label, alone or in combination with an CLDN 18.2 antigen or fragment (e.g., VH1, VH2, VH3, VL1, VL2, VL3, heavy and light chain CDR 1, CDR 2, heavy and light chain heavy and light chain CDR 1, CDR 2, CDR3) thereof, that in one aspect are bound to each other to form an antigen/antibody complex. Further provided herein is an ex vivo cell comprising this antigen/antibody complex.

Aspects of the disclosure relate to a chimeric antigen receptor (CAR) comprising: (a) an antigen binding domain of a CLDN 18.2 antibody (e.g., VH1, VH2, VH3, VL1, VL2, VL3, heavy and light chain CDR 1, CDR 2, CDR3); (b) a hinge domain; (c) a transmembrane domain; and (d) an intracellular domain. Further aspects of the disclosure relate to a chimeric antigen receptor (CAR) comprising: (a) an antigen binding domain of a CLDN 18.2 antibody (e.g., VH1, VH2, VH3, VL1, VL2, VL3, heavy and light chain CDR 1, CDR 2, CDR3); (b) a hinge domain; (c) a CD28 transmembrane domain; (d) one or more costimulatory regions selected from a CD28 costimulatory signaling region, a 4-1BB costimulatory signaling region, an ICOS costimulatory signaling region, and an OX40 costimulatory region; and (c) a CD3 zeta signaling domain and alternatives thereof.

In a further aspect, the present disclosure provides a chimeric antigen receptor (CAR) comprising: (a) an antigen binding domain of an anti-CLDN 18.2 antibody (e.g., VH1, VH2, VH3, VL1, VL2, VL3, heavy and light chain CDR 1, CDR 2, CDR3), (b) a CD8α hinge domain; (c) a CD8α transmembrane domain; (d) a CD28 and/or a 4-1BB costimulatory signaling region; and (e) a CD3 zeta signaling domain and alternatives thereof.

In another aspect, the present disclosure provides an isolated nucleic acid sequence encoding the anti-CLDN 18.2 antibody, humanized antibody, bispecific antibody, or the anti-CLDN 18.2 CAR.

In another aspect, the present disclosure provides a vector comprising the isolated nucleic acid sequence encoding the anti-CLDN 18.2 antibody, bispecific antibody, humanized antibody, or the anti-CLDN 18.2 CAR.

In another aspect, the present disclosure provides a vector comprising the isolated nucleic acid sequence encoding the anti-CLDN 18.2 antibody, bispecific antibody, humanized antibody, or the anti-CLDN 18.2 CAR, operatively linked to sequences that direct and optionally enhance the expression of the nucleic acid sequence. These sequences include promoters and enhancer sequences.

In another aspect, the present disclosure provides a composition comprising a carrier and one or more of: the anti-CLDN 18.2 antibody; and/or the chimeric antibody; and/or the bispecific antibody; and/or the humanized antibody; and/or the anti-CLDN 18.2 CAR; and/or the isolated nucleic acid encoding the anti-CLDN 18.2 antibody bispecific antibody, and/or the chimeric antibody; and/or the anti-CLDN 18.2 humanized antibody, or the anti-CLDN 18.2 CAR; and/or the vector comprising the isolated nucleic acid sequence encoding the anti-CLDN 18.2 antibody, bispecific antibody, and/or the chimeric antibody; and/or the humanized antibody, or the anti-CLDN 18.2 CAR; and/or an isolated cell comprising the anti-CLDN 18.2 CAR.

In one aspect, the disclosure provides a composition comprising, or alternatively consisting essentially of, or yet further consisting of a carrier and one or more of: an antibody or fragment (e.g., VH1, VH2, VH3, VL1, VL2, VL3, heavy and light chain CDR 1, CDR 2, CDR3) thereof, a nucleic acid encoding the antibody or fragment (e.g., VH1, VH2, VH3, VL1, VL2, VL3, heavy and light chain CDR 1, CDR 2, CDR3) thereof, a vecor comprising the nucleic acid encoding the antibody or fragment (e.g., VH1, VH2, VH3, VL1, VL2, VL3, heavy and light chain CDR 1, CDR 2, CDR3) thereof, an isolated cell comprising an anti-CLDN 18.2 CAR and/or a polynucleotide encoding the antibody or fragment (e.g., VH1, VH2, VH3, VL1, VL2, VL3, heavy and light chain CDR 1, CDR 2, CDR3) thereof; and/or the isolated nucleic acid encoding the CAR; and/or the vector comprising the nucleic acid encoding the CAR; and/or the isolated cell expressing an anti-CLDN 18.2 CAR; and/or the anti-CLDN 18.2 antibody; and/or the chimeric antibody; and/or bispecific antibody; and/or the humanized antibody.

Compositions are also provided. In one embodiment, the composition comprises an antibody or fragment (e.g., VH1, VH2, VH3, VL1, VL2, VL3, heavy and light chain CDR 1, CDR 2, CDR3) thereof, a fusion protein, or a CAR and/or car expressing-cell of the present disclosure and a pharmaceutically acceptable carrier. Also provided is an isolated cell comprising one or more polynucleotide encoding the antibody or fragment (e.g., VH1, VH2, VH3, VL1, VL2, VL3, heavy and light chain CDR 1, CDR 2, CDR3) thereof or the fusion protein or bispecific antibody of the present disclosure.

The present disclosure further provides a method of treating tumor or growth expressing CLND 18.2 antigen, such as for example cancer or fibrosis in a patient in need thereof, comprising administering to the patient a antibody or fragment (e.g., VH1, VH2, VH3, VL1, VL2, VL3, heavy and light chain CDR 1, CDR 2, CDR3) thereof, a fusion protein, or a CAR and/or car expressing-cell of the present disclosure. Non-limiting examples of cancers include GI cancers, bladder cancer, liver cancer, colon cancer, rectal cancer, endometrial cancer, biliary cancer, leukemia, lymphoma, pancreatic cancer, small cell lung cancer, non-small cell lung cancer, breast cancer, urethral cancer, head and neck cancer, gastrointestinal cancer, stomach cancer, oesophageal cancer, ovarian cancer, renal cancer, melanoma, prostate cancer and thyroid cancer. The methods can be combined with diagnostic methods to identify patients or subjects that are more likely to respond to the treatment.

### Antibodies and Uses Thereof

### I. Compositions

The general structure of antibodies is known in the art and will only be briefly summarized here. An immunoglobulin monomer comprises two heavy chains and two light chains connected by disulfide bonds. Each heavy chain is paired with one of the light chains to which it is directly bound via a disulfide bond. Each heavy chain comprises a constant region (which varies depending on the isotype of the antibody) and a variable region. The variable region comprises three hypervariable regions (or complementarity determining regions) which are designated CDRH1, CDRH2 and CDRH3 and which are supported within framework regions. Each light chain comprises a constant region and a variable region, with the variable region comprising three hypervariable regions (designated CDRL1, CDRL2 and CDRL3) supported by framework regions in an analogous manner to the variable region of the heavy chain.

The hypervariable regions of each pair of heavy and light chains mutually cooperate to provide an antigen binding site that is capable of binding a target antigen. The binding specificity of a pair of heavy and light chains is defined by the sequence of CDR1, CDR2 and CDR3 of the heavy and light chains. Thus once a set of CDR sequences (i.e. the sequence of CDR1, CDR2 and CDR3 for the heavy and light chains) is determined which gives rise to a particular binding specificity, the set of CDR sequences can, in principle, be inserted into the appropriate positions within any other antibody framework regions linked with any antibody constant regions in order to provide a different antibody with the same antigen binding specificity.

In one embodiment, the disclosure provides an isolated antibody comprising a heavy chain (HC) immunoglobulin variable domain sequence and a light chain (LC) immunoglobulin variable domain sequence, wherein the antibody binds to an epitope CLDN 18.2.

In one aspect, the HC of the antibody comprises or alternatively consists essentially of, or yet further consists of one or more of a CDR1 comprising the amino acid sequence selected from (i) GFTFSSYA (SEQ ID NO: 4), (ii) GFTFSDYY (SEQ ID NO: 10), (iii) GFNIKDYF (SEQ ID NO: 16), (iv) GFTFSDYY (SEQ ID NO: 10), (v) GYSFTGYN (SEQ ID NO: 28), (vi) GFSLTSYG (SEQ ID NO: 34), (vii) GYNMN (SEQ ID NO: 93 (or an equivalent of each thereof; and/or a CDR2 comprising the amino acid sequence selected from (i) ISGGGST (SEQ ID NO: 5), (ii) ISDGGGDT (SEQ ID NO: 11), (iii) IDPENGDT (SEQ ID NO: 17), (iv) ISNGGGST (SEQ ID NO: 23), (v) INPYNGGT (SEQ ID NO: 29), (vi) IWSDGRT (SEQ ID NO: 35), (vii) LINPYNGGTRYNQKFKG (SEQ ID NO: 94), or an equivalent of each thereof; and/or a CDR3 comprising the amino acid sequence selected from (i) ARGGNIYDGYPYYFDY (SEQ ID NO: 95), (ii) GGNIYDGYPYYFDY (SEQ ID NO: 6), (iii) ARHRGSLDY (SEQ ID NO: 12), (iv) NVLGYGNYGHFYYAMDY (SEQ ID NO: 18), (v) ARHRGSLDF (SEQ ID NO: 24), (vi) ARMGLGNAMDY (SEQ ID NO: 30), (vii) ARHGRYDPYAMDY (SEQ ID NO: 36), (viii) MGLGNAMDY (SEQ ID NO: 96), or an equivalent of each thereof; and/or the LC comprises the antibody of comprises or alternatively consists essentially of, or yet further consists of a CDR1 comprising the amino acid sequence selected from (i) QNLLNSSNQKNY (SEQ ID NO: 1), (ii) ESVDNYGFSF (SEQ ID NO: 7), (iii) QSLVHSNGNTY (SEQ ID NO: 13), (iv) QNLLNSGNQKNY (SEQ ID NO: 25), (v) KSSQNLLNSGNQKNYLT (SEQ ID NO: 97), or an equivalent of each thereof; and/or a CDR2 comprising the amino acid sequence selected from (i) FAS (SEQ ID NO: 2), (ii) RAS (SEQ ID NO: 8), (iii) KVS (SEQ ID NO: 14), (iv) WAS (SEQ ID NO: 26), (v) WASTMES (SEQ ID NO: 98), or an equivalent of each thereof; and/or a CDR3 comprising the amino acid sequence selected from (i) QQHYSTPLT (SEQ ID NO: 3), (ii) QQSNQGPLT (SEQ ID NO: 9), (iii) SQNTHVPRT (SEQ ID NO: 15), (iv) QQSNKVPLT (SEQ ID NO: 21), (v) QNDYIYPLP (SEQ ID NO: 27), or an equivalent of each thereof.

In one aspect, the HC of the antibody comprises or alternatively consists essentially of, or yet further consists of one or more of a CDR1 comprising the amino acid sequence of GFTFSSYA (SEQ ID NO: 4 (or an equivalent thereof; and/or a CDR2 comprising the amino acid sequence of ISGGGST (SEQ ID NO: 5 (or an equivalent thereof; and/or a CDR3 comprising the amino acid sequence of GGNIYDGYPYYFDY (SEQ ID NO: 6 (or an equivalent thereof; and/or the LC comprises the antibody of comprises or alternatively consists essentially of, or yet further consists of a CDR1 comprising the amino acid sequence of QNLLNSSNQKNY (SEQ ID NO: 1 (or an equivalent thereof; and/or a CDR2 comprising the amino acid sequence of FAS (SEQ ID NO: 2 (or an equivalent thereof; and/or a CDR3 comprising the amino acid sequence of QQHYSTPLT (SEQ ID NO: 3 (or an equivalent thereof.

In one aspect, the HC of the antibody comprises or alternatively consists essentially of, or yet further consists of one or more of a CDR1 comprising the amino acid sequence of GFTFSDYY (SEQ ID NO: 10 (or an equivalent thereof; and/or a CDR2 comprising the amino acid sequence of ISDGGGDT (SEQ ID NO: 11 (or an equivalent thereof; and/or a CDR3 comprising the amino acid sequence of ARHRGSLDY (SEQ ID NO: 12 (or an equivalent thereof; and/or the LC comprises the antibody of comprises or alternatively consists essentially of, or yet further consists of a CDR1 comprising the amino acid sequence of ESVDNYGFSF (SEQ ID NO: 7 (or an equivalent thereof; and/or a CDR2 comprising the amino acid sequence of RAS (SEQ ID NO: 8 (or an equivalent thereof; and/or a CDR3 comprising the amino acid sequence of QQSNQGPLT (SEQ ID NO: 9 (or an equivalent thereof.

In one aspect, the HC of the antibody comprises or alternatively consists essentially of, or yet further consists of one or more of a CDR1 comprising the amino acid sequence of GFNIKDYF (SEQ ID NO: 16 (or an equivalent thereof; and/or a CDR2 comprising the amino acid sequence of IDPENGDT (SEQ ID NO: 17 (or an equivalent thereof; and/or a CDR3 comprising the amino acid sequence of NVLGYGNYGHFYYAMDY (SEQ ID NO: 18 (or an equivalent thereof; and/or the LC comprises the antibody of comprises or alternatively consists essentially of, or yet further consists of a CDR1 comprising the amino acid sequence of QSLVHSNGNTY (SEQ ID NO: 13 (or an equivalent thereof; and/or a CDR2 comprising the amino acid sequence of KVS (SEQ ID NO: 14 (or an equivalent thereof; and/or a CDR3 comprising the amino acid sequence of SQNTHVPRT (SEQ ID NO: 15 (or an equivalent thereof.

In one aspect, the HC of the antibody comprises or alternatively consists essentially of, or yet further consists of one or more of a CDR1 comprising the amino acid sequence of GFTFSDYY (SEQ ID NO: 10 (or an equivalent thereof; and/or a CDR2 comprising the amino acid sequence of ISNGGGST (SEQ ID NO: 23 (or an equivalent thereof; and/or a CDR3 comprising the amino acid sequence of ARHRGSLDF (SEQ ID NO: 24 (or an equivalent thereof; and/or the LC comprises the antibody of comprises or alternatively consists essentially of, or yet further consists of a CDR1 comprising the amino acid sequence of ESVDNYGFSF (SEQ ID NO: 7 (or an equivalent thereof; and/or a CDR2 comprising the amino acid sequence of RAS (SEQ ID NO: 8 (or an equivalent thereof; and/or a CDR3 comprising the amino acid sequence of QQSNKVPLT (SEQ ID NO: 21 (or an equivalent thereof.

In one aspect, the HC of the antibody comprises or alternatively consists essentially of, or yet further consists of one or more of a CDR1 comprising the amino acid sequence of GYSFTGYN (SEQ ID NO: 28 (or an equivalent thereof; and/or a CDR2 comprising the amino acid sequence of INPYNGGT (SEQ ID NO: 29 (or an equivalent thereof; and/or a CDR3 comprising the amino acid sequence of ARMGLGNAMDY (SEQ ID NO: 30 (or an equivalent thereof; and/or the LC comprises the antibody of comprises or alternatively consists essentially of, or yet further consists of a CDR1 comprising the amino acid sequence of QNLLNSGNQKNY (SEQ ID NO: 25 (or an equivalent thereof; and/or a CDR2 comprising the amino acid sequence of WAS (SEQ ID NO: 26 (or an equivalent thereof; and/or a CDR3 comprising the amino acid sequence of QNDYIYPLP (SEQ ID NO: 27 (or an equivalent thereof.

In one aspect, the HC of the antibody comprises or alternatively consists essentially of, or yet further consists of one or more of a CDR1 comprising the amino acid sequence of GFSLTSYG (SEQ ID NO: 34 (or an equivalent thereof; and/or a CDR2 comprising the amino acid sequence of IWSDGRT (SEQ ID NO: 35 (or an equivalent thereof; and/or a CDR3 comprising the amino acid sequence of ARHGRYDPYAMDY (SEQ ID NO: 36 (or an equivalent thereof; and/or the LC comprises the antibody of comprises or alternatively consists essentially of, or yet further consists of a CDR1 comprising the amino acid sequence of ESVDNYGFSF (SEQ ID NO: 7 (or an equivalent thereof; and/or a CDR2 comprising the amino acid sequence of RAS (SEQ ID NO: 8 (or an equivalent thereof; and/or a CDR3 comprising the amino acid sequence of QQSNQGPLT (SEQ ID NO: 9 (or an equivalent thereof.

In one aspect, the HC of the antibody comprises or alternatively consists essentially of, or yet further consists of one or more of a CDR1 comprising the amino acid sequence of KSSQNLLNSGNQKNYLT (SEQ ID NO: 97 (or an equivalent thereof; and/or a CDR2 comprising the amino acid sequence of WASTMES (SEQ ID NO: 98 (or an equivalent thereof; and/or a CDR3 comprising the amino acid sequence of QNDYIYPLP (SEQ ID NO: 27 (or an equivalent thereof; and/or the LC comprises the antibody of comprises or alternatively consists essentially of, or yet further consists of a CDR1 comprising the amino acid sequence of GYNMN (SEQ ID NO: 93 (or an equivalent thereof; and/or a CDR2 comprising the amino acid sequence of LINPYNGGTRYNQKFKG (SEQ ID NO: 94 (or an equivalent thereof; and/or a CDR3 comprising the amino acid sequence of MGLGNAMDY (SEQ ID NO: 96 (or an equivalent thereof.

In one aspect, the heavy chain variable region comprises, or alternatively consists essentially of, or yet further consists of a heavy chain amino acid sequence of the anti-CLDN 18.2 antibody disclosed herein, and the light chain variable region comprises, or alternatively consists essentially of, or yet further consists of the light chain amino acid sequence from anti-CLDN 18.2 antibody disclosed herein, or equivalents thereof, as follows (CDRs are underlined):

### Anti-CLDN 18.2 Heavy Chain Amino Acid Sequence (T155S2):

(SEQ ID NO: 38(
EVKLVESGGGLVKPGGSLKLSCAASGFTFSSYAMSWVRQTPEKRLEWVAS
ISGGGSTYYPDSVKGRFTISRDNARNILYLQMSYLRSEDTAVYYCARGGN
IYDGYPYYFDYWGQGTTLTVSS,
or an equivalent thereof.

### Anti-CLDN 18.2 Light Chain Amino Acid Sequence (T155S2):

(SEQ ID NO: 40(
DIVMTQSPSSLAMSVGQKVTMSCKSSQNLLNSSNQKNYLAWYQQKPGQSP
KLLVYFASTRESGVPDRFTGSGSGTDFTLTISSVQAEDLAVYYCQQHYST
PLTFGAGTKLELK,
or an equivalent thereof.

### Anti-CLDN 18.2 Heavy Chain Amino Acid Sequence (T158S2):

(SEQ ID NO: 42(
EVKLVESGGGLVQPGGSLKLSCATSGFTFSDYYMFWIRQTPEKRLEWVAS
ISDGGGDTYYPDTVKGRFTISRDNAKNTLYLQMSRLKSEDTAMYYCARHR
GSLDYWGQGTTLTVSS,
or an equivalent thereof.

### Anti-CLDN 18.2 Light Chain Amino Acid Sequence (T158S2):

(SEQ ID NO: 44(
DIVLTQSPASLAVSLGQRATISCRASESVDNYGFSFLHWYQQKPGQPPKL
LIYRASNLASGIPARFSGSGSRTDFTLTINPVETDDVATYYCQQSNQGPL
TFGAGTKLELK,
or an equivalent thereof.

### Anti-CLDN 18.2 Heavy Chain Amino Acid Sequence (T224S8):

(SEQ ID NO: 46(
EVQLQQSGTELVRSGASVKLSCTTSGFNIKDYFLHWVKQRPEQGLEWIGW
IDPENGDTKYAPKFQDKVTMTVDTSSNTACLHLSSLTSDDTAVYYCNVLG
YGNYGHFYYAMDYWGQGTSVTVSS,
or an equivalent thereof.

### Anti-CLDN 18.2 Light Chain Amino Acid Sequence (T224S8):

(SEQ ID NO: 48(
DVVMTQTPLSLPVSLGDQASISCRSSQSLVHSNGNTYLHWYLQKPGQSPK
LLIYKVSNRFSGVPDRFSGSESGTDFTLKISRVEAEDLGIYFCSQNTHVP
RTFGGGTKLEIR,
or an equivalent thereof.

### Anti-CLDN 18.2 Heavy Chain Amino Acid Sequence (T192S2):

(SEQ ID NO: 50(
EVKLVESGGGLVQPGGSLKLSCATSGFTFSDYYMYWVRQTPEKRLEWVAY
ISNGGGSTYYPDTVKGRFTISRDNAKNTLYLQMSRLKSEDTAMYYCARHR
GSLDFWGQGTTLTVSS,
or an equivalent thereof.

### Anti-CLDN 18.2 Light Chain Amino Acid Sequence (T192S2):

(SEQ ID NO: 52(
DIVLTQSPASLAVSLGQRATISCRASESVDNYGFSFMHWYQQKPGQPPK
LLIYRASNLESGIPARFSGSWSRTDFTLTINPVETDDVATYYCQQSNKV
PLTFGAGTKLELK, or an equivalent thereof.

### Anti-CLDN 18.2 Heavy Chain Amino Acid Sequence (T252S4):

(SEQ ID NO: 54(
EVQLQQSGPELVKPGASMKISCKASGYSFTGYNMNWVKQTHGKNLEWIG
LINPYNGGTRYNQKFKGKATLTVDKSSSTAYMELLSLTSEDSAVYFCAR
MGLGNAMDYWGQGTSVTVSS, or an equivalent thereof.

### Anti-CLDN 18.2 Light Chain Amino Acid Sequence (T252S4):

(SEQ ID NO: 56(
DIVMTQSPSSLTVTAGEKVTMSCKSSQNLLNSGNQKNYLTWYQQKPGQP
PKLLIYWASTMESGVPDRFTGSGSGTDFTLTISSVQAEDLAVYYCQNDY
IYPLPFGAGTKLELK, or an equivalent thereof.

### Anti-CLDN 18.2 Heavy Chain Amino Acid Sequence (T88S1):

(SEQ ID NO: 58(
QVQLKESGPGLVAPSQSLSITCTISGFSLTSYGIHWVRQPPGKGLEWLV
VIWSDGRTTYNSALKSRLSISKDNSKSQVFLKMNSLQTDDTAMYYCARH
GRYDPYAMDYWGQGTSVTVSS, or an equivalent thereof.

### Anti-CLDN 18.2 Light Chain Amino Acid Sequence (T88S1):

(SEQ ID NO: 44(
DIVLTQSPASLAVSLGQRATISCRASESVDNYGFSFLHWYQQKPGQPPK
LLIYRASNLASGIPARFSGSGSRTDFTLTINPVETDDVATYYCQQSNQG
PLTFGAGTKLELK, or an equivalent thereof.

### Anti-CLDN 18.2 Heavy Chain Amino Acid Sequence (Chimeric T252S4):

(SEQ ID NO: 54(
EVQLQQSGPELVKPGASMKISCKASGYSFTGYNMNWVKQTHGKNLEWIG
LINPYNGGTRYNQKFKGKATLTVDKSSSTAYMELLSLTSEDSAVYFCAR
MGLGNAMDYWGQGTSVTVSS, or an equivalent thereof.

### Anti-CLDN 18.2 Light Chain Amino Acid Sequence (Chimeric T252S4):

(SEQ ID NO: 56(
DIVMTQSPSSLTVTAGEKVTMSCKSSQNLLNSGNQKNYLTWYQQKPGQP
PKLLIYWASTMESGVPDRFTGSGSGTDFTLTISSVQAEDLAVYYCQNDY
IYPLPFGAGTKLELK, or an equivalent thereof.

### Anti-CLDN 18.2 Heavy Chain Amino Acid Sequence (HuVH1 T252S4):

(SEQ ID NO: 62(
EVQLVQSGAEVKKPGESLKISCKGSGYSFTGYNMNWVRQMPGKGLEWMG
LINPYNGGTRYNQKFKGQVTISADKSISTAYLQWSSLKASDTAMYFCAR
MGLGNAMDYWGQGTTVTVSS, or an equivalent thereof.

### Anti-CLDN 18.2 Heavy Chain Amino Acid Sequence (HuVH2 T252S4):

(SEQ ID NO: 63(
EVQLVQSGAEVKKPGESLKISCKGSGYSFTGYNMNWVRQMPGKNLEWIG
LINPYNGGTRYNQKFKGQVTISADKSISTAYLQWSSLKASDTAMYFCAR
MGLGNAMDYWGQGTTVTVSS, or an equivalent thereof.

### Anti-CLDN 18.2 Heavy Chain Amino Acid Sequence (HuVH3 T252S4):

(SEQ ID NO: 64(
EVQLVQSGAEVKKPGESLKISCKGSGYSFTGYNMNWVRQMPGKNLEWIG
LINPYNGGTRYNQKFKGKATLSVDKSISTAYLQWSSLKASDTAMYFCAR
MGLGNAMDYWGQGTTVTVSS, or an equivalent thereof.

### Anti-CLDN 18.2 Light Chain Amino Acid Sequence (HuVL1 T252S4):

(SEQ ID NO: 66(
DIVMTQSPDSLAVSLGERATINCKSSQNLLNSGNQKNYLTWYQQKPGQP
PKLLIYWASTMESGVPDRFSGSGSGTDFTLTISSLQAEDVAVYYCQNDY
IYPLPFGQGTKLEIK, or an equivalent thereof.

### Anti-CLDN 18.2 Light Chain Amino Acid Sequence (HuVL2 T252S4):

(SEQ ID NO: 67)
DIVMTQSPDSLAVSLGERATINCKSSQNLLQSGNQKNYLTWYQQKPGQP
PKLLIYWASTRESGVPDRFSGSGSGTDFTLTISSLQAEDVAVYYCQNDY
IYPLPFGQGTKLEIK, or an equivalent thereof.

In one aspect, the heavy chain variable region comprises, or alternatively consists essentially of, or yet further consists of an heavy chain encoded by the nucleic acid sequence of the anti-CLDN 18.2 antibody disclosed herein, and the light chain variable region comprises, or alternatively consists essentially of, or yet further consists of the light chain chain encoded by the nucleic acid sequence from anti-CLDN 18.2 antibody disclosed herein, or equivalents thereof, as follows (CDRs are underlined):

### Anti-CLDN 18.2 Heavy Chain Nucleic Acid Sequence (T155S2):

(SEQ ID NO: 37(
GAAGTGAAGCTGGTGGAGTCTGGGGGAGGCTTAGTGAAGCCTGGAGGGT
CCCTGAAACTCTCCTGTGCAGCCTCTGGATTCACTTTCAGTAGCTATGC
CATGTCTTGGGTTCGCCAGACTCCAGAGAAGAGGCTGGAGTGGGTCGCA
TCCATTAGTGGTGGTGGTAGCACCTACTATCCAGACAGTGTGAAGGGCC
GATTCACCATCTCCAGAGATAATGCCAGGAACATCCTGTACCTGCAAAT
GAGCTATCTGAGGTCTGAGGACACGGCCGTGTATTACTGTGCAAGAGGA
GGGAATATCTATGATGGTTACCCGTACTACTTTGACTACTGGGGCCAAG
GCACCACTCTCACAGTCTCCTCA, or an equivalent thereof.

### Anti-CLDN 18.2 Light Chain Nucleic Acid Sequence (T155S2):

(SEQ ID NO: 39(
GACATTGTGATGACCCAGTCTCCATCCTCCCTGGCTATGTCAGTAGGAC
AGAAGGTCACTATGAGCTGCAAGTCCAGTCAGAACCTTTTAAATAGTAG
CAATCAAAAGAACTATTTGGCCTGGTACCAGCAGAAACCAGGACAGTCT
CCTAAACTTCTGGTATACTTTGCATCCACTAGGGAATCTGGGGTCCCTG
ATCGCTTCACAGGCAGTGGATCTGGGACAGATTTCACTCTTACCATCAG
CAGTGTGCAGGCTGAAGACCTGGCAGTTTATTACTGTCAGCAACATTAT
AGCACTCCGCTCACGTTCGGTGCTGGGACCAAGCTGGAGCTGAAA,
or an equivalent thereof.

### Anti-CLDN 18.2 Heavy Chain Nucleic Acid Sequence (T158S2):

(SEQ ID NO: 41(
GAAGTGAAGCTGGTGGAGTCTGGGGGAGGCTTAGTGCAGCCTGGAGGGT
CCCTGAAACTCTCCTGTGCAACCTCTGGATTCACTTTCAGTGACTATTA
CATGTTTTGGATTCGCCAGACTCCAGAGAAGAGGCTGGAGTGGGTCGCC
TCCATTAGTGATGGTGGTGGTGACACCTATTATCCAGACACTGTAAAGG
GCCGATTCACCATCTCCAGAGACAATGCCAAGAACACCCTGTACCTGCA
AATGAGCCGTCTGAAGTCTGAGGACACAGCCATGTATTACTGTGCAAGA
CATAGGGGCTCTCTTGACTACTGGGGCCAAGGCACCACTCTCACAGTCT
CCTCA, or an equivalent thereof.

### Anti-CLDN 18.2 Light Chain Nucleic Acid Sequence (T158S2):

(SEQ ID NO: 43(
GACATTGTGCTGACCCAATCTCCAGCTTCTTTGGCTGTGTCTCTAGGGCA
GAGGGCCACCATCTCCTGCAGAGCCAGCGAGAGTGTGGATAACTATGGCT
TTAGTTTTCTGCACTGGTACCAGCAGAAACCAGGACAGCCACCCAAACTC
CTCATCTATCGTGCATCCAACCTAGCATCTGGGATCCCTGCCAGGTTCAG
TGGCAGTGGGTCTAGGACAGACTTCACCCTCACCATTAATCCTGTGGAGA
CTGATGATGTTGCAACCTATTACTGTCAGCAAAGTAATCAGGGTCCACTC
ACGTTCGGTGCTGGGACCAAGCTGGAGCTGAAA,
or an equivalent thereof.

### Anti-CLDN 18.2 Heavy Chain Nucleic Acid Sequence (T224S8):

(SEQ ID NO: 45(
GAGGTTCAGCTGCAGCAGTCTGGGACAGAGCTTGTGAGGTCAGGGGCCTC
AGTCAAGTTGTCCTGCACAACTTCTGGCTTCAACATTAAAGACTACTTTT
TACACTGGGTGAAGCAGAGGCCTGAACAGGGCCTGGAGTGGATTGGATGG
ATTGATCCTGAGAATGGTGATACTAAATATGCCCCGAAGTTCCAGGACAA
GGTCACCATGACTGTGGACACATCCTCCAACACAGCCTGCCTGCACCTCA
GCAGCCTGACATCTGATGACACTGCCGTCTATTACTGTAATGTACTAGGC
TACGGTAATTACGGACATTTTTACTATGCTATGGACTACTGGGGTCAAGG
AACCTCAGTCACCGTCTCCTCT,
or an equivalent thereof.

### Anti-CLDN 18.2 Light Chain Nucleic Acid Sequence (T224S8):

(SEQ ID NO: 47(
GATGTTGTGATGACCCAAACTCCACTCTCCCTGCCTGTCAGTCTTGGAGA
TCAGGCCTCCATCTCTTGCAGATCTAGTCAGAGCCTTGTACACAGTAATG
GAAACACCTATTTACATTGGTACCTGCAGAAGCCAGGCCAGTCTCCAAAG
CTCCTGATCTACAAAGTTTCCAACCGATTTTCTGGGGTCCCAGACAGGTT
CAGTGGCAGTGAATCAGGGACAGATTTCACACTCAAGATCAGCAGAGTGG
AGGCTGAGGATCTGGGAATTTATTTCTGCTCTCAAAATACACATGTTCCT
CGGACGTTCGGTGGAGGCACCAAGCTGGAAATCAGA,
or an equivalent thereof.

### Anti-CLDN 18.2 Heavy Chain Nucleic Acid Sequence (T192S2):

(SEQ ID NO: 49(
GAAGTGAAGCTGGTGGAGTCTGGGGGAGGCTTAGTGCAGCCTGGAGGGTC
CCTGAAACTCTCCTGTGCAACCTCTGGATTCACTTTCAGTGACTATTACA
TGTATTGGGTTCGCCAGACTCCAGAGAAGAGGCTGGAGTGGGTCGCATAC
ATTAGTAATGGTGGTGGTTCCACCTATTATCCAGACACTGTGAAGGGCCG
ATTCACCATCTCCAGAGACAATGCCAAGAACACCCTGTACCTGCAAATGA
GCCGTCTGAAGTCTGAGGACACAGCCATGTATTACTGTGCAAGACATAGG
GGCTCTCTTGACTTCTGGGGCCAAGGCACCACTCTCACAGTCTCCTCA,
or an equivalent thereof.

### Anti-CLDN 18.2 Light Chain Nucleic Acid Sequence (T192S2):

(SEQ ID NO: 51(
GACATAGTGCTGACACAGTCTCCTGCTTCCTTAGCTGTATCTCTGGGGCA
GAGGGCCACCATCTCCTGCAGAGCCAGCGAAAGTGTTGATAATTATGGCT
TTAGTTTTATGCACTGGTACCAGCAGAAACCAGGACAGCCACCCAAACTC
CTCATCTATCGTGCATCCAACCTAGAATCTGGGATCCCTGCCAGGTTCAG
TGGCAGTTGGTCTAGGACAGACTTCACCCTCACCATTAATCCTGTGGAGA
CTGATGATGTTGCAACCTATTACTGTCAGCAAAGTAATAAGGTTCCGCTC
ACGTTCGGTGCTGGGACCAAGCTGGAGCTGAAA,
or an equivalent thereof.

### Anti-CLDN 18.2 Heavy Chain Nucleic Acid Sequence (T252S4):

(SEQ ID NO: 53(
GAGGTCCAGCTGCAACAGTCTGGACCTGAGCTGGTGAAGCCTGGAGCTTC
AATGAAGATATCCTGCAAGGCTTCTGGTTACTCATTCACTGGCTACAACA
TGAACTGGGTGAAGCAGACCCATGGAAAGAACCTTGAGTGGATTGGACTT
ATTAATCCTTACAATGGTGGTACTAGGTACAACCAGAAGTTCAAGGGCAA
GGCCACATTAACTGTAGACAAGTCATCCAGCACAGCCTACATGGAGCTCC
TCAGTCTGACATCTGAGGACTCTGCAGTCTATTTCTGTGCAAGGATGGGA
CTTGGAAATGCTATGGACTACTGGGGTCAAGGAACCTCAGTCACCGTCTC
CTCA,
or an equivalent thereof.

### Anti-CLDN 18.2 Light Chain Nucleic Acid Sequence (T252S4):

(SEQ ID NO: 55(
GACATTGTGATGACACAGTCTCCATCCTCCCTGACTGTGACAGCAGGAGA
GAAGGTCACTATGAGCTGCAAGTCCAGTCAGAATCTGTTAAACAGTGGAA
ATCAAAAGAACTACTTGACCTGGTACCAGCAGAAACCAGGGCAGCCTCCT
AAACTGTTGATCTACTGGGCATCCACTATGGAATCTGGGGTCCCTGATCG
CTTCACAGGCAGTGGATCTGGAACAGATTTCACTCTCACCATCAGCAGTG
TGCAGGCTGAAGACCTGGCAGTTTATTACTGTCAGAATGATTATATTTAT
CCGCTCCCGTTCGGTGCTGGGACCAAGCTGGAGCTGAAA,
or an equivalent thereof.

### Anti-CLDN 18.2 Heavy Chain Nucleic Acid Sequence (T88S1):

(SEQ ID NO: 57(
CAGGTGCAGCTGAAGGAGTCAGGACCTGGCCTGGTGGCGCCCTCACAGAG
CCTGTCCATCACATGCACCATCTCAGGGTTCTCATTAACCAGCTATGGTA
TACACTGGGTTCGCCAGCCTCCAGGAAAGGGTCTGGAGTGGCTGGTAGTG
ATATGGAGTGATGGAAGAACAACCTATAATTCAGCTCTCAAATCCAGACT
GAGCATCAGCAAGGACAACTCCAAGAGCCAAGTTTTCTTAAAAATGAACA
GTCTCCAAACTGATGACACAGCCATGTACTACTGTGCCAGACATGGTAGA
TACGACCCCTATGCTATGGACTACTGGGGTCAAGGAACCTCAGTCACCGT
CTCCTCA,
or an equivalent thereof.

### Anti-CLDN 18.2 Light Chain Nucleic Acid Sequence (T88S1):

(SEQ ID NO: 43(
GACATTGTGCTGACCCAATCTCCAGCTTCTTTGGCTGTGTCTCTAGGGC
AGAGGGCCACCATCTCCTGCAGAGCCAGCGAGAGTGTGGATAACTATGGC
TTTAGTTTTCTGCACTGGTACCAGCAGAAACCAGGACAGCCACCCAAACT
CCTCATCTATCGTGCATCCAACCTAGCATCTGGGATCCCTGCCAGGTTCA
GTGGCAGTGGGTCTAGGACAGACTTCACCCTCACCATTAATCCTGTGGAG
ACTGATGATGTTGCAACCTATTACTGTCAGCAAAGTAATCAGGGTCCACT
CACGTTCGGTGCTGGGACCAAGCTGGAGCTGAAA,
or an equivalent thereof.

### Anti-CLDN 18.2 Chimeric Heavy Chain Nucleic Acid Sequence (T252S4):

(SEQ ID NO: 53(
GAGGTCCAGCTGCAACAGTCTGGACCTGAGCTGGTGAAGCCTGGAGCTTC
AATGAAGATATCCTGCAAGGCTTCTGGTTACTCATTCACTGGCTACAACA
TGAACTGGGTGAAGCAGACCCATGGAAAGAACCTTGAGTGGATTGGACTT
ATTAATCCTTACAATGGTGGTACTAGGTACAACCAGAAGTTCAAGGGCAA
GGCCACATTAACTGTAGACAAGTCATCCAGCACAGCCTACATGGAGCTCC
TCAGTCTGACATCTGAGGACTCTGCAGTCTATTTCTGTGCAAGGATGGGA
CTTGGAAATGCTATGGACTACTGGGGTCAAGGAACCTCAGTCACCGTCTC
CTCA

### Anti-CLDN 18.2 Humanized Heavy Chain Nucleic Acid Sequence 1 (Hu VH1 T252S4):

(SEQ ID NO: 69(
GAAGTCCAGCTCGTGCAGTCTGGTGCGGAGGTCAAAAAACCCGGCGAGTC
TCTCAAAATTAGTTGCAAAGGCTCCGGTTATTCATTCACGGGTTACAATA
TGAACTGGGTTCGACAAATGCCGGGTAAGGGACTTGAATGGATGGGATTG
ATCAACCCCTATAACGGAGGTACCAGATACAACCAAAAATTTAAGGGCCA
GGTGACTATTAGTGCAGATAAAAGTATCTCCACCGCTTACCTCCAATGGT
CTAGTTTGAAGGCGTCTGACACTGCTATGTACTTTTGCGCGAGAATGGGG
TTGGGAAATGCGATGGACTACTGGGGTCAGGGCACTACGGTTACGGTCTC
CTCT

### Anti-CLDN 18.2 Humanized Heavy Chain Nucleic Acid Sequence 2 (Hu VH2 T252S4):

(SEQ ID NO: 70(
GAGGTACAGCTCGTGCAATCTGGGGCCGAAGTAAAGAAACCTGGAGAAAG
TCTTAAAATAAGCTGCAAGGGCAGTGGGTATAGCTTCACGGGCTACAATA
TGAACTGGGTTAGACAAATGCCAGGTAAGAACCTCGAATGGATAGGATTG
ATCAACCCATACAATGGTGGTACGCGCTACAACCAGAAATTCAAAGGTCA
GGTTACCATCTCTGCGGATAAAAGCATCTCAACGGCGTATCTTCAATGGT
CCTCACTGAAAGCATCCGACACAGCAATGTATTTTTGCGCTAGAATGGGA
TTGGGTAATGCCATGGACTACTGGGGCCAAGGCACCACGGTTACAGTCTC
TTCC

### Anti-CLDN 18.2 Humanized Heavy Chain Nucleic Acid Sequence 3 (Hu VH3 T252S4):

(SEQ ID NO: 71(
GAAGTTCAGCTGGTGCAAAGTGGGGCTGAGGTAAAGAAGCCTGGCGAGAG
CCTCAAGATTTCTTGCAAGGGTAGCGGATACAGTTTTACGGGATATAACA
TGAATTGGGTTCGCCAGATGCCGGGGAAGAACCTTGAATGGATAGGACTG
ATAAACCCCTATAATGGGGGAACCCGATATAATCAGAAGTTTAAGGGAAA
AGCAACTTTGTCAGTTGACAAGTCTATCAGCACGGCCTATCTTCAGTGGT
CCAGTCTGAAAGCAAGCGACACGGCTATGTACTTTTGTGCACGCATGGGG
CTTGGTAACGCAATGGACTATTGGGGACAAGGAACTACCGTCACTGTCTC
TTCA

### Anti-CLDN 18.2 Chimeric Light Chain Nucleic Acid Sequence 3 (Chimeric VL T252S4):

(SEQ ID NO: 55(
GACATTGTGATGACACAGTCTCCATCCTCCCTGACTGTGACAGCAGGAGA
GAAGGTCACTATGAGCTGCAAGTCCAGTCAGAATCTGTTAAACAGTGGAA
ATCAAAAGAACTACTTGACCTGGTACCAGCAGAAACCAGGGCAGCCTCCT
AAACTGTTGATCTACTGGGCATCCACTATGGAATCTGGGGTCCCTGATCG
CTTCACAGGCAGTGGATCTGGAACAGATTTCACTCTCACCATCAGCAGTG
TGCAGGCTGAAGACCTGGCAGTTTATTACTGTCAGAATGATTATATTTAT
CCGCTCCCGTTCGGTGCTGGGACCAAGCTGGAGCTGAAA

### Anti-CLDN 18.2 Humanized Light Chain Nucleic Acid Sequence 1 (Hu VL1 T252S4):

(SEQ ID NO: 73(
GACATCGTGATGACCCAGTCCCCGGACAGTCTGGCAGTCAGTCTTGGGGA
AAGAGCTACCATAAACTGCAAATCCAGCCAAAACCTTCTTAATAGCGGCA
ACCAAAAGAATTACTTGACTTGGTATCAGCAAAAACCGGGTCAGCCGCCC
AAACTCTTGATATACTGGGCGTCTACGATGGAAAGCGGCGTCCCCGACCG
CTTCAGCGGGAGTGGGTCAGGGACTGATTTCACTTTGACAATCAGTTCCC
TTCAGGCAGAGGACGTAGCAGTCTACTACTGTCAGAATGATTATATATAT
CCTCTTCCGTTCGGCCAGGGGACGAAGTTGGAGATCAAA

### Anti-CLDN 18.2 Humanized Light Chain Nucleic Acid Sequence 3 (Hu VL2 T252S4):

(SEQ ID NO: 74(
GACATCGTGATGACTCAAAGTCCTGACTCCCTTGCTGTTTCACTTGGCGA
AAGGGCCACTATCAACTGTAAGAGTTCTCAGAATCTCTTGCAATCAGGAA
ACCAGAAGAATTACTTGACCTGGTATCAACAGAAGCCTGGACAACCACCT
AAGCTCTTGATTTACTGGGCTAGTACAAGGGAGTCCGGCGTCCCAGACAG
ATTTTCCGGTTCTGGATCAGGCACGGACTTCACTCTGACAATCTCTAGTC
TTCAAGCCGAGGATGTGGCCGTTTATTATTGCCAGAACGATTACATTTAC
CCTTTGCCATTTGGTCAAGGTACTAAGTTGGAGATAAAA

In one aspect, humanized antibodies having the above-identified VH and VL combinations also are provided in Table 4.

In one aspect, the disclosure provides an isolated anti-CLDN 18.2 antibody which is generated against a CLDN 18.2 fragment.

In another aspect of the present technology, the isolated antibody includes one or more of the following characteristics:
(a) the light chain immunoglobulin variable domain sequence comprises one or more CDRs that are at least 85% identical to a CDR of a light chain variable domain of any of the disclosed light chain sequences;
(b) the heavy chain immunoglobulin variable domain sequence comprises one or more CDRs that are at least 85% identical to a CDR of a heavy chain variable domain of any of the disclosed heavy chain sequences;
(c) the light chain immunoglobulin variable domain sequence is at least 85% identical to a light chain variable domain of any of the disclosed light chain sequences;
(d) the HC immunoglobulin variable domain sequence is at least 85% identical to a heavy chain variable domain of any of the disclosed light chain sequences; and
(e) the antibody binds an epitope that overlaps with an epitope bound by any of the disclosed sequences.

In one aspect, the present disclosure provides an isolated antibody that is at least 85% identical to the anti-CLDN 18.2 antibody disclosed herein.

In some of the aspects of the antibodies provided herein, the antibody binds human CLDN 18.2 with a dissociation constant (K_{D}) of less than 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, or 10⁻¹² M. In some of the aspects of the antibodies provided herein, the antigen binding site specifically binds to human CLDN 18.2.

In some of the aspects of the antibodies provided herein, the antibody is soluble Fab.

In some of the aspects of the antibodies provided herein, the HC and LC variable domain sequences are components of the same polypeptide chain. In some of the aspects of the antibodies provided herein, the HC and LC variable domain sequences are components of different polypeptide chains.

In another embodiment, the antibody is a monoclonal antibody comprising an anti-CLDN 18.2 heavy chain variable region comprising, or alternatively consisting essentially of, or yet further consisting of a polypeptide selected disclosed herein or an equivalent of each thereof, and an anti-CLDN 18.2 light chain variable region comprising, or alternatively consisting essentially of, or yet further consisting of a polypeptide disclosed herein or an equivalent of each thereof.

In another aspect, the anti-CLDN 18.2 antibody is a chimeric antibody, human or a humanized antibody.

In some of the aspects of the antibodies provided herein, the antibody is a monoclonal antibody.

In some of the aspects of the antibodies provided herein, the antibody is a full-length antibody. In other aspect, antigen binding fragments of the antibodies are provided.

In some of the aspects of the antibodies provided herein, the antibody fragment is selected from the group consisting of Fab, F(ab)′2, Fab′, scFᵥ, and Fv.

In other aspects, one or more amino acid residues in a CDR of the antibodies provided herein are substituted with another amino acid. The substitution may be “conservative” in the sense of being a substitution within the same family of amino acids. The naturally occurring amino acids may be divided into the following four families and conservative substitutions will take place within those families:
1) Amino acids with basic side chains: lysine, arginine, histidine;
2) Amino acids with acidic side chains: aspartic acid, glutamic acid;
3) Amino acids with uncharged polar side chains: asparagine, glutamine, serine, threonine, tyrosine;
4) Amino acids with nonpolar side chains: glycine, alanine, valine, leucine, isoleucine, proline, phenylalanine, methionine, tryptophan, cysteine.

In another aspect, one or more amino acid residues are added to or deleted from one or more CDRs of an antibody. Such additions or deletions occur at the N or C termini of the CDR or at a position within the CDR.

By varying the amino acid sequence of the CDRs of an antibody by addition, deletion or substitution of amino acids, various effects such as increased binding affinity for the target antigen may be obtained.

It is to be appreciated that antibodies of the present disclosure comprising such varied CDR sequences still bind CLDN 18.2 with similar specificity and sensitivity profiles as the disclosed antibodies. This may be tested by way of the binding assays.

The constant regions of antibodies may also be varied. For example, antibodies may be provided with Fc regions of any isotype: IgA (IgA1, IgA2), IgD, IgE, IgG (IgG1, IgG2, IgG3, IgG4) or IgM.

### Human IgD Constant Region, Uniprot: P01880

(SEQ ID NO: 99(
APTKAPDVFPIISGCRHPKDNSPVVLACLITGYHPTSVTVTWYMGTQSQP
QRTFPEIQRRDSYYMTSSQLSTPLQQWRQGEYKCVVQHTASKSKKEIFRW
PESPKAQASSVPTAQPQAEGSLAKATTAPATTRNTGRGGEEKKKEKEKEE
QEERETKTPECPSHTQPLGVYLLTPAVQDLWLRDKATFTCFVVGSDLKDA
HLTWEVAGKVPTGGVEEGLLERHSNGSQSQHSRLTLPRSLWNAGTSVTCT
LNHPSLPPQRLMALREPAAQAPVKLSLNLLASSDPPEAASWLLCEVSGFS
PPNILLMWLEDQREVNTSGFAPARPPPQPGSTTFWAWSVLRVPAPPSPQP
ATYTCVVSHEDSRTLLNASRSLEVSYVTDHGPMK,
or an equivalent thereof.

### Human IgG1 Constant Region, Uniprot: P01857

(SEQ ID NO: 100(
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK,
or an equivalent thereof.

### Human IgG2 Constant Region, Uniprot: P01859

(SEQ ID NO: 101(
ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSHEDP
EVQFNWYVDGVEVHNAKTKPREEQFNSTFRVVSVLTVVHQDWLNGKEYKC
KVSNKGLPAPIEKTISKTKGQPREPQVYTLPPSREEMTKNQVSLTCLVKG
FYPSDISVEWESNGQPENNYKTTPPMLDSDGSFFLYSKLTVDKSRWQQGN
VFSCSVMHEALHNHYTQKSLSLSPGK,
or an equivalent thereof.

### Human IgG3 Constant Region, Uniprot: P01860

(SEQ ID NO: 102(
ASTKGPSVFPLAPCSRSTSGGTAALGCLVKDYFPEPVTVSWNSGALTSGV
HTFPAVLQSSGLYSLSSVVTVPSSSLGTQTYTCNVNHKPSNTKVDKRVEL
KTPLGDTTHTCPRCPEPKSCDTPPPCPRCPEPKSCDTPPPCPRCPEPKSC
DTPPPCPRCPAPELLGGPSVFLFPPKPKDTLMISRTPEVTCVVVDVSHED
PEVQFKWYVDGVEVHNAKTKPREEQYNSTFRVVSVLTVLHQDWLNGKEYK
CKVSNKALPAPIEKTISKTKGQPREPQVYTLPPSREEMTKNQVSLTCLVK
GFYPSDIAVEWESSGQPENNYNTTPPMLDSDGSFFLYSKLTVDKSRWQQG
NIFSCSVMHEALHNRFTQKSLSLSPGK,
or an equivalent thereof.

### Human IgM Constant Region, Uniprot: P01871

(SEQ ID NO: 103(
GSASAPTLFPLVSCENSPSDTSSVAVGCLAQDFLPDSITLSWKYKNNSDI
SSTRGFPSVLRGGKYAATSQVLLPSKDVMQGTDEHVVCKVQHPNGNKEKN
VPLPVIAELPPKVSVFVPPRDGFFGNPRKSKLICQATGFSPRQIQVSWLR
EGKQVGSGVTTDQVQAEAKESGPTTYKVTSTLTIKESDWLGQSMFTCRVD
HRGLTFQQNASSMCVPDQDTAIRVFAIPPSFASIFLTKSTKLTCLVTDLT
TYDSVTISWTRONGEAVKTHTNISESHPNATFSAVGEASICEDDWNSGER
FTCTVTHTDLPSPLKQTISRPKGVALHRPDVYLLPPAREQLNLRESATIT
CLVTGFSPADVFVQWMQRGQPLSPEKYVTSAPMPEPQAPGRYFAHSILTV
SEEEWNTGETYTCVAHEALPNRVTERTVDKSTGKPTLYNVSLVMSDTAGT
CY,
or an equivalent thereof.

### Human IgG4 Constant Region, Uniprot: P01861

(SEQ ID NO: 104(
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPSCPAPEFLGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLGK,
or an equivalent thereof.

### Human IgA1 Constant Region, Uniprot: P01876

(SEQ ID NO: 105(
ASPTSPKVFPLSLCSTQPDGNVVIACLVQGFFPQEPLSVTWSESGQGVTA
RNFPPSQDASGDLYTTSSQLTLPATQCLAGKSVTCHVKHYTNPSQDVTVP
CPVPSTPPTPSPSTPPTPSPSCCHPRLSLHRPALEDLLLGSEANLTCTLT
GLRDASGVTFTWTPSSGKSAVQGPPERDLCGCYSVSSVLPGCAEPWNHGK
TFTCTAAYPESKTPLTATLSKSGNTFRPEVHLLPPPSEELALNELVTLTC
LARGFSPKDVLVRWLOGSQELPREKYLTWASRQEPSQGTTTFAVTSILRV
AAEDWKKGDTFSCMVGHEALPLAFTQKTIDRLAGKPTHVNVSVVMAEVDG
TCY,
or an equivalent thereof.

### Human IgA2 Constant Region, Uniprot: P01877

(SEQ ID NO: 106(
ASPTSPKVFPLSLDSTPQDGNVVVACLVQGFFPQEPLSVTWSESGQNVTA
RNFPPSQDASGDLYTTSSQLTLPATQCPDGKSVTCHVKHYTNPSQDVTVP
CPVPPPPPCCHPRLSLHRPALEDLLLGSEANLTCTLTGLRDASGATFTWT
PSSGKSAVQGPPERDLCGCYSVSSVLPGCAQPWNHGETFTCTAAHPELKT
PLTANITKSGNTFRPEVHLLPPPSEELALNELVTLTCLARGFSPKDVLVR
WLQGSQELPREKYLTWASRQEPSQGTTTFAVTSILRVAAEDWKKGDTFSC
MVGHEALPLAFTQKTIDRMAGKPTHVNVSVVMAEVDGTCY,
or an equivalent thereof.

### Human Ig Kappa Constant Region, Uniprot: P01834 SEQ ID NO: 107

(SEQ ID NO: 107(
TVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKS
FNRGEC,
or an equivalent thereof.

In some aspects of the antibodies provided herein, the antibody contains structural modifications to facilitate rapid binding and cell uptake and/or slow release. In some aspects, the CLDN 18.2 antibody contains a deletion in the CH2 constant heavy chain region of the antibody to facilitate rapid binding and cell uptake and/or slow release. In some aspects, a Fab fragment is used to facilitate rapid binding and cell uptake and/or slow release. In some aspects, a F(ab)′2 fragment is used to facilitate rapid binding and cell uptake and/or slow release.

The antibodies, fragments, and equivalents thereof can be combined with a carrier, e.g., a pharmaceutically acceptable carrier or other agents to provide a formulation for use and/or storage.

Further provided is an isolated polypeptide comprising, or alternatively consisting essentially of, or yet further consisting of, the amino acid sequence of CLDN 18.2 or a fragment thereof, that are useful to generate antibodies that bind to CLDN 18.2, as well as isolated polynucleotides that encode them. In one aspect, the isolated polypeptides or polynucleotides further comprise a label and/or contiguous polypeptide sequences (e.g., keyhole limpet haemocyanin (KLH) carrier protein) or in the case of polynucleotides, polynucleotides encoding the sequence, operatively coupled to polypeptide or polynucleotide. The polypeptides or polynucleotides can be combined with various carriers, e.g., phosphate buffered saline. Further provided are host cells, e.g., prokaryotic or eukaryotic cells, e.g., bacteria, yeast, mammalian (rat, simian, hamster, or human), comprising the isolated polypeptides or polynucleotides. The host cells can be combined with a carrier.

Yet further provided are the isolated nucleic acids encoding the antibodies and fragments thereof as disclosed herein. They can be combined with a vector or appropriate host cell, and/or a suitable carrier for diagnostic or therapeutic use. In one aspect, the nucleic acids are contained with a host cell for recombinant production of polypeptides and proteins. The host cells can be eukaryotic or prokaryotic.

### II. Processes for Preparing Compositions

Antibodies, their manufacture and uses are well known and disclosed in, for example, Harlow, E. and Lane, D., Antibodies: A Laboratory Manual, Cold Spring Harbor Laboratory Press, Cold Spring Harbor, N.Y., 1999. The antibodies may be generated using standard methods known in the art. Examples of antibodies include (but are not limited to) monoclonal, single chain, and functional fragments of antibodies.

Antibodies may be produced in a range of hosts, for example goats, rabbits, rats, mice, humans, and others. They may be immunized by injection with a target antigen or a fragment or oligopeptide thereof which has immunogenic properties, such as an N-terminal or C-terminal fragment of CLDN 18.2 or an isolated polypeptide. Depending on the host species, various adjuvants may be added and used to increase an immunological response. Such adjuvants include, but are not limited to, Freund's, mineral gels such as aluminum hydroxide, and surface active substances such as lysolecithin, pluronic polyols, polyanions, peptides, oil emulsions, keyhole limpet hemocyanin, and dinitrophenol. Among adjuvants used in humans, BCG (Bacille Calmette-Guerin) and Corynebacterium parvum are particularly useful. Thus, this disclosure also provides the isolated polypeptide and an adjuvant.

In certain aspects, the antibodies of the present disclosure are polyclonal, i.e., a mixture of plural types of anti-CLDN 18.2 antibodies having different amino acid sequences. In one aspect, the polyclonal antibody comprises a mixture of plural types of anti-CLDN 18.2 antibodies having different CDRs. As such, a mixture of cells which produce different antibodies is cultured, and an antibody purified from the resulting culture can be used (see WO 2004/061104).

Monoclonal Antibody Production. Monoclonal antibodies to CLDN 18.2 may be prepared using any technique which provides for the production of antibody molecules by continuous cell lines in culture. Such techniques include, but are not limited to, the hybridoma technique (see, e.g., Kohler & Milstein, Nature 256:495-497 (1975)); the trioma technique; the human B-cell hybridoma technique (see, e.g., Kozbor, et al., Immunol. Today 4:72 (1983)) and the EBV hybridoma technique to produce human monoclonal antibodies (see, e.g., Cole, et al., in: MONOCLONAL ANTIBODIES AND CANCER THERAPY, Alan R. Liss, Inc., pp. 77-96 (1985)). Human monoclonal antibodies can be utilized in the practice of the present technology and can be produced by using human hybridomas (see, e.g., Cote, et al., Proc. Natl. Acad. Sci. 80:2026-2030 (1983)) or by transforming human B-cells with Epstein Barr Virus in vitro (see, e.g., Cole, et al., in: MONOCLONAL ANTIBODIES AND CANCER THERAPY, Alan R. Liss, Inc., pp. 77-96 (1985)). For example, a population of nucleic acids that encode regions of antibodies can be isolated. PCR utilizing primers derived from sequences encoding conserved regions of antibodies is used to amplify sequences encoding portions of antibodies from the population and then reconstruct DNAs encoding antibodies or fragments thereof, such as variable domains, from the amplified sequences. Such amplified sequences also can be fused to DNAs encoding other proteins—e.g., a bacteriophage coat, or a bacterial cell surface protein—for expression and display of the fusion polypeptides on phage or bacteria. Amplified sequences can then be expressed and further selected or isolated based, e.g., on the affinity of the expressed antibody or fragment thereof for an antigen or epitope present on the CLDN 18.2 polypeptide. Alternatively, hybridomas expressing anti-CLDN 18.2 monoclonal antibodies can be prepared by immunizing a subject, e.g., with an isolated polypeptide comprising, or alternatively consisting essentially of, or yet further consisting of, the amino acid sequence of CLDN 18.2 or a fragment thereof, and then isolating hybridomas from the subject's spleen using routine methods. See, e.g., Milstein et al., (Galfre and Milstein, Methods Enzymol 73:3-46 (1981)). Screening the hybridomas using standard methods will produce monoclonal antibodies of varying specificity (i.e., for different epitopes) and affinity. A selected monoclonal antibody with the desired properties, e.g., CLDN 18.2 binding, can be (i) used as expressed by the hybridoma, (ii) bound to a molecule such as polyethylene glycol (PEG) to alter its properties, or (iii) a cDNA encoding the monoclonal antibody can be isolated, sequenced and manipulated in various ways. In one aspect, the anti-CLDN 18.2 monoclonal antibody is produced by a hybridoma which includes a B cell obtained from a transgenic non-human animal, e.g., a transgenic mouse, having a genome comprising a human heavy chain transgene and a light chain transgene fused to an immortalized cell. Hybridoma techniques include those known in the art and taught in Harlow et al., Antibodies: A Laboratory Manual Cold Spring Harbor Laboratory, Cold Spring Harbor, N.Y., 349 (1988); Hammerling et al., Monoclonal Antibodies And T-Cell Hybridomas, 563-681 (1981).

Phage Display Technique. As noted above, the antibodies of the present disclosure can be produced through the application of recombinant DNA and phage display technology. For example, anti-CLDN 18.2 antibodies, can be prepared using various phage display methods known in the art. In phage display methods, functional antibody domains are displayed on the surface of a phage particle which carries polynucleotide sequences encoding them. Phage with a desired binding property are selected from a repertoire or combinatorial antibody library (e.g., human or murine) by selecting directly with an antigen, typically an antigen bound or captured to a solid surface or bead. Phage used in these methods are typically filamentous phage including fd and M13 with Fab, Fv or disulfide stabilized Fv antibody domains are recombinantly fused to either the phage gene III or gene VIII protein. In addition, methods can be adapted for the construction of Fab expression libraries (see, e.g., Huse, et al., Science 246:1275-1281, 1989) to allow rapid and effective identification of monoclonal Fab fragments with the desired specificity for a CLDN 18.2 polypeptide, e.g., a polypeptide or derivatives, fragments, analogs or homologs thereof. Other examples of phage display methods that can be used to make the isolated antibodies of the present disclosure include those disclosed in Huston et al., Proc. Natl. Acad. Sci. U.S.A., 85:5879-5883 (1988); Chaudhary et al., Proc. Natl. Acad. Sci. U.S.A., 87:1066-1070 (1990); Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187:9-18 (1997); Burton et al., Advances in Immunology 57:191-280 (1994); PCT/GB91/01134; WO 90/02809; WO 91/10737; WO 92/01047; WO 92/18619; WO 93/11236; WO 95/15982; WO 95/20401; WO 96/06213; WO 92/01047 (Medical Research Council et al.); WO 97/08320 (Morphosys); WO 92/01047 (CAT/MRC); WO 91/17271 (Affymax); and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727 and 5,733,743.

Methods useful for displaying polypeptides on the surface of bacteriophage particles by attaching the polypeptides via disulfide bonds have been described by Lohning, U.S. Pat. No. 6,753,136. As described in the above references, after phage selection, the antibody coding regions from the phage can be isolated and used to generate whole antibodies, including human antibodies, or any other desired antigen binding fragment, and expressed in any desired host including mammalian cells, insect cells, plant cells, yeast, and bacteria. For example, techniques to recombinantly produce Fab, Fab′ and F(ab′)₂ fragments can also be employed using methods known in the art such as those disclosed in WO 92/22324; Mullinax et al., BioTechniques 12:864-869 (1992); Sawai et al., AJRI 34:26-34 (1995); and Better et al., Science 240:1041-1043 (1988).

Generally, hybrid antibodies or hybrid antibody fragments that are cloned into a display vector can be selected against the appropriate antigen in order to identify variants that maintained good binding activity, because the antibody or antibody fragment will be present on the surface of the phage or phagemid particle. See e.g. Barbas III et al., Phage Display, A Laboratory Manual (Cold Spring Harbor Laboratory Press, Cold Spring Harbor, N.Y., 2001). However, other vector formats could be used for this process, such as cloning the antibody fragment library into a lytic phage vector (modified T7 or Lambda Zap systems) for selection and/or screening.

Alternate Methods of Antibody Production. Antibodies may also be produced by inducing in vivo production in the lymphocyte population or by screening recombinant immunoglobulin libraries or panels of highly specific binding reagents (Orlandi et al., PNAS 86:3833-3837 (1989); Winter, G. et al., Nature, 349:293-299 (1991)).

Alternatively, techniques for the production of single chain antibodies may be used. Single chain antibodies (scFvs) comprise a heavy chain variable region and a light chain variable region connected with a linker peptide (typically around 5 to 25 amino acids in length). In the scFᵥ, the variable regions of the heavy chain and the light chain may be derived from the same antibody or different antibodies. scFᵥs may be synthesized using recombinant techniques, for example by expression of a vector encoding the scFᵥ in a host organism such as E. coli. DNA encoding scFᵥ can be obtained by performing amplification using a partial DNA encoding the entire or a desired amino acid sequence of a DNA selected from a DNA encoding the heavy chain or the variable region of the heavy chain of the above-mentioned antibody and a DNA encoding the light chain or the variable region of the light chain thereof as a template, by PCR using a primer pair that defines both ends thereof, and further performing amplification combining a DNA encoding a polypeptide linker portion and a primer pair that defines both ends thereof, so as to ligate both ends of the linker to the heavy chain and the light chain, respectively. An expression vector containing the DNA encoding scFᵥ and a host transformed by the expression vector can be obtained according to conventional methods known in the art.

Antigen binding fragments may also be generated, for example the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed to allow rapid and easy identification of monoclonal Fab fragments with the desired specificity (Huse et al., Science, 256:1275-1281 (1989)).

Antibody Modifications. The antibodies of the present disclosure may be multimerized to increase the affinity for an antigen. The antibody to be multimerized may be one type of antibody or a plurality of antibodies which recognize a plurality of epitopes of the same antigen. As a method of multimerization of the antibody, binding of the IgG CH3 domain to two scFᵥ molecules, binding to streptavidin, introduction of a helix-turn-helix motif and the like can be exemplified.

The antibody compositions disclosed herein may be in the form of a conjugate formed between any of these antibodies and another agent (such as an antibody drug conjugate (ADC) or an immunoconjugate). Non-limiting examples suitable to conjugate or fuse with the antibodies disclosed herein include therapeutic agents, e.g. detectable labels such as radioactive labels, an immunomodulator, a hormone, an enzyme, an oligonucleotide, a photoactive therapeutic or diagnostic agent, a cytotoxic agent, a drug or a toxin, an ultrasound enhancing agent, a non-radioactive label, a combination thereof and other such agents known in the art. In some embodiments, the antibodies may be conjugated to therapeutic agents, prodrugs, peptides, proteins, enzymes, viruses, lipids, biological response modifiers, pharmaceutical agents, and/or polyethylene glycol (PEG).

Bispecific Antibodies. CLDN18.2 is over-expressed in cancer tissues but has restricted off-target positivity. It is contemplated that bi-functional molecules that combine an anti-CLDN18.2 antibody or fragment with another molecule or fragment that has specificity (second specificity) to a cytokine, an immune checkpoint, or a cancer or tumor antigen would have synergistic effect in treatments. Thus, the antibody compositions disclosed herein may be in the form of a bispecific antibody comprising the binding domain of the disclosed antibody and the binding domain with a second specificity. In some embodiments, the second specificity is to a molecule selected from IL-1, CD3, CD16, CD19, CD28, and CD64. Other examples include PD-1, PD-L1, CTLA-4, LAG-3 (also known as CD223), CD28, CD122, 4-1BB (also known as CD137), TIM3, OX-40 or OX40L, CD40 or CD40L, CD47, LIGHT, ICOS/ICOSL, GITR/GITRL, TIGIT, CD27, VISTA, B7H3, B7H4, HEVM or BTLA (also known as CD272), killer-cell immunoglobulin-like receptors (KIRs). An abundance of tumor antigens are known in the art and new tumor antigens can be readily identified by screening. Non-limiting examples of tumor antigens include EGFR, Her2, EpCAM, CD20, CD30, CD33, CD47, CD52, CD133, CD73, CEA, gpA33, Mucins, TAG-72, CIX, PSMA, folate-binding protein, GD2, GD3, GM2, VEGF, VEGFR, Integrin, αVβ3, α5β1, ERBB2, ERBB3, MET, IGF1R, EPHA3, TRAILR1, TRAILR2, RANKL, FAP and Tenascin.

Different formats of bispecific antibodies are also provided. In some embodiments, each of the anti-CLDN18.2 fragment and the second fragment each is independently selected from a Fab fragment, a single-chain variable fragment (scFv), or a single-domain antibody. In some embodiments, the bispecific antibody further includes a Fc fragment.

Bifunctional molecules that include not just antibody or antigen binding fragment are also provided. As a tumor antigen targeting molecule, an antibody or antigen-binding fragment specific to CLDN18.2, such as those described here, can be combined with an immune cytokine or ligand optionally through a peptide linker. The linked immune cytokines or ligands include, but not limited to, IL-1, IL-2, IL-3, IL-4, IL-5, IL-6, IL-7, IL-10, IL-12, IL-13, IL-15, GM-CSF, TNF-alpha, CD40L, OX40L, CD27L, CD30L, 4-1BBL, LIGHT and GITRL. Such bi-functional molecules can lead to specific tumor site local immune modulation.

Antibody Screening. Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between CLDN 18.2, or any fragment or oligopeptide thereof and its specific antibody. A two-site, monoclonal-based immunoassay utilizing monoclonal antibodies specific to two non-interfering CLDN 18.2 epitopes may be used, but a competitive binding assay may also be employed (Maddox et al., J. Exp. Med., 158: 1211-1216 (1983)).

Automated immunohistochemistry (IHC) screening of potential anti-CLDN 18.2 antibodies can be performed using a Ventana Medical Systems, Inc (VMSI) Discovery XT and formalin-fixed, paraffin-embedded human tissue on glass slides. Tissue samples first undergo deparaffinization, antigen retrieval, followed by the addition of the potential anti-CLDN 18.2 antibody and a detection antibody. The detection antibody is visualized using a chromogen detection reagent from VMSI. Stained slides are manually screened under a microscope. Samples having a correct primary antibody staining pattern are selected as potential anti-CLDN 18.2 candidates.

Antibody Purification. The antibodies disclosed herein can be purified to homogeneity. The separation and purification of the antibodies can be performed by employing conventional protein separation and purification methods.

By way of example only, the antibody can be separated and purified by appropriately selecting and combining use of chromatography columns, filters, ultrafiltration, salt precipitation, dialysis, preparative polyacrylamide gel electrophoresis, isoelectric focusing electrophoresis, and the like. Strategies for Protein Purification and Characterization: A Laboratory Course Manual, Daniel R. Marshak et al. eds., Cold Spring Harbor Laboratory Press (1996); Antibodies: A Laboratory Manual. Ed Harlow and David Lane, Cold Spring Harbor Laboratory (1988).

Examples of chromatography include affinity chromatography, ion exchange chromatography, hydrophobic chromatography, gel filtration chromatography, reverse phase chromatography, and adsorption chromatography. In one aspect, chromatography can be performed by employing liquid chromatography such as HPLC or FPLC.

In one aspect, a Protein A column or a Protein G column may be used in affinity chromatography. Other exemplary columns include a Protein A column, Hyper D, POROS, Sepharose F. F. (Pharmacia) and the like.

CAR T-cells are genetically engineered autologous T-cells in which single chain antibody fragments (scFv) or ligands are attached to the T-cell signaling domain capable of facilitating T-cell activation (Maher, J. (2012) ISRN Oncol. 2012:278093; Curran, K. J. et al. (2012) J. Gene Med. 14:405-415; Fedorov, V. D. et al. (2014) Cancer J. 20:160-165; Barrett, D. M. et al. (2014) Annu. Rev. Med. 65:333-347). CARs combine HLA-independent targeting specificity of a monoclonal antibody with the cytolytic activity and homing properties of activated T-cells. These properties enable the recognition of target cells with reduced HLA expression or down-regulated antigen processing pathways, two common methods tumors employ to evade the host immune response (Jakobsen, M. K. et al. (1995) J. Immunother. Emphasis Tumor Immunol. 17:222-228; Lou, Y. et al. (2008) Clin. Cancer Res. 14:1494-1501; Singh, R. et al. (2007) Cancer Res. 67:1887-1892). CAR-modified T-cells have shown great promise in preclinical and clinical settings as novel therapeutics in various diseases including ovarian carcinomas (Chu, C. S. et al. (2008) Expert Rev. Anticancer Ther. 8:243-257; Chekmasova, A. A. et al. (2010) Discov. Med. 9:62-70; Porter, D. L. et al. (2011) NEJM 365:725-733). To date, CAR T-cells generated against a variety of targets. In addition, antibodies to CLDN 18.2 have been disclosed and approved for treatment of certain cancers. Consistent with these principles and discoveries, this disclosure provides the following embodiments.

### III. Methods of Use

General. The antibodies disclosed herein are useful in methods known in the art relating to the localization and/or quantitation of a CLDN 18.2 polypeptide (e.g., for use in measuring levels of the CLDN 18.2 polypeptide within appropriate physiological samples, for use in diagnostic methods, for use in imaging the polypeptide, and the like). The antibodies disclosed herein are useful in isolating a CLDN 18.2 polypeptide by standard techniques, such as affinity chromatography or immunoprecipitation. A CLDN 18.2 antibody disclosed herein can facilitate the purification of natural CLDN 18.2 polypeptides from biological samples, e.g., mammalian sera or cells as well as recombinantly-produced CLDN 18.2 polypeptides expressed in a host system. Moreover, CLDN 18.2 antibody can be used to detect a CLDN 18.2 polypeptide (e.g., in plasma, a cellular lysate or cell supernatant) in order to evaluate the abundance and pattern of expression of the polypeptide. The CLDN 18.2 antibodies disclosed herein can be used diagnostically to monitor CLDN 18.2 levels in tissue as part of a clinical testing procedure, e.g., to determine the efficacy of a given treatment regimen and/or to determine and identify subjects or patient who are most likely to respond to the treatment because the tumors or cancer cells express the antigen. The detection can be facilitated by coupling (i.e., physically linking) the CLDN 18.2 antibodies disclosed herein to a detectable substance.

In another aspect, provided herein is a composition comprising an antibody or antigen binding fragment as disclosed herein bound to a peptide comprising, for example, a human CLDN 18.2 protein or a fragment thereof. In one aspect, the peptide is associated with a cell. For example, the composition may comprise a disaggregated cell sample labeled with an antibody or antibody fragment as disclosed herein, which composition is useful in, for example, affinity chromatography methods for isolating cells or for flow cytometry-based cellular analysis or cell sorting. As another example, the composition may comprise a fixed tissue sample or cell smear labeled with an antibody or antibody fragment as disclosed herein, which composition is useful in, for example, immunohistochemistry or cytology analysis. In another aspect, the antibody or the antibody fragment is bound to a solid support, which is useful in, for example: ELISAs; affinity chromatography or immunoprecipitation methods for isolating CLDN 18.2 proteins or fragments thereof, CLDN 18.2-positive cells, or complexes containing CLDN 18.2 and other cellular components. In another aspect, the peptide is bound to a solid support. For example, the peptide may be bound to the solid support via a secondary antibody specific for the peptide, which is useful in, for example, sandwich ELISAs. As another example, the peptide may be bound to a chromatography column, which is useful in, for example, isolation or purification of antibodies according to the present technology. In another aspect, the peptide is disposed in a solution, such as a lysis solution or a solution containing a sub-cellular fraction of a fractionated cell, which is useful in, for example, ELISAs and affinity chromatography or immunoprecipitation methods of isolating CLDN 18.2 proteins or fragments thereof or complexes containing CLDN 18.2 and other cellular components. In another aspect, the peptide is associated with a matrix, such as, for example, a gel electrophoresis gel or a matrix commonly used for western blotting (such as membranes made of nitrocellulose or polyvinylidene difluoride), which compositions are useful for electrophoretic and/or immunoblotting techniques, such as Western blotting.

Detection of CLDN 18.2 Polypeptide. An exemplary method for detecting the level of CLDN 18.2 polypeptides in a biological sample involves obtaining a biological sample from a subject and contacting the biological sample with a CLDN 18.2 antibody disclosed herein which is capable of detecting the CLDN 18.2 polypeptides.

In one aspect, the CLDN 18.2 antibodies T155S2, T158S2, T224S8, T192S2, and T252S4, or fragments thereof are detectably labeled. The term “labeled”, with regard to the antibody is intended to encompass direct labeling of the antibody by coupling (i.e., physically linking) a detectable substance to the antibody, as well as indirect labeling of the antibody by reactivity with another compound that is directly labeled. Non-limiting examples of indirect labeling include detection of a primary antibody using a fluorescently-labeled secondary antibody and end-labeling of a DNA probe with biotin such that it can be detected with fluorescently-labeled streptavidin.

The detection method of the present disclosure can be used to detect expression levels of CLDN 18.2 polypeptides in a biological sample in vitro as well as in vivo. In vitro techniques for detection of CLDN 18.2 polypeptides include enzyme linked immunosorbent assays (ELISAs), Western blots, flow cytometry, immunoprecipitations, radioimmunoassay, and immunofluorescence (e.g., IHC). Furthermore, in vivo techniques for detection of CLDN 18.2 polypeptides include introducing into a subject a labeled anti-CLDN 18.2 antibody. By way of example only, the antibody can be labeled with a radioactive marker whose presence and location in a subject can be detected by standard imaging techniques. In one aspect, the biological sample contains polypeptide molecules from the test subject.

Immunoassay and Imaging. A CLDN 18.2 antibody disclosed herein can be used to assay CLDN 18.2 polypeptide levels in a biological sample (e.g. human plasma) using antibody-based techniques. For example, protein expression in tissues can be studied with classical immunohistochemical (IHC) staining methods. Jalkanen, M. et al., J. Cell. Biol. 101:976-985 (1985); Jalkanen, M. et al., J. Cell. Biol. 105:3087-3096 (1987). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase, and radioisotopes or other radioactive agents, such as iodine (¹²⁵I, ¹²¹I, ¹³¹I), carbon (¹⁴C), sulfur (³⁵S), tritium (³H), indium (¹¹²In), and technetium (⁹⁹mTc), and fluorescent labels, such as fluorescein and rhodamine, and biotin.

In addition to assaying CLDN 18.2 polypeptide levels in a biological sample, CLDN 18.2 polypeptide levels can also be detected in vivo by imaging. Labels that can be incorporated with anti-CLDN 18.2 antibodies for in vivo imaging of CLDN 18.2 polypeptide levels include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which can be incorporated into the CLDN 18.2 antibody by labeling of nutrients for the relevant scFᵥ clone.

A CLDN 18.2 antibody which has been labeled with an appropriate detectable imaging moiety, such as a radioisotope (e.g., ¹³¹I, ¹¹²In, ⁹⁹mTc), a radio-opaque substance, or a material detectable by nuclear magnetic resonance, is introduced (e.g., parenterally, subcutaneously, or intraperitoneally) into the subject. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ⁹⁹mTc. The labeled CLDN 18.2 antibody will then preferentially accumulate at the location of cells which contain the specific target polypeptide. For example, in vivo tumor imaging is described in S. W. Burchiel et al., Tumor Imaging: The Radiochemical Detection of Cancer 13 (1982).

In some aspects, CLDN 18.2 antibodies containing structural modifications that facilitate rapid binding and cell uptake and/or slow release are useful in in vivo imaging detection methods. In some aspects, the CLDN 18.2 antibody contains a deletion in the CH2 constant heavy chain region of the antibody to facilitate rapid binding and cell uptake and/or slow release. In some aspects, a Fab fragment is used to facilitate rapid binding and cell uptake and/or slow release. In some aspects, a F(ab)′2 fragment is used to facilitate rapid binding and cell uptake and/or slow release.

Diagnostic Uses of CLDN 18.2 antibodies. The CLDN 18.2 antibody compositions disclosed herein are useful in diagnostic and prognostic methods. As such, the present disclosure provides methods for using the antibodies disclosed herein in the diagnosis of CLDN 18.2-related medical conditions in a subject. Antibodies disclosed herein may be selected such that they have a high level of epitope binding specificity and high binding affinity to the CLDN 18.2 polypeptide. In general, the higher the binding affinity of an antibody, the more stringent wash conditions can be performed in an immunoassay to remove nonspecifically bound material without removing the target polypeptide. Accordingly, CLDN 18.2 antibodies of the present technology useful in diagnostic assays usually have binding affinities of at least 10⁻⁶, 10⁻⁷, 10⁻⁸, 10⁻⁹, 10⁻¹⁰, 10⁻¹¹, or 10⁻¹² M. In certain aspects, CLDN 18.2 antibodies used as diagnostic reagents have a sufficient kinetic on-rate to reach equilibrium under standard conditions in at least 12 hours, at least 5 hours, at least 1 hour, or at least 30 minutes.

Some methods of the present technology employ polyclonal preparations of anti-CLDN 18.2 antibodies and polyclonal anti-CLDN 18.2 antibody compositions as diagnostic reagents, and other methods employ monoclonal isolates. In methods employing polyclonal human anti-CLDN 18.2 antibodies prepared in accordance with the methods described above, the preparation typically contains an assortment of CLDN 18.2 antibodies, e.g., antibodies, with different epitope specificities to the target polypeptide. The monoclonal anti-CLDN 18.2 antibodies of the present disclosure are useful for detecting a single antigen in the presence or potential presence of closely related antigens.

The CLDN 18.2 antibodies of the present disclosure can be used as diagnostic reagents for any kind of biological sample. In one aspect, the CLDN 18.2 antibodies disclosed herein are useful as diagnostic reagents for human biological samples. CLDN 18.2 antibodies can be used to detect CLDN 18.2 polypeptides in a variety of standard assay formats. Such formats include immunoprecipitation, Western blotting, ELISA, radioimmunoassay, flow cytometry, IHC and immunometric assays. Sec Harlow & Lane, Antibodies, A Laboratory Manual (Cold Spring Harbor Publications, New York, 1988); U.S. Pat. Nos. 3,791,932; 3,839,153; 3,850,752; 3,879,262; 4,034,074, 3,791,932; 3,817,837; 3,839,153; 3,850,752; 3,850,578; 3,853,987; 3,867,517; 3,879,262; 3,901,654; 3,935,074; 3,984,533; 3,996,345; 4,034,074; and 4,098,876. Biological samples can be obtained from any tissue (including biopsies), cell or body fluid of a subject.

Prognostic Uses of CLDN 18.2 antibodies. The present disclosure also provides for prognostic (or predictive) assays for determining whether a subject is at risk of developing a medical disease or condition associated with increased CLDN 18.2 polypeptide expression or activity (e.g., detection of a precancerous cell). Such assays can be used for prognostic or predictive purpose to thereby prophylactically treat an individual prior to the onset of a medical disease or condition characterized by or associated with CLDN 18.2 polypeptide expression.

Another aspect of the present disclosure provides methods for determining CLDN 18.2 expression in a subject to thereby select appropriate therapeutic or prophylactic compounds for that subject.

Alternatively, the prognostic assays can be utilized to identify a subject having or at risk for developing gastric, pancreatic, esophageal, ovarian, or lung cancer. Thus, the present disclosure provides a method for identifying a disease or condition associated with increased CLDN 18.2 polypeptide expression levels in which a test sample is obtained from a subject and the CLDN 18.2 polypeptide detected, wherein the presence of increased levels of CLDN 18.2 polypeptides compared to a control sample is predictive for a subject having or at risk of developing a disease or condition associated with increased CLDN 18.2 polypeptide expression levels. In some aspects, the disease or condition associated with increased CLDN 18.2 polypeptide expression levels is selected from the group consisting of gastric, pancreatic, esophageal, ovarian, and lung cancer.

In another aspect, the present disclosure provides methods for determining whether a subject can be effectively treated with a compound for a disorder or condition associated with increased CLDN 18.2 polypeptide expression wherein a biological sample is obtained from the subject and the CLDN 18.2 polypeptide is detected using the CLDN 18.2 antibody. The expression level of the CLDN 18.2 polypeptide in the biological sample obtained from the subject is determined and compared with the CLDN 18.2 expression levels found in a biological sample obtained from a subject who is free of the disease. Elevated levels of the CLDN 18.2 polypeptide in the sample obtained from the subject suspected of having the disease or condition compared with the sample obtained from the healthy subject is indicative of the CLDN 18.2-associated disease or condition in the subject being tested.

There are a number of disease states in which the elevated expression level of CLDN 18.2 polypeptides is known to be indicative of whether a subject with the disease is likely to respond to a particular type of therapy or treatment. Thus, the method of detecting a CLDN 18.2 polypeptide in a biological sample can be used as a method of prognosis, e.g., to evaluate the likelihood that the subject will respond to the therapy or treatment. The level of the CLDN 18.2 polypeptide in a suitable tissue or body fluid sample from the subject is determined and compared with a suitable control, e.g., the level in subjects with the same disease but who have responded favorably to the treatment.

In one aspect, the present disclosure provides for methods of monitoring the influence of agents (e.g., drugs, compounds, or small molecules) on the expression of CLDN 18.2 polypeptides. Such assays can be applied in basic drug screening and in clinical trials. For example, the effectiveness of an agent to decrease CLDN 18.2 polypeptide levels can be monitored in clinical trials of subjects exhibiting elevated expression of CLDN 18.2, e.g., patients diagnosed with cancer. An agent that affects the expression of CLDN 18.2 polypeptides can be identified by administering the agent and observing a response. In this way, the expression pattern of the CLDN 18.2 polypeptide can serve as a marker, indicative of the physiological response of the subject to the agent. Accordingly, this response state may be determined before, and at various points during, treatment of the subject with the agent.

Further aspects of the present disclosure relate to methods for determining if a patient is likely to respond or is not likely to CLDN 18.2 CAR therapy. In specific embodiments, this method comprises contacting a tumor sample isolated from the patient with an effective amount of an CLDN 18.2 antibody and detecting the presence of any antibody bound to the tumor sample. In further embodiments, the presence of antibody bound to the tumor sample indicates that the patient is likely to respond to the CLDN 18.2 CAR therapy and the absence of antibody bound to the tumor sample indicates that the patient is not likely to respond to the CLDN 18.2 therapy. In some embodiments, the method comprises the additional step of administering an effective amount of the CLDN 18.2 CAR therapy to a patient that is determined likely to respond to the CLDN 18.2 CAR therapy.

Automated Embodiments. A person of ordinary skill in the art will appreciate that aspects of the methods for using the CLDN 18.2 antibodies disclosed herein can be automated. Particular aspects of CLDN 18.2 staining procedures can be conducted using various automated processes.

### IV. Kits

As set forth herein, the present disclosure provides diagnostic methods for determining the expression level of CLDN 18.2. In one particular aspect, the present disclosure provides kits for performing these methods as well as instructions for carrying out the methods of the present disclosure such as collecting tissue and/or performing the screen, and/or analyzing the results.

The kit comprises, or alternatively consists essentially of, or yet further consists of, a CLDN 18.2 antibody composition (e.g., monoclonal antibodies) disclosed herein, and instructions for use. The kits are useful for detecting the presence of CLDN 18.2 polypeptides in a biological sample e.g., any body fluid including, but not limited to, e.g., sputum, serum, plasma, lymph, cystic fluid, urine, stool, cerebrospinal fluid, acidic fluid or blood and including biopsy samples of body tissue. The test samples may also be a tumor cell, a normal cell adjacent to a tumor, a normal cell corresponding to the tumor tissue type, a blood cell, a peripheral blood lymphocyte, or combinations thereof. The test sample used in the above-described method will vary based on the assay format, nature of the detection method and the tissues, cells or extracts used as the sample to be assayed. Methods for preparing protein extracts or membrane extracts of cells are known in the art and can be readily adapted in order to obtain a sample which is compatible with the system utilized.

In some aspects, the kit can comprise: one or more CLDN 18.2 antibodies capable of binding a CLDN 18.2 polypeptide in a biological sample (e.g., an antibody or antigen-binding fragment thereof having the same antigen-binding specificity of CLDN 18.2 antibody B7H4 5F6, B7H4 #33-14, or B7H4 #36-1); means for determining the amount of the CLDN 18.2 polypeptide in the sample; and means for comparing the amount of the CLDN 18.2 polypeptide in the sample with a standard. One or more of the CLDN 18.2 antibodies may be labeled. The kit components, (e.g., reagents) can be packaged in a suitable container. The kit can further comprise instructions for using the kit to detect the CLDN 18.2 polypeptides. In certain aspects, the kit comprises a first antibody, e.g., attached to a solid support, which binds to a CLDN 18.2 polypeptide; and, optionally; 2) a second, different antibody which binds to either the CLDN 18.2 polypeptide or the first antibody and is conjugated to a detectable label.

The kit can also comprise, e.g., a buffering agent, a preservative or a protein-stabilizing agent. The kit can further comprise components necessary for detecting the detectable-label, e.g., an enzyme or a substrate. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present disclosure may contain a written product on or in the kit container. The written product describes how to use the reagents contained in the kit.

As amenable, these suggested kit components may be packaged in a manner customary for use by those of skill in the art. For example, these suggested kit components may be provided in solution or as a liquid dispersion or the like.

### V. Carriers

The antibodies or CLDN 18.2 CARs also can be bound to many different carriers. Thus, this disclosure also provides compositions containing the antibodies and another substance, active or inert. Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the disclosure. Those skilled in the art will know of other suitable carriers for binding antibodies, or will be able to ascertain such, using routine experimentation.

### Chimeric Antigen Receptors and Uses Thereof

### I. Compositions

The present disclosure provides chimeric antigen receptors (CAR) that bind to CLDN 18.2 comprising, or consisting essentially of, an extracellular and intracellular domain. The extracellular domain comprises a target-specific binding element otherwise referred to as the antigen binding domain. The intracellular domain or cytoplasmic domain comprises a costimulatory signaling region and a zeta chain portion. The CAR may optionally further comprise a spacer domain of up to 300 amino acids, preferably 10 to 100 amino acids, more preferably 25 to 50 amino acids.

Antigen Binding Domain. In certain aspects, the present disclosure provides a CAR that comprises, or alternatively consists essentially thereof, or yet consists of an antigen binding domain specific to CLDN 18.2. In some embodiments, the antigen binding domain comprises, or alternatively consists essentially thereof, or yet consists of the antigen binding domain of an anti-CLDN 18.2 antibody. In further embodiments, the heavy chain variable region and light chain variable region of an anti-CLDN 18.2 antibody comprises, or alternatively consists essentially thereof, or yet consists of the antigen binding domain the anti-CLDN 18.2 antibody. These antibodies and their sequences are disclosed herein. As is apparent to the skilled artisan, the antibody fragments against CLDN 18.2 as disclosed herein can be used to generate a CAR. Thus, the relevant disclosure is incorporated herein.

Transmembrane Domain. The transmembrane domain may be derived either from a natural or from a synthetic source. Where the source is natural, the domain may be derived from any membrane-bound or transmembrane protein. Transmembrane regions of particular use in this invention may be derived from CD8, CD28, CD3, CD45, CD4, CD5, CDS, CD9, CD 16, CD22, CD33, CD37, CD64, CD80, CD86, CD 134, CD137, CD 154, TCR. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

Cytoplasmic Domain. The cytoplasmic domain or intracellular signaling domain of the CAR is responsible for activation of at least one of the traditional effector functions of an immune cell in which a CAR has been placed. The intracellular signaling domain refers to a portion of a protein which transduces the effector function signal and directs the immune cell to perform its specific function. An entire signaling domain or a truncated portion thereof may be used so long as the truncated portion is sufficient to transduce the effector function signal. Cytoplasmic sequences of the TCR and co-receptors as well as derivatives or variants thereof can function as intracellular signaling domains for use in a CAR. Intracellular signaling domains of particular use in this invention may be derived from FcR, TCR, CD3, CDS, CD22, CD79a, CD79b, CD66d. Since signals generated through the TCR are alone insufficient for full activation of a T cell, a secondary or co-stimulatory signal may also be required. Thus, the intracellular region of a co-stimulatory signaling molecule, including but not limited CD27, CD28, 4-IBB (CD 137), OX40, CD30, CD40, PD-1, ICOS, lymphocyte function-associated antigen-1 (LFA-1), CD2, CD7, LIGHT, NKG2C, B7-H3, or a ligand that specifically binds with CD83, to may also be included in the cytoplasmic domain of the CAR.

In some embodiments, the cell activation moiety of the chimeric antigen receptor is a T-cell signaling domain comprising, or alternatively consisting essentially of, or yet further consisting of, one or more proteins or fragments thereof selected from the group consisting of CD8 protein, CD28 protein, 4-1BB protein, and CD3-zeta protein.

In specific embodiments, the CAR comprises, or alternatively consists essentially thereof, or yet consists of an antigen binding domain of an anti-CLDN 18.2 antibody, a CD8α hinge domain, a CD8α transmembrane domain, a costimulatory signaling region, and a CD3 zeta signaling domain. In further embodiments, the costimulatory signaling region comprises either or both a CD28 costimulatory signaling region and a 4-1BB costimulatory signaling region.

In some embodiments, the CAR can further comprise a detectable marker or purification marker.

### II. Process for Preparing CARs

Aspects of the present disclosure relate to an isolated cell comprising a CAR and methods of producing such cells. The cell is a prokaryotic or a eukaryotic cell. In one aspect, the cell is a T-cell, a B cell, a NK cell, a dendritic cell, a myeloid cell, a monocyte, a macrophage, any subsets thereof, or any other immune cell. The eukaryotic cell can be from any preferred species, e.g., an animal cell, a mammalian cell such as a human, a feline or a canine cell. The cells may be derived from patients, donors, or cell lines, such as those available off-the-shelf.

In specific embodiments, the isolated cell comprises, or alternatively consists essentially of, or yet further consists of an exogenous CAR comprising, or alternatively consisting essentially of, or yet further consisting of, an antigen binding domain of a anti-CLDN 18.2 antibody, a CD8α hinge domain, a CD8α transmembrane domain, a CD28 costimulatory signaling region and/or a 4-1BB costimulatory signaling region, and a CD3 zeta signaling domain. In certain embodiments, the isolated cell is a T-cell, e.g., an animal T-cell, a mammalian T-cell, a feline T-cell, a canine T-cell or a human T-cell. In certain embodiments, the isolated cell is an NK-cell, e.g., an animal NK-cell, a mammalian NK-cell, a feline NK-cell, a canine NK-cell or a human NK-cell. In certain embodiments, the isolated cell is a B-cell, e.g., an animal B-cell, a mammalian B-cell, a feline B-cell, a canine B-cell or a human B-cell. It is appreciated that the same or similar embodiments for each species apply with respect to dendritic cells, myeloid cells, monocytes, macrophages, any subsets of these or the T-cells, NK-cells, and B-cells described, and/or any other immune cells.

In certain embodiments, methods of producing CAR expressing cells are disclosed the method comprising, or alternatively consisting essentially of or yet further consisting of transducing a population of isolated cells with a nucleic acid sequence encoding a CAR. In a further aspect, a subpopulation of cells that have been successfully transduced with said nucleic acid sequence is selected. In some embodiments, the isolated cells are T-cells, an animal T-cell, a mammalian T-cell, a feline T-cell, a canine T-cell or a human T-cell, thereby producing CAR T-cells. In certain embodiments, the isolated cell is an NK-cell, e.g., an animal NK-cell, a mammalian NK-cell, a feline NK-cell, a canine NK-cell or a human NK-cell, thereby producing CAR NK-cells. In some embodiments, the isolated cells are B-cells, an animal B-cell, a mammalian B-cell, a feline B-cell, a canine B-cell or a human B-cell, thereby producing CAR B-cells. It is appreciated that the same or similar embodiments for each species apply with respect to dendritic cells, myeloid cells, monocytes, macrophages, any subsets of these or the T-cells, NK-cells, and B-cells described, and/or any other immune cells.

Sources of Isolated Cells. Prior to expansion and genetic modification of the cells disclosed herein, cells may be obtained from a subject—for instance, in embodiments involving autologous therapy—or a commercially available culture.

Cells can be obtained from a number of sources in a subject, including peripheral blood mononuclear cells, bone marrow, lymph node tissue, cord blood, thymus tissue, tissue from a site of infection, ascites, pleural effusion, spleen tissue, and tumors.

Methods of isolating relevant cells are well known in the art and can be readily adapted to the present application; an exemplary method is described in the examples below. Isolation methods for use in relation to this disclosure include, but are not limited to Life Technologies Dynabeads® system; STEMcell Technologies EasySep™, RoboSep™, RosetteSep™, SepMate™; Miltenyi Biotec MACS™ cell separation kits, and other commercially available cell separation and isolation kits. Particular subpopulations of immune cells may be isolated through the use of beads or other binding agents available in such kits specific to unique cell surface markers. For example, MACS™ CD4+ and CD8+ MicroBeads may be used to isolate CD4+ and CD8+ T-cells. Alternate non-limiting examples of cells that may be isolated according to known techniques include bulked T-cells, NK T-cells, and gamma delta T-cells.

Alternatively, cells may be obtained through commercially available cell cultures, including but not limited to, for T-cells, lines BCL2 (AAA) Jurkat (ATCC® CRL-2902™), BCL2 (S70A) Jurkat (ATCC® CRL-2900™), BCL2 (S87A) Jurkat (ATCC® CRL-2901™), BCL2 Jurkat (ATCC® CRL-2899™), Neo Jurkat (ATCC® CRL-2898™); for B cells, lines AHH-1 (ATCC® CRL-8146™), BC-1 (ATCC® CRL-2230™), BC-2 (ATCC® CRL-2231™), BC-3 (ATCC® CRL-2277™), CA46 (ATCC® CRL-1648™), DG-75 [D.G.-75] (ATCC® CRL-2625™), DS-1 (ATCC® CRL-11102™), EB-3 [EB3] (ATCC® CCL-85™), Z-138 (ATCC #CRL-3001), DB (ATCC CRL-2289), Toledo (ATCC CRL-2631), Pfiffer (ATCC CRL-2632), SR (ATCC CRL-2262), JM-1 (ATCC CRL-10421), NFS-5 C-1 (ATCC CRL-1693); NFS-70 C10 (ATCC CRL-1694), NFS-25 C-3 (ATCC CRL-1695), and SUP-B15 (ATCC CRL-1929); and, for NK cells, lines NK-92 (ATCC® CRL-2407™), NK-92 MI (ATCC® CRL-2408™). Further examples include but are not limited to mature T-cell lines, e.g., Deglis, EBT-8, HPB-MLp-W, HUT 78, HUT 102, Karpas 384, Ki 225, My-La, Se-Ax, SKW-3, SMZ-1 and T34; immature T-cell lines, e.g., ALL-SIL, Be13, CCRF-CEM, CML-T1, DND-41, DU.528, EU-9, HD-Mar, HPB-ALL, H-SB2, HT-1, JK-T1, Jurkat, Karpas 45, KE-37, KOPT-K1, K-T1, L-KAW, Loucy, MAT, MOLT-1, MOLT 3, MOLT-4, MOLT 13, MOLT-16, MT-1, MT-ALL, P12/Ichikawa, Peer, PER0117, PER-255, PF-382, PFI-285, RPMI-8402, ST-4, SUP-T1 to T14, TALL-1, TALL-101, TALL-103/2, TALL-104, TALL-105, TALL-106, TALL-107, TALL-197, TK-6, TLBR-1, -2, -3, and -4, CCRF-HSB-2 (CCL-120.1), J.RT3-T3.5 (ATCC TIB-153), J45.01 (ATCC CRL-1990), J.CaM1.6 (ATCC CRL-2063), RS4; 11 (ATCC CRL-1873), CCRF-CEM (ATCC CRM-CCL-119); cutaneous T-cell lymphoma lines, e.g., HuT78 (ATCC CRM-TIB-161), MJ [G11] (ATCC CRL-8294), HuT102 (ATCC TIB-162); B-cell lines derived from anaplastic and large cell lymphomas, e.g., DEL, DL-40, FE-PD, JB6, Karpas 299, Ki-JK, Mac-2A Ply1, SR-786, SU-DHL-1, -2, -4, -5, -6, -7, -8, -9, -10, and -16, DOHH-2, NU-DHL-1, U-937, Granda 519, USC-DHL-1, RL; Hodgkin's lymphomas, e.g., DEV, HD-70, HDLM-2, HD-MyZ, HKB-1, KM-H2, L 428, L 540, L1236, SBH-1, SUP-HD1, and SU/RH-HD-1; and NK lines such as HANK1, KHYG-1, NKL, NK-YS, NOI-90, and YT. Null leukemia cell lines, including but not limited to REH, NALL-1, KM-3, L92-221, are a another commercially available source of immune cells, as are cell lines derived from other leukemias and lymphomas, such as K562 erythroleukemia, THP-1 monocytic leukemia, U937 lymphoma, HEL erythroleukemia, HL60 leukemia, HMC-1 leukemia, KG-1 leukemia, U266 myeloma. Non-limiting exemplary sources for such commercially available cell lines include the American Type Culture Collection, or ATCC, (http://www.atcc.org/) and the German Collection of Microorganisms and Cell Cultures (https://www.dsmz.de/).

In some embodiments, T-cells expressing the disclosed CARs may be further modified to reduce or eliminate expression of endogenous TCRs. Reduction or elimination of endogenous TCRs can reduce off-target effects and increase the effectiveness of the T cells. T cells stably lacking expression of a functional TCR may be produced using a variety of approaches. T cells internalize, sort, and degrade the entire T cell receptor as a complex, with a half-life of about 10 hours in resting T cells and 3 hours in stimulated T cells (von Essen, M. et al. 2004. J. Immunol. 173:384-393). Proper functioning of the TCR complex requires the proper stoichiometric ratio of the proteins that compose the TCR complex. TCR function also requires two functioning TCR zeta proteins with ITAM motifs. The activation of the TCR upon engagement of its MHC-peptide ligand requires the engagement of several TCRs on the same T cell, which all must signal properly. Thus, if a TCR complex is destabilized with proteins that do not associate properly or cannot signal optimally, the T cell will not become activated sufficiently to begin a cellular response.

Accordingly, in some embodiments, TCR expression may eliminated using RNA interference (e.g., shRNA, siRNA, miRNA, etc.), CRISPR, or other methods that target the nucleic acids encoding specific TCRs (e.g., TCR-α and TCR-β) and/or CD3 chains in primary T cells. By blocking expression of one or more of these proteins, the T cell will no longer produce one or more of the key components of the TCR complex, thereby destabilizing the TCR complex and preventing cell surface expression of a functional TCR. Even though some TCR complexes can be recycled to the cell surface when RNA interference is used, the RNA (e.g., shRNA, siRNA, miRNA, etc.) will prevent new production of TCR proteins resulting in degradation and removal of the entire TCR complex, resulting in the production of a T cell having a stable deficiency in functional TCR expression.

Expression of inhibitory RNAs (e.g., shRNA, siRNA, miRNA, etc.) in primary T cells can be achieved using any conventional expression system, e.g., a lentiviral expression system. Although lentiviruses are useful for targeting resting primary T cells, not all T cells will express the shRNAs. Some of these T cells may not express sufficient amounts of the RNAs to allow enough inhibition of TCR expression to alter the functional activity of the T cell. Thus, T cells that retain moderate to high TCR expression after viral transduction can be removed, e.g., by cell sorting or separation techniques, so that the remaining T cells are deficient in cell surface TCR or CD3, enabling the expansion of an isolated population of T cells deficient in expression of functional TCR or CD3.

Expression of CRISPR in primary T cells can be achieved using conventional CRISPR/Cas systems and guide RNAs specific to the target TCRs. Suitable expression systems, e.g. lentiviral or adenoviral expression systems are known in the art. Similar to the delivery of inhibitor RNAs, the CRISPR system can be use to specifically target resting primary T cells or other suitable immune cells for CAR cell therapy. Further, to the extent that CRISPR editing is unsuccessful, cells can be selected for success according to the methods disclosed above. For example, as noted above, T cells that retain moderate to high TCR expression after viral transduction can be removed, e.g., by cell sorting or separation techniques, so that the remaining T cells are deficient in cell surface TCR or CD3, enabling the expansion of an isolated population of T cells deficient in expression of functional TCR or CD3. It is further appreciated that a CRISPR editing construct may be useful in both knocking out the endogenous TCR and knocking in the CAR constructs disclosed herein. Accordingly, it is appreciated that a CRISPR system can be designed for to accomplish one or both of these purposes.

Vectors. CAR cells may be prepared using vectors. Aspects of the present disclosure relate to an isolated nucleic acid sequence encoding (i) a CAR or (ii) a polynucleotide encoding an immunoregulatory molecule and vectors comprising, or alternatively consisting essentially of, or yet further consisting of, an either one or both of these nucleic acids and/or complements and/or equivalents of each thereof.

In some embodiments, the isolated nucleic acid sequence encodes for a CAR comprising, or alternatively consisting essentially of, or yet further consisting of an antigen binding domain of a cancer or tumor targeting antibody, a CD8α hinge domain, a CD8α transmembrane domain, a CD28 costimulatory signaling region and/or a 4-1BB costimulatory signaling region, and a CD3 zeta signaling domain. In specific embodiments, the isolated nucleic acid sequence comprises, or alternatively consisting essentially thereof, or yet further consisting of, sequences encoding (a) an antigen binding domain of an anti-CLDN 18.2 antibody followed by (b) a CD8α hinge domain, (c) a CD8α transmembrane domain followed by (d) a CD28 costimulatory signaling region and/or a 4-1BB costimulatory signaling region followed by (e) a CD3 zeta signaling domain.

In some embodiments, the isolated nucleic acid sequence encodes for a CAR and comprises, or alternatively consists essentially of, or yet further consists of, a Kozak consensus sequence upstream of the sequence encoding the antigen binding domain of the anti-CLDN 18.2 antibody.

In some embodiments, the isolated nucleic acid comprises a detectable label and/or a polynucleotide conferring antibiotic resistance. In one aspect, the label or polynucleotide are useful to select cells successfully transduced with the isolated nucleic acids.

In some embodiments, the isolated nucleic acid sequence is comprised within a vector. In certain embodiments, the vector is a plasmid. In other embodiments, the vector is a viral vector. Non-limiting examples of such include without limitation a retroviral vector, a lentiviral vector, an adenoviral vector, and an adeno-associated viral vector. In specific embodiments, the vector is a lentiviral vector.

The preparation of exemplary vectors and the generation of CAR expressing cells using said vectors is discussed in detail in the examples below. In summary, the expression of natural or synthetic nucleic acids encoding CARs or immunoregulatory molecules is typically achieved by operably linking a nucleic acid encoding the CAR polypeptide or portions thereof to a promoter, and incorporating the construct into an expression vector. A similar method may be used to construct the isolated nucleic acid sequence comprising a polynucleotide encoding an immunoregulatory molecule. The vectors can be suitable for replication and integration eukaryotes. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York).

In one aspect, the term “vector” intends a recombinant vector that retains the ability to infect and transduce non-dividing and/or slowly-dividing cells and integrate into the target cell's genome. In several aspects, the vector is derived from or based on a wild-type virus. In further aspects, the vector is derived from or based on a wild-type lentivirus. Examples of such include without limitation, human immunodeficiency virus (HIV), equine infectious anemia virus (EIAV), simian immunodeficiency virus (SIV) and feline immunodeficiency virus (FIV). Alternatively, it is contemplated that other retrovirus can be used as a basis for a vector backbone such murine leukemia virus (MLV). It will be evident that a viral vector according to the disclosure need not be confined to the components of a particular virus. The viral vector may comprise components derived from two or more different viruses, and may also comprise synthetic components. Vector components can be manipulated to obtain desired characteristics such as target cell specificity.

The recombinant vectors of this disclosure are derived from primates and non-primates. Examples of primate lentiviruses include the human immunodeficiency virus (HIV), the causative agent of human acquired immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anemia virus (EIAV) and the more recently described feline immunodeficiency virus (FIV) and bovine immunodeficiency virus (BIV). Prior art recombinant lentiviral vectors are known in the art, e.g., see U.S. Pat. Nos. 6,924,123; 7,056,699; 7,419,829 and 7,442,551, incorporated herein by reference.

U.S. Pat. No. 6,924,123 discloses that certain retroviral sequence facilitate integration into the target cell genome. This patent teaches that each retroviral genome comprises genes called gag, pol and env which code for virion proteins and enzymes. These genes are flanked at both ends by regions called long terminal repeats (LTRs). The LTRs are responsible for proviral integration, and transcription. They also serve as enhancer-promoter sequences. In other words, the LTRs can control the expression of the viral genes. Encapsidation of the retroviral RNAs occurs by virtue of a psi sequence located at the 5′ end of the viral genome. The LTRs themselves are identical sequences that can be divided into three elements, which are called U3, R and U5. U3 is derived from the sequence unique to the 3′ end of the RNA. R is derived from a sequence repeated at both ends of the RNA, and U5 is derived from the sequence unique to the 5′ end of the RNA. The sizes of the three elements can vary considerably among different retroviruses. For the viral genome- and the site of poly (A) addition (termination) is at the boundary between R and U5 in the right hand side LTR. U3 contains most of the transcriptional control elements of the provirus, which include the promoter and multiple enhancer sequences responsive to cellular and in some cases, viral transcriptional activator proteins.

With regard to the structural genes gag, pol and env themselves, gag encodes the internal structural protein of the virus. Gag protein is proteolytically processed into the mature proteins MA (matrix), CA (capsid) and NC (nucleocapsid). The pol gene encodes the reverse transcriptase (RT), which contains DNA polymerase, associated RNase H and integrase (IN), which mediate replication of the genome.

For the production of viral vector particles, the vector RNA genome is expressed from a DNA construct encoding it, in a host cell. The components of the particles not encoded by the vector genome are provided in trans by additional nucleic acid sequences (the “packaging system”, which usually includes either or both of the gag/pol and env genes) expressed in the host cell. The set of sequences required for the production of the viral vector particles may be introduced into the host cell by transient transfection, or they may be integrated into the host cell genome, or they may be provided in a mixture of ways. The techniques involved are known to those skilled in the art.

Retroviral vectors for use in this disclosure include, but are not limited to Invitrogen's pLenti series versions 4, 6, and 6.2 “ViraPower” system. Manufactured by Lentigen Corp.; pHIV-7-GFP, lab generated and used by the City of Hope Research Institute; “Lenti-X” lentiviral vector, pLVX, manufactured by Clontech; pLKO.1-puro, manufactured by Sigma-Aldrich; pLemiR, manufactured by Open Biosystems; and pLV, lab generated and used by Charité Medical School, Institute of Virology (CBF), Berlin, Germany.

Further methods of introducing exogenous nucleic acids into the art are known and include but are not limited to gene delivery using one or more of RNA electroporation, nanotechnology, sleeping beauty vectors, retroviruses, and/or adenoviruses.

Regardless of the method used to introduce exogenous nucleic acids into a host cell or otherwise expose a cell to the inhibitor of the present disclosure, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the disclosure.

Packaging vector and cell lines. The isolated nucleic acids can be packaged into a retroviral packaging system by using a packaging vector and cell lines. The packaging vector includes, but is not limited to retroviral vector, lentiviral vector, adenoviral vector, and adeno-associated viral vector. The packaging vector contains elements and sequences that facilitate the delivery of genetic materials into cells. For example, the retroviral constructs are packaging vectors comprising at least one retroviral helper DNA sequence derived from a replication-incompetent retroviral genome encoding in trans all virion proteins required to package a replication incompetent retroviral vector, and for producing virion proteins capable of packaging the replication-incompetent retroviral vector at high titer, without the production of replication-competent helper virus. The retroviral DNA sequence lacks the region encoding the native enhancer and/or promoter of the viral 5′ LTR of the virus, and lacks both the psi function sequence responsible for packaging helper genome and the 3′ LTR, but encodes a foreign polyadenylation site, for example the SV40 polyadenylation site, and a foreign enhancer and/or promoter which directs efficient transcription in a cell type where virus production is desired. The retrovirus is a leukemia virus such as a Moloney Murine Leukemia Virus (MMLV), the Human Immunodeficiency Virus (HIV), or the Gibbon Ape Leukemia virus (GALV). The foreign enhancer and promoter may be the human cytomegalovirus (HCMV) immediate early (IE) enhancer and promoter, the enhancer and promoter (U3 region) of the Moloney Murine Sarcoma Virus (MMSV), the U3 region of Rous Sarcoma Virus (RSV), the U3 region of Spleen Focus Forming Virus (SFFV), or the HCMV IE enhancer joined to the native Moloney Murine Leukemia Virus (MMLV) promoter. The retroviral packaging vector may consist of two retroviral helper DNA sequences encoded by plasmid based expression vectors, for example where a first helper sequence contains a cDNA encoding the gag and pol proteins of ecotropic MMLV or GALV and a second helper sequence contains a cDNA encoding the env protein. The Env gene, which determines the host range, may be derived from the genes encoding xenotropic, amphotropic, ecotropic, polytropic (mink focus forming) or 10A1 murine leukemia virus env proteins, or the Gibbon Ape Leukemia Virus (GALV env protein, the Human Immunodeficiency Virus env (gp160) protein, the Vesicular Stomatitis Virus (VSV) G protein, the Human T cell leukemia (HTLV) type I and II env gene products, chimeric envelope gene derived from combinations of one or more of the aforementioned env genes or chimeric envelope genes encoding the cytoplasmic and transmembrane of the aforementioned env gene products and a monoclonal antibody directed against a specific surface molecule on a desired target cell.

In the packaging process, the packaging vectors and retroviral vectors are transiently cotransfected into a first population of mammalian cells that are capable of producing virus, such as human embryonic kidney cells, for example 293 cells (ATCC No. CRL1573, ATCC, Rockville, Md.) to produce high titer recombinant retrovirus-containing supernatants. In another method of the disclosure this transiently transfected first population of cells is then cocultivated with mammalian target cells, for example human lymphocytes, to transduce the target cells with the foreign gene at high efficiencies. In yet another method of the invention the supernatants from the above described transiently transfected first population of cells are incubated with mammalian target cells, for example human lymphocytes or hematopoietic stem cells, to transduce the target cells with the foreign gene at high efficiencies.

In another aspect, the packaging vectors are stably expressed in a first population of mammalian cells that are capable of producing virus, such as human embryonic kidney cells, for example 293 cells. Retroviral or lentiviral vectors are introduced into cells by either cotransfection with a selectable marker or infection with pseudotyped virus. In both cases, the vectors integrate. Alternatively, vectors can be introduced in an episomally maintained plasmid. High titer recombinant retrovirus-containing supernatants are produced.

Activation and Expansion of CAR Cells. Whether prior to or after genetic modification of the cells to express a desirable CAR, the cells can be activated and expanded using generally known methods such as those described in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041 and references such as Lapateva et al. (2014) Crit Rev Oncog 19 (1-2): 121-32; Tam et al. (2003) Cytotherapy 5 (3): 259-72; Garcia-Marquez et al. (2014) Cytotherapy 16 (11): 1537-44. Stimulation with the CLDN 18.2 antigen ex vivo can activate and expand the selected CAR expressing cell subpopulation. Alternatively, the cells may be activated in vivo by interaction with CLDN 18.2 antigen.

In the case of certain immune cells, additional cell populations, soluble ligands and/or cytokines, or stimulating agents may be required to activate and expand cells. The relevant reagents are well known in the art and are selected according to known immunological principles. For instance, soluble CD-40 ligand may be helpful in activating and expanding certain B-cell populations; similarly, irradiated feeder cells may be used in the procedure for activation and expansion of NK cells.

Methods of activating relevant cells are well known in the art and can be readily adapted to the present application; an exemplary method is described in the examples below. Isolation methods for use in relation to this disclosure include, but are not limited to Life Technologies Dynabeads® system activation and expansion kits; BD Biosciences Phosflow™ activation kits, Miltenyi Biotec MACS™ activation/expansion kits, and other commercially available cell kits specific to activation moieties of the relevant cell. Particular subpopulations of immune cells may be activated or expanded through the use of beads or other agents available in such kits. For example, α-CD3/α-CD28 Dynabeads® may be used to activate and expand a population of isolated T-cells.

As disclosed above, chimeric antigen receptors comprise an antigen recognition moiety and a cell activation moiety. Aspects of the present disclosure related to a chimeric antigen receptor (CAR) comprising an antigen binding domain specific to CLDN 18.2.

In one embodiment, the CAR of the present disclosure is characterized in that it comprises, or alternatively consists essentially of, or yet further consists an antigen binding domain of an anti-luteinizing hormone receptor (“CLDN 18.2”) antibody, a CD8α hinge domain; a CD8α transmembrane domain; a CD28 and/or a 4-1BB costimulatory signaling region; and a CD3 zeta signaling domain.

In another embodiment, the antigen binding domain of the anti-CLDN 18.2 antibody comprises an anti-CLDN 18.2 heavy chain variable region and an anti-CLDN 18.2 light chain variable region. As apparent to the skilled artisan, antibodies having the detailed elements are also within the scope of this disclosure.

In some embodiments, the isolated nucleic acid further comprises, or alternatively consists essentially of, or yet further consists of a Kozak consensus sequence located upstream of the antigen binding domain of the anti-CLDN 18.2 antibody.

In some aspect of the disclosure, the isolated nucleic sequence further comprises, or alternatively consists essentially of, or yet further consists of polynucleotide encoding an antibiotic resistance marker.

In another embodiment, the disclosure provides a vector comprising, or alternatively consisting essentially of, or yet further consisting of the isolated nucleic acid sequence encoding the anti-CLDN 18.2 CAR polypeptide as disclosed above or a complement thereof. In a further aspect, the polynucleotide can be incorporated into a vector. Non-limiting examples of such include a retroviral vector, a plasmid, a lentiviral vector, an adenoviral vector, and an adeno-associated viral vector.

In one embodiment, the disclosure is an isolated cell comprising, or alternatively consisting essentially of, or yet further consisting of an exogenously added polynucleotide encoding the anti-CLDN 18.2 CAR as described above alone or in combination with a vector as described above; and/or the exogenously added the anti-CLDN 18.2 CAR; as describe above.

In one aspect of the disclosure, the isolated cell can be a T-cell or a natural killer (NK) cell.

### III. Methods of Use

Therapeutic Application. The CAR T-cells of the present invention may be used to treat tumors and cancers. The CAR-T cells of the present invention may be administered either alone or in combination with diluents, known anti-cancer therapeutics, and/or with other components such as cytokines or other cell populations that are immunostimulatory.

Method aspects of the present disclosure relate to methods for inhibiting the growth of a tumor in a subject in need thereof and/or for treating a cancer patient in need thereof. In some embodiments, the tumor is a solid tumor. In some embodiments, the tumors/cancer is gastric, pancreatic, esophageal, ovarian, or lung tumors/cancer. In some embodiments, the tumor or cancer expresses or overexpresses CLDN 18.2. In certain embodiments, these methods comprise, or alternatively consist essentially of, or yet further consist of, administering to the subject or patient an effective amount of the isolated cell. In further embodiments, this isolated cell comprises a CLDN 18.2 CAR. In still further embodiments, the isolated cell is a T-cell or an NK cell. In some embodiments, the isolated cell is autologous to the subject or patient being treated. In a further aspect, the tumor expresses CLDN 18.2 antigen and the subject has been selected for the therapy by a diagnostic, such as the one described herein.

The CAR cells as disclosed herein may be administered either alone or in combination with diluents, known anti-cancer therapeutics, and/or with other components such as cytokines or other cell populations that are immunostimulatory. They may be first line, second line, third line, fourth line, or further therapy. The can be combined with other therapies. Non-limiting examples of such include chemotherapies or biologics. Appropriate treatment regimes will be determined by the treating physician or veterinarian.

Pharmaceutical compositions comprising the CLDN 18.2 CAR of the present invention may be administered in a manner appropriate to the disease to be treated or prevented. The quantity and frequency of administration will be determined by such factors as the condition of the patient, and the type and severity of the patient's disease, although appropriate dosages may be determined by clinical trials.

In another aspect, the disclosure provides a method of inhibiting the growth of a solid tumor in a subject in need thereof, comprising administering to the subject an effective amount of the isolated cell of anti-CLDN 18.2 CAR T-cell or NK-cell. In one embodiment, the isolated T-cells or NK-cells are autologous to the subject being treated. In another embodiment, the tumor is a gastric, colon, colorectal, pancreatic, esophageal, ovarian, or lung tumor. The method of inhibiting the growth of a tumor can be applied to a subject including but not limited to human, dog, cat, horse, and other species.

In another aspect, the disclosure provides a method of treating a cancer patient in need thereof, comprising, or alternatively consisting essentially of, or yet further consisting of administering to the subject an effective amount of the isolated cell of anti-CLDN 18.2 T-cell or NK-cell. The isolated T-cells or NK-cells can be autologous to the subject being treated. The tumor is gastric, pancreatic, esophageal, ovarian, or lung cancer. In one embodiment, the subject treated for cancer is a human patient.

In one embodiment, the disclosure provides a method for determining if a patient is likely to respond or is not likely to anti-CLDN 18.2 CAR therapy, comprising, or alternatively consisting essentially of, or yet further consisting of contacting a tumor sample isolated from the patient with an effective amount of an anti-CLDN 18.2 antibody and detecting the presence of any antibody bound to the tumor sample, wherein the presence of antibody bound to the tumor sample indicates that the patient is likely to respond to the anti-CLDN 18.2 CAR therapy and the absence of antibody bound to the tumor sample indicates that the patient is not likely to respond to the anti-CAR therapy. In another embodiment, the method further comprises administering an effective amount of the anti-CLDN 18.2 CAR therapy to the patient that is determined likely to respond to the anti-CLDN 18.2 CAR therapy. In this method, the patient can suffer from gastric, pancreatic, esophageal, ovarian, or lung cancer.

The present disclosure also provides diagnostic methods for determining the expression level of CLDN 18.2. In one particular aspect, the present disclosure provides kits for performing these methods as well as instructions for carrying out the methods of the present disclosure such as collecting tissue and/or performing the screen, and/or analyzing the results.

In one embodiment, the disclosure provides a method of detecting CLDN 18.2 in a biological sample comprising, or alternatively consisting essentially of, or yet further consisting of contacting the sample with an anti-CLDN 18.2 antibody or an antigen binding fragment capable of binding a peptide comprising SEQ ID NOs: 42 and 43, and detecting a complex formed by the binding of the antibody or antigen binding fragment to CLDN 18.2. In one aspect, the sample comprises a cell sample or a tissue sample.

In one aspect, the sample is obtained from a subject that is diagnosed as having, suspected as having, or at risk of having cancer.

In another aspect, the cancer is gastric, pancreatic, esophageal, ovarian, or lung cancer. In a further aspect, the cancer cell or tumor cell expresses or overexpresses CLDN 18.2.

In one aspect, the detection comprises, or alternatively consists essentially of, or yet further consists of one or more of immunohistochemistry (IHC), Western blotting, Flow cytometry or ELISA.

In one aspect, the method comprises, or alternatively consists essentially of, or yet further consists of isolating the biological sample from the subject.

In one aspect, the subject is a mammal.

In another aspect, the mammal is selected from the group of: a human, an animal, a non-human primate, a dog, cat, a sheep, a mouse, a horse, or a cow.

In one embodiment, the disclosure provides a method of detecting a pathological cell in a sample isolated from a subject, comprising, or alternatively consisting essentially of, or yet further consisting of (a) detecting the level of CLDN 18.2 in a biological sample from the subject by detecting a complex formed by an anti-CLDN 18.2 antibody or an antigen binding fragment capable of binding a peptide comprising CLDN 18.2 and (b) comparing the levels of CLDN 18.2 observed in step (a) with the levels of CLDN 18.2 observed in a control biological sample; wherein the pathological cell is detected when the level of CLDN 18.2 is elevated compared to that observed in the control biological sample.

In one aspect, the biological sample of the subject comprises, or alternatively consists essentially of, or yet further consists of one or more of a sample isolated from gastric, pancreatic, esophageal, ovarian, or lung tissue.

In another aspect, the detection comprises, or alternatively consists essentially of, or yet further consists of one or more of immunohistochemistry (IHC), Western Blotting, Flow cytometry or ELISA.

In one aspect, the method comprises, or alternatively consists essentially of, or yet further consists of isolating the biological sample from the subject.

In one aspect, the subject is a mammal.

In another aspect, the mammal is selected from the group of: a human, an animal, a non-human primate, a dog, cat, a sheep, a mouse, a horse, or a cow.

### IV. Carriers

Additional aspects of the invention relate to compositions comprising a carrier and one or more of the products—e.g., an isolated cell comprising a CLDN 18.2 CAR, an isolated nucleic acid, a vector, an isolated cell of any anti-CLDN 18.2 antibody or CAR cell, an anti-CLDN 18.2—described in the embodiments disclosed herein.

Briefly, pharmaceutical compositions of the present invention including but not limited to any one of the claimed compositions may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure may be formulated for oral, intravenous, topical, enteral, and/or parenteral administration. In certain embodiments, the compositions of the present disclosure are formulated for intravenous administration.

Briefly, pharmaceutical compositions of the present invention including but not limited to any one of the claimed compositions may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.

Pharmaceutical compositions of the present invention may be administered in a manner appropriate to the disease to be treated or prevented. The quantity and frequency of administration will be determined by such factors as the condition of the patient, and the type and severity of the patient's disease, although appropriate dosages may be determined by clinical trials.

The following examples are provided to illustrate, and not limit the disclosure.

### EXAMPLES

### Example 1: Generation of Mouse Monoclonal Antibodies Against Human CLDN18.2

Anti-human CLDN18.2 mouse monoclonal antibodies were generated using the hybridoma technology.

Antigens: a) cDNA encoding human CLDN18.2EC1 (extra-cellular domain 1) human Fc protein; b) human CLDN18.2EC1 (extra-cellular domain 1) human Fc protein; c) CHO cells expressing full length human CLDN18.2.

Immunization: To generate mouse monoclonal antibodies to human CLDN18.2, 4-6 week female Swiss Webster mice were first immunized with cDNAs encoding human CLDN18.2EC1 human Fc protein at 40 ug/mouse. Day 21 and 35 post first immunization, the immunized mice were boosted with 30 ugs of human CLDN18.2EC1 human Fc protein each mouse. The immunized mice were further boosted with CHO cells expressing full length human CLDN18.2. To select mice producing antibodies that bound to human CLDN18.2, sera from immunized mice were tested by ELISA and FACS analysis.

For ELISA assays, microtiter plates were coated with human CLDN18.2 EC1 human Fc protein at 1 μg/ml in PBS, 100 μl/well at 4° C. overnight and blocked with 100 μl/well of 2% BSA for 1 hour at RT. Dilutions of serum from immunized mice were added to each well and incubated for 1-2 hours at RT. The plates were washed with PBS/Tween and then incubate with anti-mouse IgG antibody conjugated with Horse Radish Peroxidase (HRP) for 1 hour at RT. After washing, the plates were developed with TMB peroxidase substrate and analyzed by spectrophotometer at OD 450 nm.

For FACS based titer analysis, 96 well microtiter plates were seeded with human CLDN18.2/CHO cells or CLDN18.1/CHO cells at 200,000 cells/well. Dilutions of serum from immunized mice were added to each well. The cells were mixed well and incubated at 4° C. for 15 min. The cells were then washed with PBS three times, followed by incubation with PE-labeled anti-mouse Fc gamma specific antibody (Jackson Immuno-Research) at 4° C. for 15 min. The cells were washed again with PBS three times and analyzed using an FACS Caliber instrument (Becton-Dickinson). Mice with sufficient specific titers to CLDN18.2 were boosted with 30 μg human CLDN18.2EC1 huFc protein 3-4 days before fusions.

Hybridoma fusions were done using BTX2001 electro-cell manipulator (Harvard Apparatus) and the standard protocol for hybridoma electro-fusion. Total mouse lymphocytes isolated from both lymph nodes and the spleens were fused with SP2/0 cells (ATCC) and hybridoma were selected using media containing Azaserine (Sigma). Hybridoma supernatants were screened using both the ELISA and FACS assays. Positive wells were further subcloned using the limited dilution method.

Hybridoma clones T13S2, T88S1, T138S2, T155S2, T158S2, T192S2, T224S8, T252S4 and LN27S4 were selected for hybridoma sequencing. Briefly, RNAs were extracted from hybridoma cells using Trizol reagents (Invitrogen) and cDNAs were prepared using the PrimeScript™ Reverse Transcriptase). Antibody heavy and light chain V regions were PCR amplified using the cDNAs and the mouse Ig-Primer Set (Novagen, TB326 Rev.B 0503) and sequences were obtained using the standard DNA sequencing techniques.

The amino acid and polynucleotide sequences of the variable regions of the antibodies are provided in Table 1 and Table 2 below.

TABLE 1
CDR combinations for anti- CLDN18.2 antibodies

| Clone | | CDRL combinations | | | CDRH combinations | | |
|---|---|---|---|---|---|---|---|
| Name | Comb # | CDRL1 | CDRL2 | CDRL3 | CDRH1 | CDRH2 | CDRH3 |
| T155S2 | 1 | QNLLNSSNQKNY | FAS | QQHYSTPLT | GFTFSSYA | ISGGGST | GGNIYDGYPYYFDY |
| | SEQ ID | 1 | 2 | 3 | 4 | 5 | 6 |
| | NO : | | | | | | |
| T158S2 | 2 | ESVDNYGFSF | RAS | QQSNOGPLT | GFTFSDYY | ISDGGGDT | ARHRGSLDY |
| | SEQ ID | 7 | 8 | 9 | 10 | 11 | 12 |
| | NO: | | | | | | |
| T224S8 | 3 | QSLVHSNGNTY | KVS | SQNTHVPRT | GFNIKDYF | IDPENGDT | NVLGYGNYGHFYYAMDY |
| | SEQ ID | 13 | 14 | 15 | 16 | 17 | 18 |
| | NO: | | | | | | |
| T192S2 | 4 | ESVDNYGFSF | RAS | QQSNKVPLT | GFTFSDYY | ISNGGGST | ARHRGSLDE |
| | SEQ ID | 19 | 20 | 21 | 22 | 23 | 24 |
| | NO: | | | | | | |
| T252S4 | 5 | ONLINSGNOKNY | WAS | QNDYIYPLP | GYSFTGYN | INPYNGGT | ARMGLGNAMDY |
| | SEQ ID | 25 | 26 | 27 | 28 | 29 | 30 |
| | NO: | | | | | | |
| T88S1 | 6 | ESVDNYGFSF | RAS | QQSNOGPLT | GFSLTSYG | IWSDGRT | ARHGRYDPYAMDY |
| | SEQ ID | 31 | 32 | 33 | 34 | 35 | 36 |
| | NO: | | | | | | |

TABLE 2
Anti-CLDN18.2 mAb heavy and light chain variable region
sequences. Antibody CDRs are underlined.

| | | SEQ |
|---|---|---|
| | | ID |
| Name | Sequence | NO: |
| T155S2 | GAAGTGAAGCTGGTGGAGTCTGGGGGAGGCTTAGTGAAGCCTGGAGGGTCCC | 37 |
| VH | TGAAACTCTCCTGTGCAGCCTCTGGATTCACTTTCAGTAGCTATGCCATGTC | |
| | TTGGGTTCGCCAGACTCCAGAGAAGAGGCTGGAGTGGGTCGCATCCATTAGT | |
| | GGTGGTGGTAGCACCTACTATCCAGACAGTGTGAAGGGCCGATTCACCATCT | |
| | CCAGAGATAATGCCAGGAACATCCTGTACCTGCAAATGAGCTATCTGAGGTC | |
| | TGAGGACACGGCCGTGTATTACTGTGCAAGAGGAGGGAATATCTATGATGGT | |
| | TACCCGTACTACTTTGACTACTGGGGCCAAGGCACCACTCTCACAGTCTCCT | |
| | CA | |
| T155S2 | EVKLVESGGGLVKPGGSLKLSCAASGFTFSSYAMSWVRQTPEKRLEWVASIS | 38 |
| VH | GGGSTYYPDSVKGRFTISRDNARNILYLQMSYLRSEDTAVYYCARGGNIYDG | |
| | YPYYFDYWGQGTTLTVSS | |
| T155S2 | GACATTGTGATGACCCAGTCTCCATCCTCCCTGGCTATGTCAGTAGGACAGA | 39 |
| VL | AGGTCACTATGAGCTGCAAGTCCAGTCAGAACCTTTTAAATAGTAGCAATCA | |
| | AAAGAACTATTTGGCCTGGTACCAGCAGAAACCAGGACAGTCTCCTAAACTT | |
| | CTGGTATACTTTGCATCCACTAGGGAATCTGGGGTCCCTGATCGCTTCACAG | |
| | GCAGTGGATCTGGGACAGATTTCACTCTTACCATCAGCAGTGTGCAGGCTGA | |
| | AGACCTGGCAGTTTATTACTGTCAGCAACATTATAGCACTCCGCTCACGTTC | |
| | GGTGCTGGGACCAAGCTGGAGCTGAAA | |
| T155S2 | DIVMTQSPSSLAMSVGQKVTMSCKSSQNLLNSSNQKNYLAWYQQKPGQSPKL | 40 |
| VL | LVYFASTRESGVPDRFTGSGSGTDFTLTISSVQAEDLAVYYCQQHYSTPLTF | |
| | GAGTKLELK | |
| T158S2 | GAAGTGAAGCTGGTGGAGTCTGGGGGAGGCTTAGTGCAGCCTGGAGGGTCCC | 41 |
| VH | TGAAACTCTCCTGTGCAACCTCTGGATTCACTTTCAGTGACTATTACATGTT | |
| | TTGGATTCGCCAGACTCCAGAGAAGAGGCTGGAGTGGGTCGCCTCCATTAGT | |
| | GATGGTGGTGGTGACACCTATTATCCAGACACTGTAAAGGGCCGATTCACCA | |
| | TCTCCAGAGACAATGCCAAGAACACCCTGTACCTGCAAATGAGCCGTCTGAA | |
| | GTCTGAGGACACAGCCATGTATTACTGTGCAAGACATAGGGGCTCTCTTGAC | |
| | TACTGGGGCCAAGGCACCACTCTCACAGTCTCCTCA | |
| T158S2 | EVKLVESGGGLVQPGGSLKLSCATSGFTFSDYYMFWIRQTPEKRLEWVASIS | 42 |
| VH | DGGGDTYYPDTVKGRFTISRDNAKNTLYLQMSRLKSEDTAMYYCARHRGSLD | |
| | YWGQGTTLTVSS | |
| T158S2 | GACATTGTGCTGACCCAATCTCCAGCTTCTTTGGCTGTGTCTCTAGGGCAGA | 43 |
| VL | GGGCCACCATCTCCTGCAGAGCCAGCGAGAGTGTGGATAACTATGGCTTTAG | |
| | TTTTCTGCACTGGTACCAGCAGAAACCAGGACAGCCACCCAAACTCCTCATC | |
| | TATCGTGCATCCAACCTAGCATCTGGGATCCCTGCCAGGTTCAGTGGCAGTG | |
| | GGTCTAGGACAGACTTCACCCTCACCATTAATCCTGTGGAGACTGATGATGT | |
| | TGCAACCTATTACTGTCAGCAAAGTAATCAGGGTCCACTCACGTTCGGTGCT | |
| | GGGACCAAGCTGGAGCTGAAA | |
| T158S2 | DIVLTQSPASLAVSLGQRATISCRASESVDNYGFSFLHWYQQKPGQPPKLLI | 44 |
| VL | YRASNLASGIPARFSGSGSRTDFTLTINPVETDDVATYYCQQSNQGPLTFGA | |
| | GTKLELK | |
| T224S8 | GAGGTTCAGCTGCAGCAGTCTGGGACAGAGCTTGTGAGGTCAGGGGCCTCAG | 45 |
| VH | TCAAGTTGTCCTGCACAACTTCTGGCTTCAACATTAAAGACTACTTTTTACA | |
| | CTGGGTGAAGCAGAGGCCTGAACAGGGCCTGGAGTGGATTGGATGGATTGAT | |
| | CCTGAGAATGGTGATACTAAATATGCCCCGAAGTTCCAGGACAAGGTCACCA | |
| | TGACTGTGGACACATCCTCCAACACAGCCTGCCTGCACCTCAGCAGCCTGAC | |
| | ATCTGATGACACTGCCGTCTATTACTGTAATGTACTAGGCTACGGTAATTAC | |
| | GGACATTTTTACTATGCTATGGACTACTGGGGTCAAGGAACCTCAGTCACCG | |
| | TCTCCTCT | |
| T224S8 | EVQLQQSGTELVRSGASVKLSCTTSGFNIKDYFLHWVKQRPEQGLEWIGWID | 46 |
| VH | PENGDTKYAPKFQDKVTMTVDTSSNTACLHLSSLTSDDTAVYYCNVLGYGNY | |
| | GHFYYAMDYWGQGTSVTVSS | |
| T224S8 | GATGTTGTGATGACCCAAACTCCACTCTCCCTGCCTGTCAGTCTTGGAGATC | 47 |
| VL | AGGCCTCCATCTCTTGCAGATCTAGTCAGAGCCTTGTACACAGTAATGGAAA | |
| | CACCTATTTACATTGGTACCTGCAGAAGCCAGGCCAGTCTCCAAAGCTCCTG | |
| | ATCTACAAAGTTTCCAACCGATTTTCTGGGGTCCCAGACAGGTTCAGTGGCA | |
| | GTGAATCAGGGACAGATTTCACACTCAAGATCAGCAGAGTGGAGGCTGAGGA | |
| | TCTGGGAATTTATTTCTGCTCTCAAAATACACATGTTCCTCGGACGTTCGGT | |
| | GGAGGCACCAAGCTGGAAATCAGA | |
| T224S8 | DVVMTQTPLSLPVSLGDQASISCRSSQSLVHSNGNTYLHWYLQKPGQSPKLL | 48 |
| VL | IYKVSNRFSGVPDRFSGSESGTDFTLKISRVEAEDLGIYFCSQNTHVPRTFG | |
| | GGTKLEIR | |
| T192S2 | GAAGTGAAGCTGGTGGAGTCTGGGGGAGGCTTAGTGCAGCCTGGAGGGTCCC | 49 |
| VH | TGAAACTCTCCTGTGCAACCTCTGGATTCACTTTCAGTGACTATTACATGTA | |
| | TTGGGTTCGCCAGACTCCAGAGAAGAGGCTGGAGTGGGTCGCATACATTAGT | |
| | AATGGTGGTGGTTCCACCTATTATCCAGACACTGTGAAGGGCCGATTCACCA | |
| | TCTCCAGAGACAATGCCAAGAACACCCTGTACCTGCAAATGAGCCGTCTGAA | |
| | GTCTGAGGACACAGCCATGTATTACTGTGCAAGACATAGGGGCTCTCTTGAC | |
| | TTCTGGGGCCAAGGCACCACTCTCACAGTCTCCTCA | |
| T192S2 | EVKLVESGGGLVQPGGSLKLSCATSGFTFSDYYMYWVRQTPEKRLEWVAYIS | 50 |
| VH | NGGGSTYYPDTVKGRFTISRDNAKNTLYLQMSRLKSEDTAMYYCARHRGSLD | |
| | FWGQGTTLTVSS | |
| T192S2 | GACATAGTGCTGACACAGTCTCCTGCTTCCTTAGCTGTATCTCTGGGGCAGA | 51 |
| VL | GGGCCACCATCTCCTGCAGAGCCAGCGAAAGTGTTGATAATTATGGCTTTAG | |
| | TTTTATGCACTGGTACCAGCAGAAACCAGGACAGCCACCCAAACTCCTCATC | |
| | TATCGTGCATCCAACCTAGAATCTGGGATCCCTGCCAGGTTCAGTGGCAGTT | |
| | GGTCTAGGACAGACTTCACCCTCACCATTAATCCTGTGGAGACTGATGATGT | |
| | TGCAACCTATTACTGTCAGCAAAGTAATAAGGTTCCGCTCACGTTCGGTGCT | |
| | GGGACCAAGCTGGAGCTGAAA | |
| T192S2 | DIVLTQSPASLAVSLGQRATISCRASESVDNYGFSFMHWYQQKPGQPPKLLI | 52 |
| VL | YRASNLESGIPARFSGSWSRTDFTLTINPVETDDVATYYCQQSNKVPLTFGA | |
| | GTKLELK | |
| T252S4 | GAGGTCCAGCTGCAACAGTCTGGACCTGAGCTGGTGAAGCCTGGAGCTTCAA | 53 |
| VH | TGAAGATATCCTGCAAGGCTTCTGGTTACTCATTCACTGGCTACAACATGAA | |
| | CTGGGTGAAGCAGACCCATGGAAAGAACCTTGAGTGGATTGGACTTATTAAT | |
| | CCTTACAATGGTGGTACTAGGTACAACCAGAAGTTCAAGGGCAAGGCCACAT | |
| | TAACTGTAGACAAGTCATCCAGCACAGCCTACATGGAGCTCCTCAGTCTGAC | |
| | ATCTGAGGACTCTGCAGTCTATTTCTGTGCAAGGATGGGACTTGGAAATGCT | |
| | ATGGACTACTGGGGTCAAGGAACCTCAGTCACCGTCTCCTCA | |
| T252S4 | EVQLQQSGPELVKPGASMKISCKASGYSFTGYNMNWVKQTHGKNLEWIGLIN | 54 |
| VH | PYNGGTRYNQKFKGKATLTVDKSSSTAYMELLSLTSEDSAVYFCARMGLGNA | |
| | MDYWGQGTSVTVSS | |
| T252S4 | GACATTGTGATGACACAGTCTCCATCCTCCCTGACTGTGACAGCAGGAGAGA | 55 |
| VL | AGGTCACTATGAGCTGCAAGTCCAGTCAGAATCTGTTAAACAGTGGAAATCA | |
| | AAAGAACTACTTGACCTGGTACCAGCAGAAACCAGGGCAGCCTCCTAAACTG | |
| | TTGATCTACTGGGCATCCACTATGGAATCTGGGGTCCCTGATCGCTTCACAG | |
| | GCAGTGGATCTGGAACAGATTTCACTCTCACCATCAGCAGTGTGCAGGCTGA | |
| | AGACCTGGCAGTTTATTACTGTCAGAATGATTATATTTATCCGCTCCCGTTC | |
| | GGTGCTGGGACCAAGCTGGAGCTGAAA | |
| T252S4 | DIVMTQSPSSLTVTAGEKVTMSCKSSQNLLNSGNQKNYLTWYQQKPGQPPKL | 56 |
| VL | LIYWASTMESGVPDRFTGSGSGTDFTLTISSVQAEDLAVYYCQNDYIYPLPF | |
| | GAGTKLELK | |
| T88S1 | CAGGTGCAGCTGAAGGAGTCAGGACCTGGCCTGGTGGCGCCCTCACAGAGCC | 57 |
| VH | TGTCCATCACATGCACCATCTCAGGGTTCTCATTAACCAGCTATGGTATACA | |
| | CTGGGTTCGCCAGCCTCCAGGAAAGGGTCTGGAGTGGCTGGTAGTGATATGG | |
| | AGTGATGGAAGAACAACCTATAATTCAGCTCTCAAATCCAGACTGAGCATCA | |
| | GCAAGGACAACTCCAAGAGCCAAGTTTTCTTAAAAATGAACAGTCTCCAAAC | |
| | TGATGACACAGCCATGTACTACTGTGCCAGACATGGTAGATACGACCCCTAT | |
| | GCTATGGACTACTGGGGTCAAGGAACCTCAGTCACCGTCTCCTCA | |
| T88S1 | QVQLKESGPGLVAPSQSLSITCTISGFSLTSYGIHWVRQPPGKGLEWLVVIW | 58 |
| VH | SDGRTTYNSALKSRLSISKDNSKSQVFLKMNSLQTDDTAMYYCARHGRYDPY | |
| | AMDYWGQGTSVTVSS | |
| T88S1 | GACATTGTGCTGACCCAATCTCCAGCTTCTTTGGCTGTGTCTCTAGGGCAGA | 59 |
| VL | GGGCCACCATCTCCTGCAGAGCCAGCGAGAGTGTGGATAACTATGGCTTTAG | |
| | TTTTCTGCACTGGTACCAGCAGAAACCAGGACAGCCACCCAAACTCCTCATC | |
| | TATCGTGCATCCAACCTAGCATCTGGGATCCCTGCCAGGTTCAGTGGCAGTG | |
| | GGTCTAGGACAGACTTCACCCTCACCATTAATCCTGTGGAGACTGATGATGT | |
| | TGCAACCTATTACTGTCAGCAAAGTAATCAGGGTCCACTCACGTTCGGTGCT | |
| | GGGACCAAGCTGGAGCTGAAA | |
| T88S1 | DIVLTQSPASLAVSLGQRATISCRASESVDNYGFSFLHWYQQKPGQPPKLLI | 60 |
| VL | YRASNLASGIPARFSGSGSRTDFTLTINPVETDDVATYYCQQSNQGPLTFGA | |
| | GTKLELK | |

### Example 2: Anti-CLDN18.2 Mouse Monoclonal Antibody Binding Specificity and Affinity

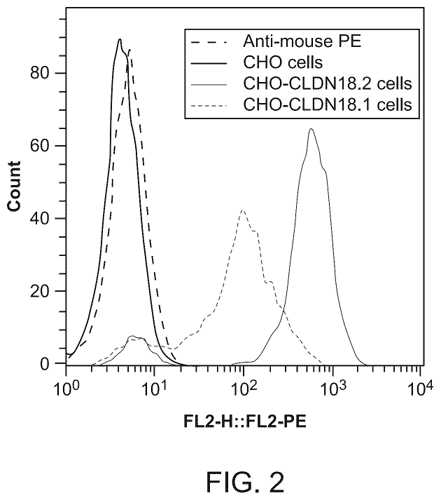

To evaluate the binding specificity of anti-CLDN18.2 mouse hybridoma antibodies, the purified antibodies were analyzed by FACS analysis using CHO cells expressing either human CLDN18.1 or human CLDN18.2. The FACS analysis was done as described previously. As shown in FIGS. 1a and 1b, all 9 hybridoma antibodies bind to CHO cells expressing human CLDN18.2 but not to CHO cells expressing human CLDN18.1. One of the hybridoma antibody T29S9 bind to both CLDN18.1 and 18.2 (FIG. 2).

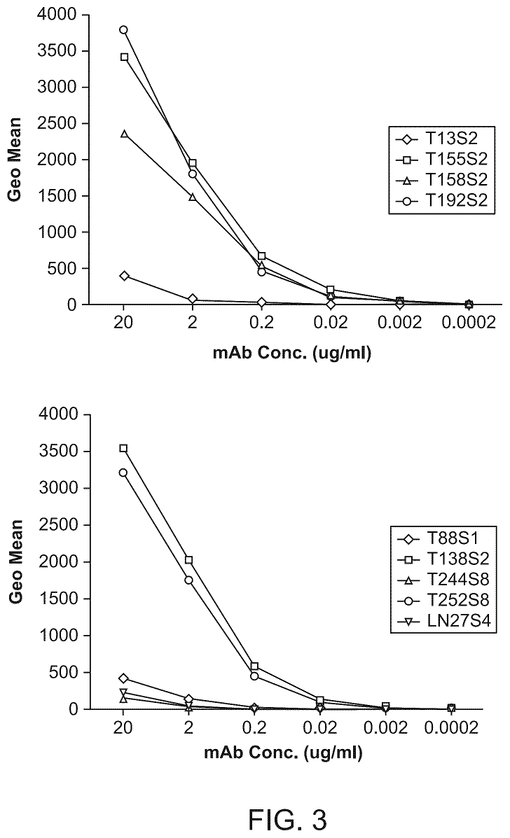

To evaluate the binding affinity of anti-CLDN18.2 mouse hybridoma antibodies to cell surface expressed human CLDN18.2, serial dilutions of anti-CLDN18.2 purified antibodies were analyzed by FACS using CHO cells expressing human CLDN18.2. As shown in FIG. 3, some of the antibodies had high binding affinity to cell surface CLDN18.2, such as T138S2, T155S2, T158S2, T192S2, and T252S4 while others had medium to low binding affinities, such as T13S2, T88S1, T224S8 and LN27S4.

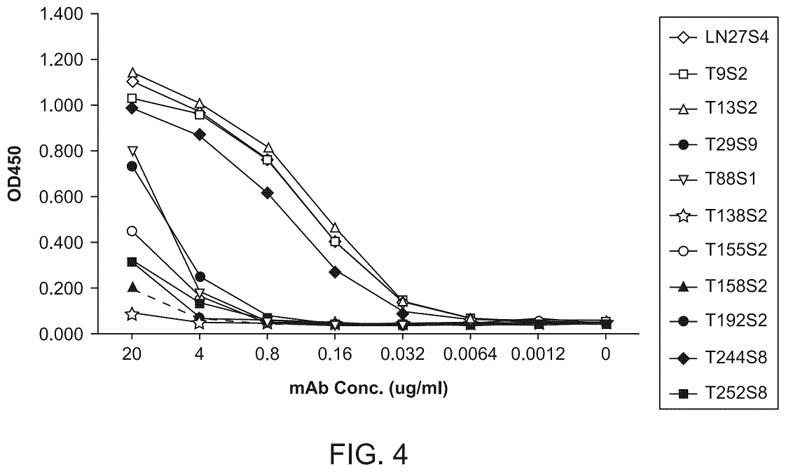

The binding of anti-human CLDN18.2 antibody to human CLDN18.2 can also be evaluated in ELISA assays. Briefly, 96 well microtiter plates were coated with human CLDN18.2 huFc protein at 1μ/ml in PBS, 100 μl/well at 4° C. overnight and then blocked with 100 μl/well of 2% BSA at RT for 1 hour. Five-fold dilutions of purified mouse hybridoma antibodies starting from 20 μg/ml were added to each well and incubated for 1-2 hours at RT. The plates were washed with PBS/Tween and then incubate with goat-anti-mouse IgG antibody conjugated with Horse Radish Peroxidase (HRP) for 1 hour at RT. After washing, the plates were developed with TMB substrate and analyzed by spectrophotometer at OD 450 nm. As shown in FIG. 4, all antibodies can bind to plated coated human CLDN18.2 protein at various degrees. It was interesting to observe that some of the antibodies which bind to cell surface CLDN18.2 with high affinities, such as T138S2 and T158S2, did not bind to plate-coated CLDN18.2 very well (FIG. 4).

### Example 3: Anti-CLDN18.2 Mouse Monoclonal Antibody Binding to KATO III Cells

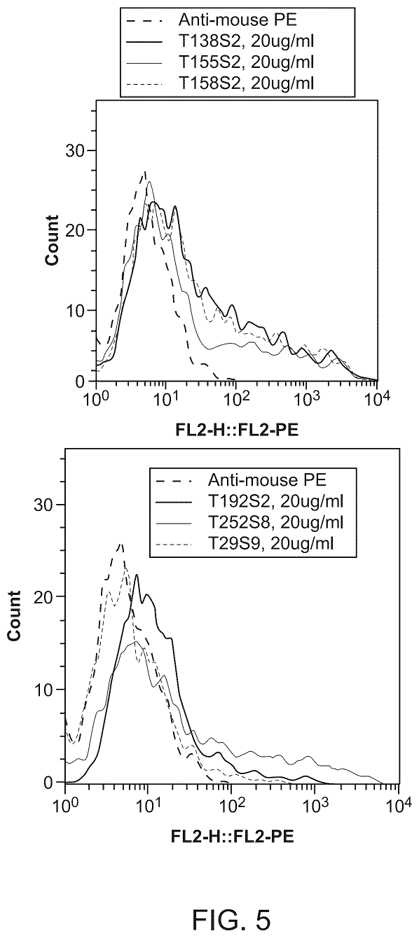

FACS assay was also used to evaluate the binding of anti-CLDN18.2 antibodies to human gastric cancer cell line KATO III. As shown in FIG. 5, the six anti-CLDN18.2 hybridoma antibodies can bind to KATO III cells at various levels.

### Example 4: Anti-Human CLDN18.2 Mouse/Human Chimeric Antibody Binding Affinity to Human CLDN18.2 and KATO III Cells

To produce anti-human CLDN18.2 mouse/human chimeric antibodies, mouse antibody heavy and light chain V regions were cloned into transient expression vectors containing human antibody heavy chain IgG1 and light chain constant regions. The resulting antibody heavy and light chain expression constructs were used to transfect HEK293 cells. The culture supernatants were harvested and loaded onto protein A Sepharose columns (GE Healthcare). The columns were washed and antibodies were then eluted with eluting buffer (0.1 M glycine buffer, pH 3.0). Collected fractions were neutralized with 1 MTris-HCl, pH 8.0, pooled together and then dialyzed against PBS. Purity of the antibodies was analyzed by sodium dodecyl sulfate polyacrylamide gel electrophoresis (SDS-PAGE) under both reducing and non-reducing conditions. Protein bands were visualized by Coomassie brilliant blue staining.

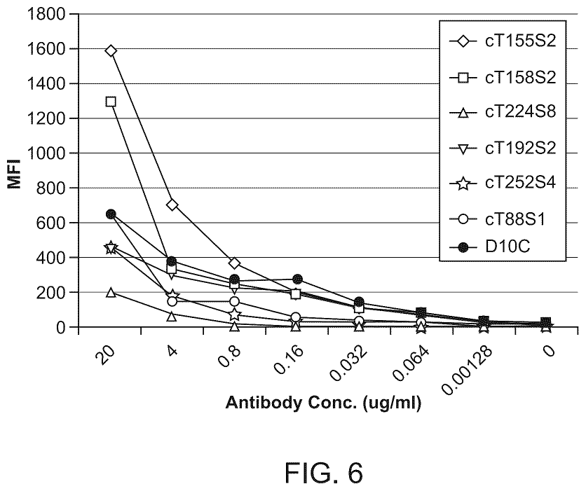

To evaluate the binding affinity of anti-human CLDN18.2 mouse/human chimeric antibodies to human CLDN18.2, CHO cells expressing human CLDN18.2 were first incubated with five fold serial diluted mouse/human chimeric mAbs starting at 100 μg/ml on ice for 30 min. The cells were then washed with PBS three times, followed by incubation with PE-labeled anti-human Fc gamma specific antibody (Jackson Immuno-Research) at 4° C. for 15 min. The cells were washed again with PBS three times and analyzed using an FACS Caliber instrument (Becton-Dickinson). As shown in FIG. 6, all six chimeric antibodies bind to CHO-CLDN18.2 cells at similar affinity as their hybridoma counterparts. D10C is an anti-human CLDN18.2 antibody identified by GanyMed and is used as a control.

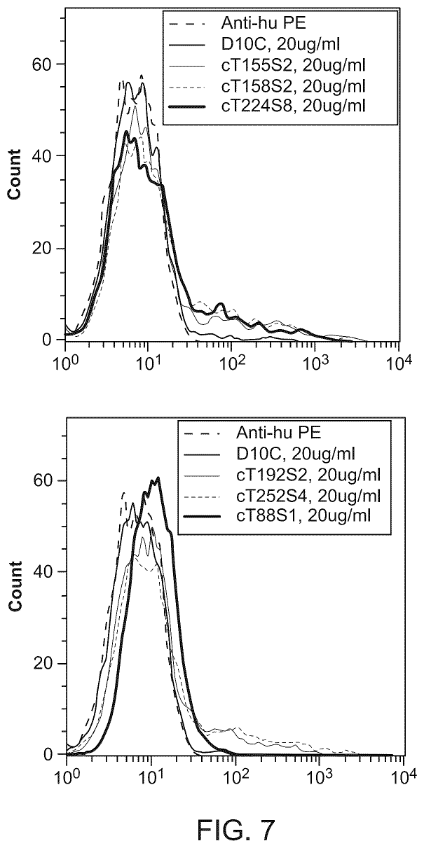

The binding affinities of anti-human CLDN18.2 chimeric antibodies to human gastric cancer cell line KATO III were also evaluated by FACS. As shown in FIG. 7, the anti-CLDN18.2 chimeric antibodies bound to only a small fraction of KATO III cells. However, most of the antibodies tested bound to KATO III cells at higher levels compared to the D10C antibody (FIG. 7).

### Example 5: Anti-Human CLDN18.2 Antibody-Induced Receptor Internalization in Target Cells

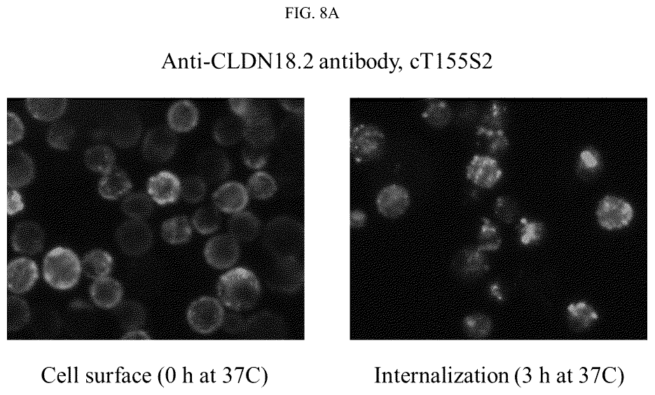

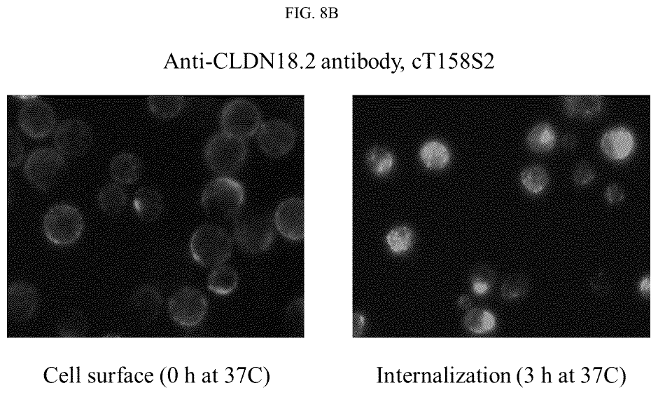

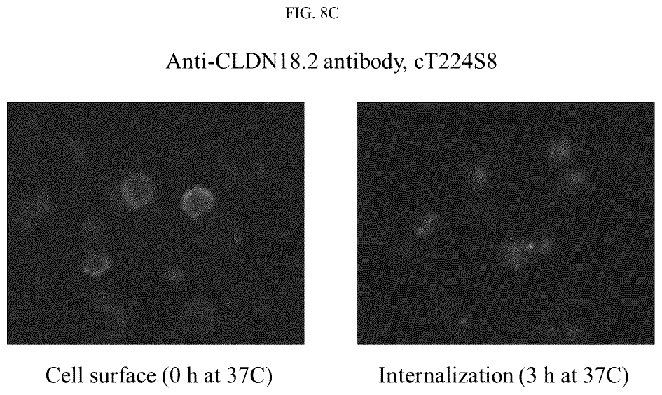

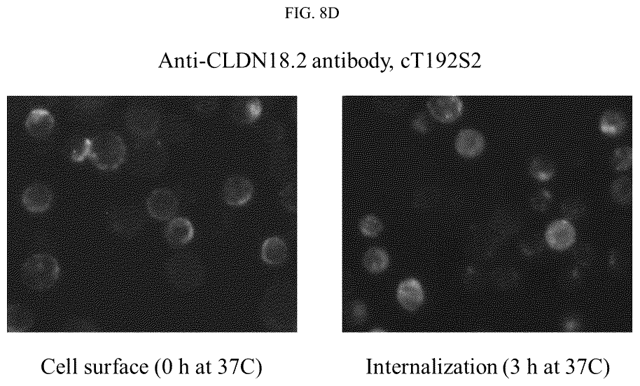

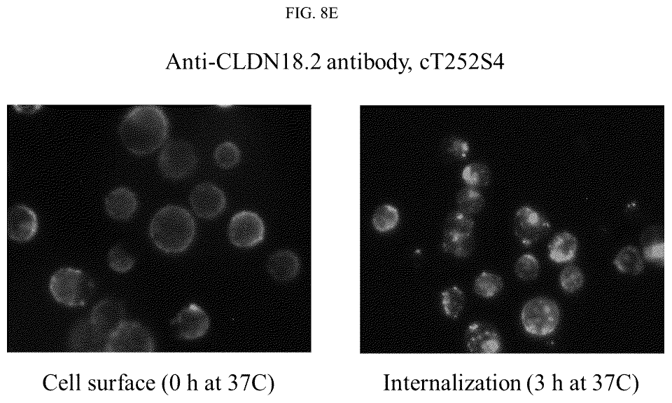

The anti-human CLDN18.2 antibody-induced CLDN18/2 internalization was evaluated. Anti-human CLDN18.2 antibodies, cT155S2, cT158S2, cT224S8, cT192S2, and cT252S4, were conjugated with FITC per manufacturer's instructions (Sigma). CHO-CLDN18.2 cells were stained with the FITC-labeled antibodies at 4° C. for 20 min and washed with PBS twice, followed by incubation at 37° C. for 0 hr (for cell surface binding) or 3 hr (for antibody internalization). The cells were then analyzed by fluorescence microscopy. As shown in FIGS. 8a-8e, all of the tested anti-human CLDN18.2 antibodies, including cT155S2, cT158S2, cT224S8, cT192S2, and cT252S4, effectively induced CLDN18.2 internalization on CHO-CLDN18.2 cells (FIGS. 8a-8c). These data demonstrated that the anti-CLDN18.2 antibodies conjugated with a cytotoxin or cytotoxic agents can be internalized by target cancer cells expressing CLDN18.2. Thus the anti-CLDN18.2 antibody-toxin conjugates can be used as antibody-drug conjugates (ADCs) for treatment of cancer and other diseases.

### Example 6: Humanization of Mouse Anti-Human CLDN18.2 Antibody T252S4

The mAb T252S4 variable region genes were employed to create a humanized MAb. In the first step of this process, the amino acid sequences of the VH and VK of MAb T252S4 were compared against the available database of human Ig gene sequences to find the overall best-matching human germline Ig gene sequences. For the light chain, the closest human match was the IGKV4-1 gene, and for the heavy chain the closest human match was the IGVH5-51 gene.

Humanized variable domain sequences were then designed where the CDR1, CDR2, and CDR3 sequences (underlined sequences in Table 3) of the T252S4 light chain were grafted onto framework sequences of the IGKV4-1 gene, and the CDR1, CDR2, and CDR3 sequences (underlined sequences in Table 3) of the T252S4 heavy chain were grafted onto framework sequences of the IGVH5-51. A 3D model was then generated to determine if there were any framework positions where replacing the mouse amino acid to the human amino acid could affect binding and/or CDR conformation. The amino acid and nucleotide sequences of some of the humanized antibody are listed in Table 3 below.

TABLE 3
Humanized T252S4antibody sequences (CDRs are underlined)

| | | SEQ ID |
|---|---|---|
| Name | Sequence | NO: |
| Chimeric | EVQLQQSGPELVKPGASMKISCKASGYSFT GYNMN WVKQTHGKNLEWIG | 61 |
| T252S4 VH | LINPYNGGTRYNQKFKG KATLTVDKSSSTAYMELLSLTSEDSAVYFCAR | |
| | MGLGNAMDY WGQGTSVTVSS | |
| HuT252S4 VH1 | EVQLVQSGAEVKKPGESLKISCKGSGYSFT GYNMN WVRQMPGKGLEWMG | 62 |
| | LINPYNGGTRYNQKFKG QVTISADKSISTAYLQWSSLKASDTAMYFCAR | |
| | MGLGNAMDY WGQGTTVTVSS | |
| HuT252S4 VH2 | EVQLVQSGAEVKKPGESLKISCKGSGYSFT GYNMN WVRQMPGKNLEWIG | 63 |
| | LINPYNGGTRYNQKFKG QVTISADKSISTAYLQWSSLKASDTAMYFCAR | |
| | MGLGNAMDY WGQGTTVTVSS | |
| HuT252S4 VH3 | EVQLVQSGAEVKKPGESLKISCKGSGYSFT GYNMN WVRQMPGKNLEWIG | 64 |
| | LINPYNGGTRYNQKFKG KATLSVDKSISTAYLQWSSLKASDTAMYFCAR | |
| | MGLGNAMDY WGQGTTVTVSS | |
| Chimeric | DIVMTQSPSSLTVTAGEKVTMSC KSSQNLLNSGNQKNYLT WYQQKPGQPPKLLIY | 65 |
| T252S4 VL | WASTMES GVPDRFTGSGSGTDFTLTISSVQAEDLAVYYC QNDYIYPLP | |
| | FGAGTKLELK | |
| HuT252S4 VL1 | DIVMTQSPDSLAVSLGERATINC KSSQNLLNSGNQKNYLT WYQQKPGQPPKLLIY | 66 |
| | WASTMES GVPDRFSGSGSGTDFTLTISSLQAEDVAVYYC QNDYIYPLP | |
| | FGQGTKLEIK | |
| HuT252S4 VL2 | DIVMTQSPDSLAVSLGERATINC KSSQNLLQSGNQKNYLT WYQQKPGQPPKLLIY | 67 |
| | WASTRES GVPDRFSGSGSGTDFTLTISSLQAEDVAVYYC QNDYIYPLP | |
| | FGQGTKLEIK | |
| Chimeric | GAGGTCCAGCTGCAACAGTCTGGACCTGAGCTGGTGAAGCCTGGAGCTTCAATGAAGAT | 68 |
| T252S4 VH | ATCCTGCAAGGCTTCTGGTTACTCATTCACTGGCTACAACATGAACTGGGTGAAGCAGA | |
| | CCCATGGAAAGAACCTTGAGTGGATTGGACTTATTAATCCTTACAATGGTGGTACTAGG | |
| | TACAACCAGAAGTTCAAGGGCAAGGCCACATTAACTGTAGACAAGTCATCCAGCACAGC | |
| | CTACATGGAGCTCCTCAGTCTGACATCTGAGGACTCTGCAGTCTATTTCTGTGCAAGGA | |
| | TGGGACTTGGAAATGCTATGGACTACTGGGGTCAAGGAACCTCAGTCACCGTCTCCTCA | |
| HuT252S4 VH1 | GAAGTCCAGCTCGTGCAGTCTGGTGCGGAGGTCAAAAAACCCGGCGAGTCTCTCAAAAT | 69 |
| | TAGTTGCAAAGGCTCCGGTTATTCATTCACGGGTTACAATATGAACTGGGTTCGACAAA | |
| | TGCCGGGTAAGGGACTTGAATGGATGGGATTGATCAACCCCTATAACGGAGGTACCAGA | |
| | TACAACCAAAAATTTAAGGGCCAGGTGACTATTAGTGCAGATAAAAGTATCTCCACCGC | |
| | TTACCTCCAATGGTCTAGTTTGAAGGCGTCTGACACTGCTATGTACTTTTGCGCGAGAA | |
| | TGGGGTTGGGAAATGCGATGGACTACTGGGGTCAGGGCACTACGGTTACGGTCTCCTCT | |
| HuT252S4 VH2 | GAGGTACAGCTCGTGCAATCTGGGGCCGAAGTAAAGAAACCTGGAGAAAGTCTTAAAAT | 70 |
| | AAGCTGCAAGGGCAGTGGGTATAGCTTCACGGGCTACAATATGAACTGGGTTAGACAAA | |
| | TGCCAGGTAAGAACCTCGAATGGATAGGATTGATCAACCCATACAATGGTGGTACGCGC | |
| | TACAACCAGAAATTCAAAGGTCAGGTTACCATCTCTGCGGATAAAAGCATCTCAACGGC | |
| | GTATCTTCAATGGTCCTCACTGAAAGCATCCGACACAGCAATGTATTTTTGCGCTAGAA | |
| | TGGGATTGGGTAATGCCATGGACTACTGGGGCCAAGGCACCACGGTTACAGTCTCTTCC | |
| HuT252S4 VH3 | GAAGTTCAGCTGGTGCAAAGTGGGGCTGAGGTAAAGAAGCCTGGCGAGAGCCTCAAGAT | 71 |
| | TTCTTGCAAGGGTAGCGGATACAGTTTTACGGGATATAACATGAATTGGGTTCGCCAGA | |
| | TGCCGGGGAAGAACCTTGAATGGATAGGACTGATAAACCCCTATAATGGGGGAACCCGA | |
| | TATAATCAGAAGTTTAAGGGAAAAGCAACTTTGTCAGTTGACAAGTCTATCAGCACGGC | |
| | CTATCTTCAGTGGTCCAGTCTGAAAGCAAGCGACACGGCTATGTACTTTTGTGCACGCA | |
| | TGGGGCTTGGTAACGCAATGGACTATTGGGGACAAGGAACTACCGTCACTGTCTCTTCA | |
| Chimeric | GACATTGTGATGACACAGTCTCCATCCTCCCTGACTGTGACAGCAGGAGAGAAGGTCAC | 72 |
| T252S4 VL | TATGAGCTGCAAGTCCAGTCAGAATCTGTTAAACAGTGGAAATCAAAAGAACTACTTGA | |
| | CCTGGTACCAGCAGAAACCAGGGCAGCCTCCTAAACTGTTGATCTACTGGGCATCCACT | |
| | ATGGAATCTGGGGTCCCTGATCGCTTCACAGGCAGTGGATCTGGAACAGATTTCACTCT | |
| | CACCATCAGCAGTGTGCAGGCTGAAGACCTGGCAGTTTATTACTGTCAGAATGATTATA | |
| | TTTATCCGCTCCCGTTCGGTGCTGGGACCAAGCTGGAGCTGAAA | |
| HuT252S4 VL1 | GACATCGTGATGACCCAGTCCCCGGACAGTCTGGCAGTCAGTCTTGGGGAAAGAGCTAC | 73 |
| | CATAAACTGCAAATCCAGCCAAAACCTTCTTAATAGCGGCAACCAAAAGAATTACTTGA | |
| | CTTGGTATCAGCAAAAACCGGGTCAGCCGCCCAAACTCTTGATATACTGGGCGTCTACG | |
| | ATGGAAAGCGGCGTCCCCGACCGCTTCAGCGGGAGTGGGTCAGGGACTGATTTCACTTT | |
| | GACAATCAGTTCCCTTCAGGCAGAGGACGTAGCAGTCTACTACTGTCAGAATGATTATA | |
| | TATATCCTCTTCCGTTCGGCCAGGGGACGAAGTTGGAGATCAAA | |
| HuT252S4 VL2 | GACATCGTGATGACTCAAAGTCCTGACTCCCTTGCTGTTTCACTTGGCGAAAGGGCCAC | 74 |
| | TATCAACTGTAAGAGTTCTCAGAATCTCTTGCAATCAGGAAACCAGAAGAATTACTTGA | |
| | CCTGGTATCAACAGAAGCCTGGACAACCACCTAAGCTCTTGATTTACTGGGCTAGTACA | |
| | AGGGAGTCCGGCGTCCCAGACAGATTTTCCGGTTCTGGATCAGGCACGGACTTCACTCT | |
| | GACAATCTCTAGTCTTCAAGCCGAGGATGTGGCCGTTTATTATTGCCAGAACGATTACA | |
| | TTTACCCTTTGCCATTTGGTCAAGGTACTAAGTTGGAGATAAAA | |

The humanized VH and VK genes were produced synthetically and then respectively cloned into vectors containing the human gamma 1 and human kappa constant domains. The pairing of the human VH and the human VK created 6 humanized antibodies (see Table 4).

TABLE 4
Humanized T252S4 antibodies with their VH and VL regions

| | | | |
|---|---|---|---|
| VL | Chimeric | HuT252S4VL1 | HuT252S4VL2 |
| VH | T252S4VL | | |
| Chimeric | Chimeric | | |
| T252S4VH | T252S4 H/L | | |
| HuT252S4VH1 | | HuT252S4 H1/L1 | HuT252S4 H1/L2 |
| HuT252S4VH2 | | HuT252S4 H2/L1 | HuT252S4 H2/L2 |
| HuT252S4VH3 | | HuT252S4 H3/L1 | HuT252S4 H3/L2 |

### Example 7: Humanized T252S4 Antibodies Specifically Bind to Cell Surface Human CLDN18.2 on CHO Cells Stably Expressing CLDN18.2

To evaluate the binding activities of the humanized T252S4 antibodies to human CLDN18.2 on cell surface, humanized T252S4 antibodies were produced from HEK293 cells. Briefly, the three HuT252S4 antibody heavy chain V regions (H1, H2, and H3) and two light chain V regions (L1 and L2) were paired in different combinations (Table 5) to transfect HEK293 cells, resulting in six humanized T252S4 antibodies. The chimeric T252S4 H/L was also expressed for comparison. The culture supernatants were harvested and loaded onto protein A Sepharose columns (GE Healthcare). The columns were washed and antibodies were then eluted with eluting buffer (0.1 M glycine buffer, pH 3.0). Collected fractions were neutralized with 1 M Tris-HCl, pH 8.0, pooled together and then dialyzed against PBS. Purity of the antibodies was analyzed by sodium dodecyl sulfate polyacrylamide gel electrophoresis (SDS-PAGE) under both reducing and non-reducing conditions.

To evaluate the binding activities of the humanized T252S4 antibodies to cell surface-expressed human CLDN18.2, serial dilutions of purified humanized anti-CLDN18.2 antibodies were analyzed by FACS using CHO cells expressing human CLDN18.2. As shown in FIG. 9, all humanized T252S4 antibodies, including HuT252S4 H1/L1, HuT252S4H2/L1, HuT252S4H3/L1, HuT252S4H1/L2, HuT252S4H2/L2, and HuT252S4H3/L2, had high binding affinities to cell surface CLDN18.2. The humanized T252S4 antibodies, with EC50 ranging from 0.47 to 1 μg/ml, had similar affinities with or higher affinities than the chimeric T252S4 antibody (EC50 at 1 μg/ml) binding to cell surface CLDN18.2 (FIG. 9).

### EQUIVALENTS

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

What is claimed is:

1. An isolated antibody or fragment thereof comprising a heavy chain (HC) immunoglobulin variable domain sequence and a light chain (LC) immunoglobulin variable domain sequence, wherein the antibody binds to an epitope of CLDN 18.2, and does not bind to CLDN 18.1, wherein the HC comprises one or more of:
(a) a CDR1 comprising an amino acid sequence selected from: (i) GYSFTGYN (SEQ ID NO: 28), (ii) GFTFSDYY (SEQ ID NO: 10), (iii) GFNIKDYF (SEQ ID NO: 16), (iv) GFSLTSYG (SEQ ID NO: 34), or (v) GYNMN (SEQ ID NO: 93), or an equivalent of each thereof;
(b) a CDR2 comprising an amino acid sequence selected from: (i) INPYNGGT (SEQ ID NO: 29), (ii) ISDGGGDT (SEQ ID NO: 11), (iii) IDPENGDT (SEQ ID NO: 17), (iv) ISNGGGST (SEQ ID NO: 23), (v) IWSDGRT (SEQ ID NO: 35), (vi) LINPYNGGTRYNQKFKG (SEQ ID NO: 94), or an equivalent of each thereof; and/or
(c) a CDR3 comprising an amino acid sequence selected from: (i) ARMGLGNAMDY (SEQ ID NO: 30), (ii) ARHRGSLDY (SEQ ID NO: 12), (iii) NVLGYGNYGHFYYAMDY (SEQ ID NO: 18), (iv) ARHRGSLDF (SEQ ID NO: 24), (v) ARGGNIYDGYPYYFDY (SEQ ID NO: 95), (vi) ARHGRYDPYAMDY (SEQ ID NO: 36), (vi) MGLGNAMDY (SEQ ID NO: 96), or an equivalent of each thereof;
the LC comprises one or more of:
(a) a CDR1 comprising an amino acid sequence selected from: (i) QNLLNSGNQKNY (SEQ ID NO: 25), (ii) ESVDNYGFSF (SEQ ID NO: 7), (iii) QSLVHSNGNTY (SEQ ID NO: 13), (iv) KSSQNLLNSGNQKNYLT (SEQ ID NO: 97) or an equivalent of each thereof;
(b) a CDR2 comprising an amino acid sequence selected from: (i) WAS (SEQ ID NO: 26), (ii) RAS (SEQ ID NO: 8), (iii) KVS (SEQ ID NO: 14), (iv) WASTMES (SEQ ID NO: 98), or an equivalent of each thereof; and/or
(c) a CDR3 comprising an amino acid sequence selected from: (i) QNDYTYPLP (SEQ ID NO: 27), (ii) QQSNQGPLT (SEQ ID NO: 9), (iii) SQNTHVPRT (SEQ ID NO: 15), (iv) QQSNKVPLT (SEQ ID NO: 21), or an equivalent of each thereof.

2. The antibody or fragment thereof of claim 1, wherein the HC variable region comprises at least one of:
(a)
(SEQ ID NO: 54)
EVQLQQSGPELVKPGASMKISCKASGYSFTGYNMNWVKQTHGKNLEWIGL
INPYNGGTRYNQKFKGKATLTVDKSSSTAYMELLSLTSEDSAVYFCARMG
LGNAMDYWGQGTSVTVSS,
(b)
(SEQ ID NO: 42)
EVKLVESGGGLVQPGGSLKLSCATSGFTFSDYYMFWIRQTPEKRLEWVAS
ISDGGGDTYYPDTVKGRFTISRDNAKNTLYLQMSRLKSEDTAMYYCARHR
GSLDYWGQGTTLTVSS,
(c)
(SEQ ID NO: 46)
EVQLQQSGTELVRSGASVKLSCTTSGFNIKDYFLHWVKQRPEQGLEWIGW
IDPENGDTKYAPKFQDKVTMTVDTSSNTACLHLSSLTSDDTAVYYCNVLG
YGNYGHFYYAMDYWGQGTSVTVSS,
(d)
(SEQ ID NO: 50)
EVKLVESGGGLVQPGGSLKLSCATSGFTFSDYYMYWVRQTPEKRLEWVAY
ISNGGGSTYYPDTVKGRFTISRDNAKNTLYLQMSRLKSEDTAMYYCARHR
GSLDFWGQGTTLTVSS,
(e)
(SEQ ID NO: 58)
QVQLKESGPGLVAPSQSLSITCTISGFSLTSYGIHWVRQPPGKGLEWLVV
IWSDGRTTYNSALKSRLSISKDNSKSQVFLKMNSLQTDDTAMYYCARHGR
YDPYAMDYWGQGTSVTVSS,
(f)
(SEQ ID NO: 62)
EVQLVQSGAEVKKPGESLKISCKGSGYSFTGYNMNWVRQMPGKGLEWMGL
INPYNGGTRYNQKFKGQVTISADKSISTAYLQWSSLKASDTAMYFCARMG
LGNAMDY WGQGTTVTVSS,
(g)
(SEQ ID NO: 63)
EVQLVQSGAEVKKPGESLKISCKGSGYSFT GYNMNWVRQMPGKNLEWIG
LINPYNGGTRYNQKFKGQVTISADKSISTAYLQWSSLKASDTAMYFCAR
MGLGNAMDYWGQGTTVTVSS,
or an equivalent of each thereof; and
wherein the LC variable region comprises at least one of:
(a)
(SEQ ID NO: 56)
DIVMTQSPSSLTVTAGEKVTMSC KSSQNLLNSGNQKNYLTWYQQKPGQP
PKLLIYWASTMESGVPDRFTGSGSGTDFTLTISSVQAEDLAVYYCQNDYI
YPLP FGAGTKLELK,
(b)
(SEQ ID NO: 44)
DIVLTQSPASLAVSLGQRATISCRASESVDNYGFSFLHWYQQKPGQPPKL
LIYRASNLASGIPARFSGSGSRTDFTLTINPVETDDVATYYCQQSNQGPL
TFGAGTKLELK,
(c)
(SEQ ID NO: 48)
DVVMTQTPLSLPVSLGDQASISCRSSQSLVHSNGNTYLHWYLQKPGQSPK
LLIYKVSNRFSGVPDRFSGSESGTDFTLKISRVEAEDLGIYFCSQNTHVP
RTFGGGTKLEIR,
(d)
(SEQ ID NO: 52)
DIVLTQSPASLAVSLGQRATISCRASESVDNYGFSFMHWYQQKPGQPPKL
LIYRASNLESGIPARFSGSWSRTDFTLTINPVETDDVATYYCQQSNKVPL
TFGAGTKLELK,
(e)
(SEQ ID NO: 66)
DIVMTQSPDSLAVSLGERATINC KSSQNLLNSGNQKNYLTWYQQKP
GQPPKLLIYWASTMESGVPDRFSGSGSGTDFTLTISSLQAEDVAVYYC QN
DYIYPLP FGQGTKLEIK,
or an equivalent of each thereof.

3. The antibody or fragment thereof of claim 1, wherein the antibody comprises
(a) a heavy chain variable region comprising the amino acid sequence of SEQ ID NO: 54 or an equivalent thereof, and a light chain variable region comprising the amino acid sequence of SEQ ID NO: 56 or an equivalent thereof;
(b) a heavy chain variable region comprising the amino acid sequence of SEQ ID NO: 62 or an equivalent thereof, and a light chain variable region comprising the amino acid sequence of SEQ ID NO: 66 or an equivalent thereof;
(c) a heavy chain variable region comprising the amino acid sequence of SEQ ID NO: 62 or an equivalent thereof, and a light chain variable region comprising the amino acid sequence of SEQ ID NO: 67 or an equivalent thereof;
(d) a heavy chain variable region comprising the amino acid sequence of SEQ ID NO: 63 or an equivalent thereof, and a light chain variable region comprising the amino acid sequence of SEQ ID NO: 66 or an equivalent thereof;
(e) a heavy chain variable region comprising the amino acid sequence of SEQ ID NO: 63 or an equivalent thereof, and a light chain variable region comprising the amino acid sequence of SEQ ID NO: 67 or an equivalent thereof;
(f) a heavy chain variable region comprising the amino acid sequence of SEQ ID NO: 64 or an equivalent thereof, and a light chain variable region comprising the amino acid sequence of SEQ ID NO: 66 or an equivalent thereof; or
(g) a heavy chain variable region comprising the amino acid sequence of SEQ ID NO: 64 or an equivalent thereof, and a light chain variable region comprising the amino acid sequence of SEQ ID NO: 67 or an equivalent thereof.

4. The antibody or fragment thereof of claim 1, wherein the antibody or fragment thereof is selected from the group consisting of a monoclonal antibody, a bispecific antibody, chimeric or humanized antibody and/or is comprised in an immunoconjugate or an antibody drug conjugate (ADC) which comprises the antibody or fragment thereof and an additional agent.

5. The antibody or fragment thereof of claim 4, wherein the bispecific antibody has a binding specific to a second target protein selected from the group of IL-1, CD3, CD16, CD19, CD20, CD28, CD64, PD-1, PD-L1, CTLA-4, LAG-3, CD28, CD122, 4-1BB, TIM3, OX-40, OX40L, CD40, CD40L, LIGHT, ICOS, ICOSL, GITR, GITRL, TIGIT, CD27, VISTA, B7H3, B7H4, HEVM, BTLA and KIR, or wherein the additional agent is selected from the group of a toxin, a drug, a therapeutic agent, a prodrug, a peptide, a protein, an enzyme, a virus, a lipid, a biological response modifier, a pharmaceutical agent, or polyethylene glycol (PEG).

6. The antibody or fragment thereof of claim 1, wherein an equivalent comprises a polypeptide having at least 80%, or at least 85%, or at least 90%, or at least 95% amino acid identity to polypeptide or a polypeptide that is encoded by a polynucleotide that hybridizes under conditions of high stringency to the complement of a polynucleotide encoding the polypeptide.

7. An isolated polynucleotide encoding the antibody or fragment thereof of claim 1.

8. The isolated polynucleotide of claim 7, further comprising one or more of the following: a Kozak consensus sequence located upstream of the antigen binding domain of the anti-CLDN 18.2 antibody, an enhancer, or an antibiotic resistance polynucleotide.

9. A host cell comprising the isolated polynucleotide of claim 7.

10. A composition comprising the antibody or fragment thereof of claim 1.

11. A composition comprising the isolated polynucleotide of claim 7.

12. A chimeric antigen receptor (CAR) comprising: (a) an antigen binding domain of an CLDN 18.2 antibody or fragment thereof of claim 1, (b) a CD8 α hinge domain; (c) a CD8 α transmembrane domain; (d) a 4-1BB costimulatory signaling region; and (e) a CD3 zeta signaling domain.

13. The CAR of claim 12, further comprising a linker polypeptide located between the anti-CLDN 18.2 HC variable region and the anti-CLDN 18.2 LC variable region.

14. An isolated polynucleotide encoding the CAR of claim 12.

15. A method of inhibiting the growth of a tumor that expresses or overexpresses CLDN18.2 in a subject in need thereof, comprising administering to the subject an effective amount of the antibody or fragment thereof of claim 1.

16. A method of treating a cancer that expresses or overexpresses CLDN 18.2 in a patient in need thereof, comprising administering to the subject an effective amount of the antibody or fragment thereof of claim 1.

17. A method for determining if a patient is likely to respond or is not likely to respond to an anti-CLDN 18.2 therapy, comprising contacting a cancer cell sample isolated from the patient with an effective amount of the antibody or fragment thereof of claim 1, and detecting the presence of any antibody or fragment thereof bound to the sample, wherein the presence of antibody or fragment thereof bound to the sample indicates that the patient is likely to respond to the anti-CLDN 18.2 therapy and the absence of antibody or fragment thereof bound to the sample indicates that the patient is not likely to respond to the anti-CLDN 18.2 therapy.

18. A method of detecting a pathological cell in a sample isolated from a subject, comprising:
(a) detecting the level of CLDN 18.2 in a biological sample from the subject by detecting a complex formed by the antibody or fragment thereof of claim 1; and
(b) comparing the level of CLDN 18.2 observed in step (a) with the levels of CLDN 18.2 observed in a control biological sample;
wherein the pathological cell is detected when the level of CLDN 18.2 is elevated compared to that observed in the control biological sample, optionally wherein the detection comprises one or more of immunohistochemistry (IHC), Western Blotting, Flow cytometry or ELISA.

19. The antibody or fragment thereof of claim 1, wherein
the HC comprises a CDR1 comprising an amino acid sequence of GYSFTGYN (SEQ ID NO: 28) or an equivalent thereof, a CDR2 comprising an amino acid sequence of INPYNGGT (SEQ ID NO: 29) or an equivalent thereof, and a CDR3 comprising an amino acid sequence of ARMGLGNAMDY (SEQ ID NO: 30) or an equivalent thereof; and
the LC comprises a CDR1 comprising an amino acid sequence of QNLLNSGNQKNY (SEQ ID NO: 25) or an equivalent thereof, a CDR2 comprising an amino acid sequence of WAS (SEQ ID NO: 26) or an equivalent thereof, and a CDR3 comprising an amino acid sequence of QNDYIYPLP (SEQ ID NO: 27) or an equivalent thereof.

20. An isolated antibody or fragment thereof comprising a heavy chain (HC) immunoglobulin variable domain sequence and a light chain (LC) immunoglobulin variable domain sequence that binds to an epitope of CLDN 18.2 and does not bind to CLDN 18.1, wherein:
the HC comprises a CDR1 comprising an amino acid sequence of GYSFTGYN (SEQ ID NO: 28), a CDR2 comprising an amino acid sequence of INPYNGGT (SEQ ID NO: 29) and a CDR3 comprising an amino acid sequence of ARMGLGNAMDY (SEQ ID NO: 30) and
the LC comprises a CDR1 comprising an amino acid sequence of QNLLNSGNQKNY (SEQ ID NO: 25), a CDR2 comprising an amino acid sequence of WAS (SEQ ID NO: 26) and a CDR3 comprising an amino acid sequence of QNDYIYPLP (SEQ ID NO: 27).

21. The antibody or fragment thereof of claim 20, wherein
the HC variable region comprises EVQLQQSGPELVKPGASMKISCKASGYSFTGYNMNWVKQTHGKNLEWIGLINPYN GGTRYNQKFKGKATLTVDKSSSTAYMELLSLTSEDSAVYFCARMGLGNAMDYWG QGTSVTVSS (SEQ ID NO: 54) or an equivalent thereof; and
wherein the LC variable region comprises DIVMTQSPSSLTVTAGEKVTMSC KSSQNLLNSGNQKNYLTWYQQKPGQPPKLLIYWASTMESGVPDRFTGSGSGTDFT LTISSVQAEDLAVYYC QNDYIYPLP FGAGTKLELK (SEQ ID NO: 56), or an equivalent thereof.


SEQUENCE LISTING (107 sequences)

SEQ ID NO: 1 - protein, 12 aa, synthetic construct
  REGION 1..12: note = Description of Artificial Sequence: Synthetic peptide
QNLLNSSNQKNY

SEQ ID NO: 2 - skipped

SEQ ID NO: 3 - protein, 9 aa, synthetic construct
  REGION 1..9: note = Description of Artificial Sequence: Synthetic peptide
QQHYSTPLT

SEQ ID NO: 4 - protein, 8 aa, synthetic construct
  REGION 1..8: note = Description of Artificial Sequence: Synthetic peptide
GFTFSSYA

SEQ ID NO: 5 - protein, 7 aa, synthetic construct
  REGION 1..7: note = Description of Artificial Sequence: Synthetic peptide
ISGGGST

SEQ ID NO: 6 - protein, 14 aa, synthetic construct
  REGION 1..14: note = Description of Artificial Sequence: Synthetic peptide
GGNIYDGYPYYFDY

SEQ ID NO: 7 - protein, 10 aa, synthetic construct
  REGION 1..10: note = Description of Artificial Sequence: Synthetic peptide
ESVDNYGFSF

SEQ ID NO: 8 - skipped

SEQ ID NO: 9 - protein, 9 aa, synthetic construct
  REGION 1..9: note = Description of Artificial Sequence: Synthetic peptide
QQSNQGPLT

SEQ ID NO: 10 - protein, 8 aa, synthetic construct
  REGION 1..8: note = Description of Artificial Sequence: Synthetic peptide
GFTFSDYY

SEQ ID NO: 11 - protein, 8 aa, synthetic construct
  REGION 1..8: note = Description of Artificial Sequence: Synthetic peptide
ISDGGGDT

SEQ ID NO: 12 - protein, 9 aa, synthetic construct
  REGION 1..9: note = Description of Artificial Sequence: Synthetic peptide
ARHRGSLDY

SEQ ID NO: 13 - protein, 11 aa, synthetic construct
  REGION 1..11: note = Description of Artificial Sequence: Synthetic peptide
QSLVHSNGNTY

SEQ ID NO: 14 - skipped

SEQ ID NO: 15 - protein, 9 aa, synthetic construct
  REGION 1..9: note = Description of Artificial Sequence: Synthetic peptide
SQNTHVPRT

SEQ ID NO: 16 - protein, 8 aa, synthetic construct
  REGION 1..8: note = Description of Artificial Sequence: Synthetic peptide
GFNIKDYF

SEQ ID NO: 17 - protein, 8 aa, synthetic construct
  REGION 1..8: note = Description of Artificial Sequence: Synthetic peptide
IDPENGDT

SEQ ID NO: 18 - protein, 17 aa, synthetic construct
  REGION 1..17: note = Description of Artificial Sequence: Synthetic peptide
NVLGYGNYGHFYYAMDY

SEQ ID NO: 19 - protein, 10 aa, synthetic construct
  REGION 1..10: note = Description of Artificial Sequence: Synthetic peptide
ESVDNYGFSF

SEQ ID NO: 20 - skipped

SEQ ID NO: 21 - protein, 9 aa, synthetic construct
  REGION 1..9: note = Description of Artificial Sequence: Synthetic peptide
QQSNKVPLT

SEQ ID NO: 22 - protein, 8 aa, synthetic construct
  REGION 1..8: note = Description of Artificial Sequence: Synthetic peptide
GFTFSDYY

SEQ ID NO: 23 - protein, 8 aa, synthetic construct
  REGION 1..8: note = Description of Artificial Sequence: Synthetic peptide
ISNGGGST

SEQ ID NO: 24 - protein, 9 aa, synthetic construct
  REGION 1..9: note = Description of Artificial Sequence: Synthetic peptide
ARHRGSLDF

SEQ ID NO: 25 - protein, 12 aa, synthetic construct
  REGION 1..12: note = Description of Artificial Sequence: Synthetic peptide
QNLLNSGNQKNY

SEQ ID NO: 26 - skipped

SEQ ID NO: 27 - protein, 9 aa, synthetic construct
  REGION 1..9: note = Description of Artificial Sequence: Synthetic peptide
QNDYIYPLP

SEQ ID NO: 28 - protein, 8 aa, synthetic construct
  REGION 1..8: note = Description of Artificial Sequence: Synthetic peptide
GYSFTGYN

SEQ ID NO: 29 - protein, 8 aa, synthetic construct
  REGION 1..8: note = Description of Artificial Sequence: Synthetic peptide
INPYNGGT

SEQ ID NO: 30 - protein, 11 aa, synthetic construct
  REGION 1..11: note = Description of Artificial Sequence: Synthetic peptide
ARMGLGNAMDY

SEQ ID NO: 31 - protein, 10 aa, synthetic construct
  REGION 1..10: note = Description of Artificial Sequence: Synthetic peptide
ESVDNYGFSF

SEQ ID NO: 32 - skipped

SEQ ID NO: 33 - protein, 9 aa, synthetic construct
  REGION 1..9: note = Description of Artificial Sequence: Synthetic peptide
QQSNQGPLT

SEQ ID NO: 34 - protein, 8 aa, synthetic construct
  REGION 1..8: note = Description of Artificial Sequence: Synthetic peptide
GFSLTSYG

SEQ ID NO: 35 - protein, 7 aa, synthetic construct
  REGION 1..7: note = Description of Artificial Sequence: Synthetic peptide
IWSDGRT

SEQ ID NO: 36 - protein, 13 aa, synthetic construct
  REGION 1..13: note = Description of Artificial Sequence: Synthetic peptide
ARHGRYDPYAMDY

SEQ ID NO: 37 - DNA, 366 bp, synthetic construct
  misc_feature 1..366: note = Description of Artificial Sequence: Synthetic polynucleotide
  source 1..366: mol_type = other DNA
gaagtgaagctggtggagtctgggggaggcttagtgaagcctggagggtccctgaaactctcctgtgcagcctctggattcactttcagtagctatgccatgtcttgggttcgccagactccagagaagaggctggagtgggtcgcatccattagtggtggtggtagcacctactatccagacagtgtgaagggccgattcaccatctccagagataatgccaggaacatcctgtacctgcaaatgagctatctgaggtctgaggacacggccgtgtattactgtgcaagaggagggaatatctatgatggttacccgtactactttgactactggggccaaggcaccactctcacagtctcctca

SEQ ID NO: 38 - protein, 122 aa, synthetic construct
  REGION 1..122: note = Description of Artificial Sequence: Synthetic polypeptide
EVKLVESGGGLVKPGGSLKLSCAASGFTFSSYAMSWVRQTPEKRLEWVASISGGGSTYYPDSVKGRFTISRDNARNILYLQMSYLRSEDTAVYYCARGGNIYDGYPYYFDYWGQGTTLTVSS

SEQ ID NO: 39 - DNA, 339 bp, synthetic construct
  misc_feature 1..339: note = Description of Artificial Sequence: Synthetic polynucleotide
  source 1..339: mol_type = other DNA
gacattgtgatgacccagtctccatcctccctggctatgtcagtaggacagaaggtcactatgagctgcaagtccagtcagaaccttttaaatagtagcaatcaaaagaactatttggcctggtaccagcagaaaccaggacagtctcctaaacttctggtatactttgcatccactagggaatctggggtccctgatcgcttcacaggcagtggatctgggacagatttcactcttaccatcagcagtgtgcaggctgaagacctggcagtttattactgtcagcaacattatagcactccgctcacgttcggtgctgggaccaagctggagctgaaa

SEQ ID NO: 40 - protein, 113 aa, synthetic construct
  REGION 1..113: note = Description of Artificial Sequence: Synthetic polypeptide
DIVMTQSPSSLAMSVGQKVTMSCKSSQNLLNSSNQKNYLAWYQQKPGQSPKLLVYFASTRESGVPDRFTGSGSGTDFTLTISSVQAEDLAVYYCQQHYSTPLTFGAGTKLELK

SEQ ID NO: 41 - DNA, 348 bp, synthetic construct
  misc_feature 1..348: note = Description of Artificial Sequence: Synthetic polynucleotide
  source 1..348: mol_type = other DNA
gaagtgaagctggtggagtctgggggaggcttagtgcagcctggagggtccctgaaactctcctgtgcaacctctggattcactttcagtgactattacatgttttggattcgccagactccagagaagaggctggagtgggtcgcctccattagtgatggtggtggtgacacctattatccagacactgtaaagggccgattcaccatctccagagacaatgccaagaacaccctgtacctgcaaatgagccgtctgaagtctgaggacacagccatgtattactgtgcaagacataggggctctcttgactactggggccaaggcaccactctcacagtctcctca

SEQ ID NO: 42 - protein, 116 aa, synthetic construct
  REGION 1..116: note = Description of Artificial Sequence: Synthetic polypeptide
EVKLVESGGGLVQPGGSLKLSCATSGFTFSDYYMFWIRQTPEKRLEWVASISDGGGDTYYPDTVKGRFTISRDNAKNTLYLQMSRLKSEDTAMYYCARHRGSLDYWGQGTTLTVSS

SEQ ID NO: 43 - DNA, 333 bp, synthetic construct
  misc_feature 1..333: note = Description of Artificial Sequence: Synthetic polynucleotide
  source 1..333: mol_type = other DNA
gacattgtgctgacccaatctccagcttctttggctgtgtctctagggcagagggccaccatctcctgcagagccagcgagagtgtggataactatggctttagttttctgcactggtaccagcagaaaccaggacagccacccaaactcctcatctatcgtgcatccaacctagcatctgggatccctgccaggttcagtggcagtgggtctaggacagacttcaccctcaccattaatcctgtggagactgatgatgttgcaacctattactgtcagcaaagtaatcagggtccactcacgttcggtgctgggaccaagctggagctgaaa

SEQ ID NO: 44 - protein, 111 aa, synthetic construct
  REGION 1..111: note = Description of Artificial Sequence: Synthetic polypeptide
DIVLTQSPASLAVSLGQRATISCRASESVDNYGFSFLHWYQQKPGQPPKLLIYRASNLASGIPARFSGSGSRTDFTLTINPVETDDVATYYCQQSNQGPLTFGAGTKLELK

SEQ ID NO: 45 - DNA, 372 bp, synthetic construct
  misc_feature 1..372: note = Description of Artificial Sequence: Synthetic polynucleotide
  source 1..372: mol_type = other DNA
gaggttcagctgcagcagtctgggacagagcttgtgaggtcaggggcctcagtcaagttgtcctgcacaacttctggcttcaacattaaagactactttttacactgggtgaagcagaggcctgaacagggcctggagtggattggatggattgatcctgagaatggtgatactaaatatgccccgaagttccaggacaaggtcaccatgactgtggacacatcctccaacacagcctgcctgcacctcagcagcctgacatctgatgacactgccgtctattactgtaatgtactaggctacggtaattacggacatttttactatgctatggactactggggtcaaggaacctcagtcaccgtctcctct

SEQ ID NO: 46 - protein, 124 aa, synthetic construct
  REGION 1..124: note = Description of Artificial Sequence: Synthetic polypeptide
EVQLQQSGTELVRSGASVKLSCTTSGFNIKDYFLHWVKQRPEQGLEWIGWIDPENGDTKYAPKFQDKVTMTVDTSSNTACLHLSSLTSDDTAVYYCNVLGYGNYGHFYYAMDYWGQGTSVTVSS

SEQ ID NO: 47 - DNA, 336 bp, synthetic construct
  misc_feature 1..336: note = Description of Artificial Sequence: Synthetic polynucleotide
  source 1..336: mol_type = other DNA
gatgttgtgatgacccaaactccactctccctgcctgtcagtcttggagatcaggcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtgaatcagggacagatttcacactcaagatcagcagagtggaggctgaggatctgggaatttatttctgctctcaaaatacacatgttcctcggacgttcggtggaggcaccaagctggaaatcaga

SEQ ID NO: 48 - protein, 112 aa, synthetic construct
  REGION 1..112: note = Description of Artificial Sequence: Synthetic polypeptide
DVVMTQTPLSLPVSLGDQASISCRSSQSLVHSNGNTYLHWYLQKPGQSPKLLIYKVSNRFSGVPDRFSGSESGTDFTLKISRVEAEDLGIYFCSQNTHVPRTFGGGTKLEIR

SEQ ID NO: 49 - DNA, 348 bp, synthetic construct
  misc_feature 1..348: note = Description of Artificial Sequence: Synthetic polynucleotide
  source 1..348: mol_type = other DNA
gaagtgaagctggtggagtctgggggaggcttagtgcagcctggagggtccctgaaactctcctgtgcaacctctggattcactttcagtgactattacatgtattgggttcgccagactccagagaagaggctggagtgggtcgcatacattagtaatggtggtggttccacctattatccagacactgtgaagggccgattcaccatctccagagacaatgccaagaacaccctgtacctgcaaatgagccgtctgaagtctgaggacacagccatgtattactgtgcaagacataggggctctcttgacttctggggccaaggcaccactctcacagtctcctca

SEQ ID NO: 50 - protein, 116 aa, synthetic construct
  REGION 1..116: note = Description of Artificial Sequence: Synthetic polypeptide
EVKLVESGGGLVQPGGSLKLSCATSGFTFSDYYMYWVRQTPEKRLEWVAYISNGGGSTYYPDTVKGRFTISRDNAKNTLYLQMSRLKSEDTAMYYCARHRGSLDFWGQGTTLTVSS

SEQ ID NO: 51 - DNA, 333 bp, synthetic construct
  misc_feature 1..333: note = Description of Artificial Sequence: Synthetic polynucleotide
  source 1..333: mol_type = other DNA
gacatagtgctgacacagtctcctgcttccttagctgtatctctggggcagagggccaccatctcctgcagagccagcgaaagtgttgataattatggctttagttttatgcactggtaccagcagaaaccaggacagccacccaaactcctcatctatcgtgcatccaacctagaatctgggatccctgccaggttcagtggcagttggtctaggacagacttcaccctcaccattaatcctgtggagactgatgatgttgcaacctattactgtcagcaaagtaataaggttccgctcacgttcggtgctgggaccaagctggagctgaaa

SEQ ID NO: 52 - protein, 111 aa, synthetic construct
  REGION 1..111: note = Description of Artificial Sequence: Synthetic polypeptide
DIVLTQSPASLAVSLGQRATISCRASESVDNYGFSFMHWYQQKPGQPPKLLIYRASNLESGIPARFSGSWSRTDFTLTINPVETDDVATYYCQQSNKVPLTFGAGTKLELK

SEQ ID NO: 53 - DNA, 354 bp, synthetic construct
  misc_feature 1..354: note = Description of Artificial Sequence: Synthetic polynucleotide
  source 1..354: mol_type = other DNA
gaggtccagctgcaacagtctggacctgagctggtgaagcctggagcttcaatgaagatatcctgcaaggcttctggttactcattcactggctacaacatgaactgggtgaagcagacccatggaaagaaccttgagtggattggacttattaatccttacaatggtggtactaggtacaaccagaagttcaagggcaaggccacattaactgtagacaagtcatccagcacagcctacatggagctcctcagtctgacatctgaggactctgcagtctatttctgtgcaaggatgggacttggaaatgctatggactactggggtcaaggaacctcagtcaccgtctcctca

SEQ ID NO: 54 - protein, 118 aa, synthetic construct
  REGION 1..118: note = Description of Artificial Sequence: Synthetic polypeptide
EVQLQQSGPELVKPGASMKISCKASGYSFTGYNMNWVKQTHGKNLEWIGLINPYNGGTRYNQKFKGKATLTVDKSSSTAYMELLSLTSEDSAVYFCARMGLGNAMDYWGQGTSVTVSS

SEQ ID NO: 55 - DNA, 339 bp, synthetic construct
  misc_feature 1..339: note = Description of Artificial Sequence: Synthetic polynucleotide
  source 1..339: mol_type = other DNA
gacattgtgatgacacagtctccatcctccctgactgtgacagcaggagagaaggtcactatgagctgcaagtccagtcagaatctgttaaacagtggaaatcaaaagaactacttgacctggtaccagcagaaaccagggcagcctcctaaactgttgatctactgggcatccactatggaatctggggtccctgatcgcttcacaggcagtggatctggaacagatttcactctcaccatcagcagtgtgcaggctgaagacctggcagtttattactgtcagaatgattatatttatccgctcccgttcggtgctgggaccaagctggagctgaaa

SEQ ID NO: 56 - protein, 113 aa, synthetic construct
  REGION 1..113: note = Description of Artificial Sequence: Synthetic polypeptide
DIVMTQSPSSLTVTAGEKVTMSCKSSQNLLNSGNQKNYLTWYQQKPGQPPKLLIYWASTMESGVPDRFTGSGSGTDFTLTISSVQAEDLAVYYCQNDYIYPLPFGAGTKLELK

SEQ ID NO: 57 - DNA, 357 bp, synthetic construct
  misc_feature 1..357: note = Description of Artificial Sequence: Synthetic polynucleotide
  source 1..357: mol_type = other DNA
caggtgcagctgaaggagtcaggacctggcctggtggcgccctcacagagcctgtccatcacatgcaccatctcagggttctcattaaccagctatggtatacactgggttcgccagcctccaggaaagggtctggagtggctggtagtgatatggagtgatggaagaacaacctataattcagctctcaaatccagactgagcatcagcaaggacaactccaagagccaagttttcttaaaaatgaacagtctccaaactgatgacacagccatgtactactgtgccagacatggtagatacgacccctatgctatggactactggggtcaaggaacctcagtcaccgtctcctca

SEQ ID NO: 58 - protein, 119 aa, synthetic construct
  REGION 1..119: note = Description of Artificial Sequence: Synthetic polypeptide
QVQLKESGPGLVAPSQSLSITCTISGFSLTSYGIHWVRQPPGKGLEWLVVIWSDGRTTYNSALKSRLSISKDNSKSQVFLKMNSLQTDDTAMYYCARHGRYDPYAMDYWGQGTSVTVSS

SEQ ID NO: 59 - DNA, 333 bp, synthetic construct; identical to SEQ ID NO: 43
  misc_feature 1..333: note = Description of Artificial Sequence: Synthetic polynucleotide
  source 1..333: mol_type = other DNA

SEQ ID NO: 60 - protein, 111 aa, synthetic construct; identical to SEQ ID NO: 44
  REGION 1..111: note = Description of Artificial Sequence: Synthetic polypeptide

SEQ ID NO: 61 - protein, 118 aa, synthetic construct; identical to SEQ ID NO: 54
  REGION 1..118: note = Description of Artificial Sequence: Synthetic polypeptide

SEQ ID NO: 62 - protein, 118 aa, synthetic construct
  REGION 1..118: note = Description of Artificial Sequence: Synthetic polypeptide
EVQLVQSGAEVKKPGESLKISCKGSGYSFTGYNMNWVRQMPGKGLEWMGLINPYNGGTRYNQKFKGQVTISADKSISTAYLQWSSLKASDTAMYFCARMGLGNAMDYWGQGTTVTVSS

SEQ ID NO: 63 - protein, 118 aa, synthetic construct
  REGION 1..118: note = Description of Artificial Sequence: Synthetic polypeptide
EVQLVQSGAEVKKPGESLKISCKGSGYSFTGYNMNWVRQMPGKNLEWIGLINPYNGGTRYNQKFKGQVTISADKSISTAYLQWSSLKASDTAMYFCARMGLGNAMDYWGQGTTVTVSS

SEQ ID NO: 64 - protein, 118 aa, synthetic construct
  REGION 1..118: note = Description of Artificial Sequence: Synthetic polypeptide
EVQLVQSGAEVKKPGESLKISCKGSGYSFTGYNMNWVRQMPGKNLEWIGLINPYNGGTRYNQKFKGKATLSVDKSISTAYLQWSSLKASDTAMYFCARMGLGNAMDYWGQGTTVTVSS

SEQ ID NO: 65 - protein, 113 aa, synthetic construct; identical to SEQ ID NO: 56
  REGION 1..113: note = Description of Artificial Sequence: Synthetic polypeptide

SEQ ID NO: 66 - protein, 113 aa, synthetic construct
  REGION 1..113: note = Description of Artificial Sequence: Synthetic polypeptide
DIVMTQSPDSLAVSLGERATINCKSSQNLLNSGNQKNYLTWYQQKPGQPPKLLIYWASTMESGVPDRFSGSGSGTDFTLTISSLQAEDVAVYYCQNDYIYPLPFGQGTKLEIK

SEQ ID NO: 67 - protein, 113 aa, synthetic construct
  REGION 1..113: note = Description of Artificial Sequence: Synthetic polypeptide
DIVMTQSPDSLAVSLGERATINCKSSQNLLQSGNQKNYLTWYQQKPGQPPKLLIYWASTRESGVPDRFSGSGSGTDFTLTISSLQAEDVAVYYCQNDYIYPLPFGQGTKLEIK

SEQ ID NO: 68 - DNA, 354 bp, synthetic construct; identical to SEQ ID NO: 53
  misc_feature 1..354: note = Description of Artificial Sequence: Synthetic polynucleotide
  source 1..354: mol_type = other DNA

SEQ ID NO: 69 - DNA, 354 bp, synthetic construct
  misc_feature 1..354: note = Description of Artificial Sequence: Synthetic polynucleotide
  source 1..354: mol_type = other DNA
gaagtccagctcgtgcagtctggtgcggaggtcaaaaaacccggcgagtctctcaaaattagttgcaaaggctccggttattcattcacgggttacaatatgaactgggttcgacaaatgccgggtaagggacttgaatggatgggattgatcaacccctataacggaggtaccagatacaaccaaaaatttaagggccaggtgactattagtgcagataaaagtatctccaccgcttacctccaatggtctagtttgaaggcgtctgacactgctatgtacttttgcgcgagaatggggttgggaaatgcgatggactactggggtcagggcactacggttacggtctcctct

SEQ ID NO: 70 - DNA, 354 bp, synthetic construct
  misc_feature 1..354: note = Description of Artificial Sequence: Synthetic polynucleotide
  source 1..354: mol_type = other DNA
gaggtacagctcgtgcaatctggggccgaagtaaagaaacctggagaaagtcttaaaataagctgcaagggcagtgggtatagcttcacgggctacaatatgaactgggttagacaaatgccaggtaagaacctcgaatggataggattgatcaacccatacaatggtggtacgcgctacaaccagaaattcaaaggtcaggttaccatctctgcggataaaagcatctcaacggcgtatcttcaatggtcctcactgaaagcatccgacacagcaatgtatttttgcgctagaatgggattgggtaatgccatggactactggggccaaggcaccacggttacagtctcttcc

SEQ ID NO: 71 - DNA, 354 bp, synthetic construct
  misc_feature 1..354: note = Description of Artificial Sequence: Synthetic polynucleotide
  source 1..354: mol_type = other DNA
gaagttcagctggtgcaaagtggggctgaggtaaagaagcctggcgagagcctcaagatttcttgcaagggtagcggatacagttttacgggatataacatgaattgggttcgccagatgccggggaagaaccttgaatggataggactgataaacccctataatgggggaacccgatataatcagaagtttaagggaaaagcaactttgtcagttgacaagtctatcagcacggcctatcttcagtggtccagtctgaaagcaagcgacacggctatgtacttttgtgcacgcatggggcttggtaacgcaatggactattggggacaaggaactaccgtcactgtctcttca

SEQ ID NO: 72 - DNA, 339 bp, synthetic construct; identical to SEQ ID NO: 55
  misc_feature 1..339: note = Description of Artificial Sequence: Synthetic polynucleotide
  source 1..339: mol_type = other DNA

SEQ ID NO: 73 - DNA, 339 bp, synthetic construct
  misc_feature 1..339: note = Description of Artificial Sequence: Synthetic polynucleotide
  source 1..339: mol_type = other DNA
gacatcgtgatgacccagtccccggacagtctggcagtcagtcttggggaaagagctaccataaactgcaaatccagccaaaaccttcttaatagcggcaaccaaaagaattacttgacttggtatcagcaaaaaccgggtcagccgcccaaactcttgatatactgggcgtctacgatggaaagcggcgtccccgaccgcttcagcgggagtgggtcagggactgatttcactttgacaatcagttcccttcaggcagaggacgtagcagtctactactgtcagaatgattatatatatcctcttccgttcggccaggggacgaagttggagatcaaa

SEQ ID NO: 74 - DNA, 339 bp, synthetic construct
  misc_feature 1..339: note = Description of Artificial Sequence: Synthetic polynucleotide
  source 1..339: mol_type = other DNA
gacatcgtgatgactcaaagtcctgactcccttgctgtttcacttggcgaaagggccactatcaactgtaagagttctcagaatctcttgcaatcaggaaaccagaagaattacttgacctggtatcaacagaagcctggacaaccacctaagctcttgatttactgggctagtacaagggagtccggcgtcccagacagattttccggttctggatcaggcacggacttcactctgacaatctctagtcttcaagccgaggatgtggccgtttattattgccagaacgattacatttaccctttgccatttggtcaaggtactaagttggagataaaa

SEQ ID NO: 75 - DNA, 48 bp, synthetic construct
  misc_feature 1..48: note = Description of Artificial Sequence: Synthetic oligonucleotide
  source 1..48: mol_type = other DNA
ctcgagcccaaatcttgtgacaaaactcacacatgcccaccgtgcccg

SEQ ID NO: 76 - DNA, 81 bp, synthetic construct
  misc_feature 1..81: note = Description of Artificial Sequence: Synthetic oligonucleotide
  source 1..81: mol_type = other DNA
ttttgggtgctggtggtggttggtggagtcctggcttgctatagcttgctagtaacagtggcctttattattttctgggtg

SEQ ID NO: 77 - DNA, 126 bp, synthetic construct
  misc_feature 1..126: note = Description of Artificial Sequence: Synthetic polynucleotide
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 78 - DNA, 123 bp, synthetic construct
  misc_feature 1..123: note = Description of Artificial Sequence: Synthetic polynucleotide
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 79 - DNA, 339 bp, synthetic construct
  misc_feature 1..339: note = Description of Artificial Sequence: Synthetic polynucleotide
  source 1..339: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 80 - protein, 51 aa, Homo sapiens
PAKPTTTPAPRPPTPAPTIASQPLSLRPEACRPAAGGAVHTRGLDFACDIY

SEQ ID NO: 81 - protein, 49 aa, Mus sp.
KVNSTTTKPVLRTPSPVHPTGTSQPQRPEDCRPRGSVKGTGLDFACDIY

SEQ ID NO: 82 - protein, 51 aa, Felis catus
PVKPTTTPAPRPPTQAPITTSQRVSLRPGTCQPSAGSTVEASGLDLSCDIY

SEQ ID NO: 83 - protein, 21 aa, Homo sapiens
IYIWAPLAGTCGVLLLSLVIT

SEQ ID NO: 84 - protein, 21 aa, Mus sp.
IWAPLAGICVALLLSLIITLI

SEQ ID NO: 85 - protein, 21 aa, Rattus sp.
IWAPLAGICAVLLLSLVITLI

SEQ ID NO: 86 - protein, 42 aa, synthetic construct
  REGION 1..42: note = Description of Artificial Sequence: Synthetic polypeptide
KRGRKKLLYIFKQPFMRPVQTTQEEDGCSCRFPEEEEGGCEL

SEQ ID NO: 87 - protein, 220 aa, synthetic construct
  REGION 1..220: note = Description of Artificial Sequence: Synthetic polypeptide
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 88 - DNA, 105 bp, synthetic construct
  misc_feature 1..105: note = Description of Artificial Sequence: Synthetic polynucleotide
  source 1..105: mol_type = other DNA
acaaaaaagaagtattcatccagtgtgcacgaccctaacggtgaatacatgttcatgagagcagtgaacacagccaaaaaatccagactcacagatgtgacccta

SEQ ID NO: 89 - DNA, 108 bp, synthetic construct
  misc_feature 1..108: note = Description of Artificial Sequence: Synthetic polynucleotide
  source 1..108: mol_type = other DNA
agggaccagaggctgccccccgatgcccacaagccccctgggggaggcagtttccggacccccatccaagaggagcaggccgacgcccactccaccctggccaagatc

SEQ ID NO: 90 - protein, 112 aa, synthetic construct
  REGION 1..112: note = Description of Artificial Sequence: Synthetic polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 91 - protein, 15 aa, synthetic construct
  REGION 1..15: note = Description of Artificial Sequence: Synthetic peptide
GGGGSGGGGSGGGGS

SEQ ID NO: 92 - protein, 5 aa, synthetic construct
  REGION 1..5: note = Description of Artificial Sequence: Synthetic peptide
GGGGS

SEQ ID NO: 93 - protein, 5 aa, synthetic construct
  REGION 1..5: note = Description of Artificial Sequence: Synthetic peptide
GYNMN

SEQ ID NO: 94 - protein, 17 aa, synthetic construct
  REGION 1..17: note = Description of Artificial Sequence: Synthetic peptide
LINPYNGGTRYNQKFKG

SEQ ID NO: 95 - protein, 16 aa, synthetic construct
  REGION 1..16: note = Description of Artificial Sequence: Synthetic peptide
ARGGNIYDGYPYYFDY

SEQ ID NO: 96 - protein, 9 aa, synthetic construct
  REGION 1..9: note = Description of Artificial Sequence: Synthetic peptide
MGLGNAMDY

SEQ ID NO: 97 - protein, 17 aa, synthetic construct
  REGION 1..17: note = Description of Artificial Sequence: Synthetic peptide
KSSQNLLNSGNQKNYLT

SEQ ID NO: 98 - protein, 7 aa, synthetic construct
  REGION 1..7: note = Description of Artificial Sequence: Synthetic peptide
WASTMES

SEQ ID NO: 99 - protein, 384 aa, Homo sapiens
APTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDHGPMK

SEQ ID NO: 100 - protein, 330 aa, Homo sapiens [sequence omitted]

SEQ ID NO: 101 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 102 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 103 - protein, 452 aa, Homo sapiens
GSASAPTLFPLVSCENSPSDTSSVAVGCLAQDFLPDSITLSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVAHEALPNRVTERTVDKSTGKPTLYNVSLVMSDTAGTCY

SEQ ID NO: 104 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 105 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 106 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRMAGKPTHVNVSVVMAEVDGTCY

SEQ ID NO: 107 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC